(19) United States
(12) Patent Application Publication
(10) Pub. No.: US 2016/0145697 A1
(43) Pub. Date: May 26, 2016

(54) A Method for Determining the Vase Life or Storage History of One or More Cut Flowers, Wherein the Method Comprises Assaying Xylose Concentration or Beta-Xylosidase Expression/Activity

(71) Applicant: STICHTING DIENST LANDBOUWKUNDIG ONDERZOEK, Wageningen (NL)

(72) Inventors: Ernst Johannes Woltering, Arnhern (NL); Uilke Van Meeteren, Wageningen (NL)

(21) Appl. No.: 14/900,682

(22) PCT Filed: Jul. 2, 2014
(86) PCT No.: PCT/EP2014/064104

§ 371 Date: Dec. 22, 2015

(30) Foreign Application Priority Data
Jul. 4, 2013 (GB) 1312045.6

(51) Int. Cl. C12Q 1/68; G01N 33/00

(52) U.S. Cl. CPC C12Q 1/6895; G01N 33/0098; C12Q 2600/158; C12Q 2600/13

(57) ABSTRACT

A method for determining the vase life or storage history of one or more cut flowers, wherein the method comprises assaying a test sample obtained from the one or more cut flowers for one or more of: (a) an indicator representative of xylose concentration; (b) an indicator representative of β-xylosidase expression; and (c) an indicator representative of β-xylosidase activity; to determine a value for (each of) the one or more indicators in the test sample.

### FIELD OF THE INVENTION

The invention relates to methods for determining the storage history, and/or vase-life of cut flowers, such as roses, as well as products for use in such methods.

### BACKGROUND TO THE INVENTION

Roses and other cut flowers are being transported over longer distances (e.g. from South America to Europe or Japan) as cultivation increasingly takes place far away from major consumption areas.

A major part of product flow is currently by air. However, due to the high costs of air transport, the negative effects for the environment and the occasional lack of sufficient air freight capacity, an increasing volume of flowers is currently being shipped overseas in refrigerated (“Reefer”) containers.

At arrival, flowers are usually unpacked and re-hydrated, and thereafter brought as “fresh flowers” to e.g. a flower auction or distribution centre. At the auction, it is difficult to accurately determine how long, and under what conditions, the flowers have been held during distribution. Consequently, it is difficult to predict the “quality” and the remaining vase life of the flower product. It is, for example, difficult to judge if flowers can be stored or transported for another period of time without the risk of devaluing the flower quality versus price value balance. In particular, when, for example, supermarkets want to offer a vase life guarantee, it is of utmost importance to have reliable information about the storage history of the product.

Markers for determining storage history in flowers have previously been described for roses. A decrease in starch concentration, and corresponding increase in reducing sugars (glucose and fructose) and sucrose in petals have been suggested as markers for previously undergone storage or transport conditions (Gorin and Berkholst, 1982; Berkholst and Gonzales, 1989). However, these markers have not been introduced into commercial practice. The markers have the disadvantage that levels of starch and these sugars in petals may be very variable and dependent on e.g. cultivar variety and picking stage.

Therefore there remains in the art a need for a reliable and commercially useful test to determine the storage history and remaining vase life of cut flowers.

### SUMMARY OF THE INVENTION

The present inventors have found that levels of xylose in petals and leaves of cut roses increase with increased storage time and temperature. Moreover the inventors have shown a correlation between xylose levels and remaining vase life. Thus the inventors have identified xylose as a marker of senescence in cut flowers such as roses.

The inventors have further found that the level of expression of the gene encoding the enzyme β-xylosidase (as determined by mRNA abundance) in petals and leaves of cut roses increases (compared to the expression level at harvest) with increased storage time and temperature. It is therefore believed that expression of the β-xylosidase gene, and/or activity of the β-xylosidase enzyme will provide a further marker of senescence in cut flowers.

Apart from sucrose, glucose and fructose (and myo-inositol) that are present in flower petals of most species, other (“rare”) sugars have been found in high amounts in petals of some species. For example, in carnation petals the main sugar was found to be the sugar alcohol pinitol (Ichimura et al. 1998); in delphinium, mannitol (Ichimura et al., 2000); in chrysanthemum, L-inositol and scyllitol (Ichimura et al., 2000); and in daylily, fructan (Bieleski, 1993). In roses small amounts of xylose and Methyl-β-D-glucopyranoside were detected in petals and an increase in concentration of both compounds was observed during the vase life (Ichimura et al., 1997; 2005; 1999b, 1999a)

The exact biosynthetic route(s) leading to the accumulation of such “rare” sugars in flower petals has not been investigated in detail. In the case of roses, it has been suggested that the “rare” sugar xylose may be synthesized from myo-inositol that is also present in low amounts in rose petals (Ichimura, 1999b). However, none of these rare sugars in roses have been suggested as a marker to determine storage history or to predict remaining vase life of cut flowers.

Accordingly, in one aspect the invention provides a method for determining the vase life or storage history of one or more cut flowers, wherein the method comprises assaying a test sample obtained from the one or more cut flowers for one or more of:

(a) an indicator representative of xylose concentration;
(b) an indicator representative of β-xylosidase expression; and
(c) an indicator representative of β-xylosidase activity;
to determine a value for (each of) the one or more indicators in the test sample.

The invention further provides:
a computer-implemented method of obtaining a model for predicting vase life and/or storage history of cut flowers, wherein the method comprises:
   a) receiving a value for one or more of:
(i) an indicator representative of xylose concentration;
(ii) an indicator representative of β-xylosidase expression; and
(iii) an indicator representative of β-xylosidase activity;
   in a control sample taken from one or more control cut flowers (control indicator data), wherein the one or more control cut flowers have a known vase life and/or storage history (control vase life or storage history data); and
   b) storing the control indicator data and the control vase life or storage history data in a data storage structure that associates the control indicator data with the control vase life and/or storage history data;
a computer implemented method of predicting vase life and/or storage history of cut flowers, the method comprising:
   a) receiving a value for one or more of:
(i) an indicator representative of xylose concentration;
(ii) an indicator representative of β-xylosidase expression; and
(iii) an indicator representative of β-xylosidase activity;
   in a test sample taken from one or more cut flowers (test indicator data); and
   b) comparing the test indicator data with control indicator data obtained from one or more control flowers of known vase life and/or storage history, using a data storage structure that associates the control indicator data with the control vase life and/or storage history data, wherein the control indicator data comprises a value for one or more of:
(i) an indicator representative of xylose concentration;
(ii) an indicator representative of β-xylosidase expression; and
(iii) an indicator representative of β-xylosidase activity;
in a control sample taken from the one or more control cut flowers;
   and
a computer program which, when executed on a computer, is arranged to perform a computer-implemented method of the invention.

### DESCRIPTION OF THE FIGURES

FIG. 1: Xylose concentration in extract from petals of Avalanche roses stored dry for varying periods of time at 4° C. Data are means of 6 measurements; each measurement was performed on petal tissue derived from 2 roses.

FIG. 2: Xylose accumulation in petals of Avalanche (upper panel—A), Akito (middle panel—B) and Happy Hour (lower panel—C) roses during storage at different temperatures. Data at each time point are averages of 5 roses (n=5). From each rose, 2 outer petals were sampled.

FIG. 3: Relative gene expression levels of β-xylosidase in outer petals during storage at different temperatures. Starting level=1. Upper panel (A) cultivar (cv.) Avalanche roses; lower panel (B) cv. Happy Hour roses. Data at each time point are averages of 3 roses (n=3) measured in duplicate. From each rose, 2 outer petals were sampled.

FIG. 4: Relative gene expression of β-xylosidase in rose leaves of three different rose cultivars on day 1 to 5 during storage at 12° C. Starting level=1. Data at each time point are averages of 3 roses (n=3) measured in duplicate. From each foliate leave complex the tip and 2 outer small leaflets, closest to the tip leaflet were sampled.

FIG. 5: Xylose concentration in petals of cv. Akito roses stored at 12° C., 5° C. and 0.5° C. Each data point is an average of 4 measurements; each measurement was done on an extract of sample prepared from 5 roses

FIG. 6: Xylose concentration in leaves of cv. Akito roses stored at 12° C., 5° C. and 0.5° C. Each data point is an average of 4 measurements; each measurement was done on an extract of sample prepared from 5 roses. From each foliate leave complex the tip and 2 outer small leaflets, closest to the tip leaflet were sampled.

FIG. 7: Relative gene expression of β-xylosidase in rose petals (upper panel—A) and leaves (lower panel—B) of cv. Akito stored at 12° C., 5° C. and 0.5° C. Starting level=1. Data at each time point are averages of 3 roses (n=3) measured in duplicate.

FIG. 8: Xylose concentrations in petals of cv. Red Naomi roses following storage at different temperatures. Each data point is an average of two analyses performed on a mixed sample of outer petals from 10 roses.

FIG. 9: Xylose concentrations in leaves of red Naomi roses following storage at different temperatures. Each data point is an average of two analyses performed on a mixed sample of leaves from 10 roses.

FIG. 10: Correlation between xylose concentration in petals (in mg/g DW, horizontal axis) and the vase life of Red Naomi roses (in days, vertical axis) that were stored for various periods of time at various temperatures. A—storage at 12° C. for 2-15 days. B—storage at 8° C. for 2-19 days. C—storage at 5° C. for 2-33 days. D—storage at 0.5° C. for 4-39 days. Vase life was determined in: water+HQC (diamonds); and in 1% sugar solution+HQC (squares). Linear trend lines are plotted through data points.

FIG. 11: Correlation between xylose concentration in petals (in mg/g DW) and the vase life of cv. Red Naomi roses (in days) that were stored for various periods of time at various temperatures. Roses were stored at 12° C. for 2-15 days, at 8° C. for 2-19 days, at 5° C. for 2-33 days and at 0.5° C. for 4-39 days. Vase life was determined in: water+HQC (upper panel—A) and in 1% sugar solution+HQC (middle panel—B). The lower panel (C) shows the two vase life conditions combined. Linear trend lines are plotted through data points.

FIG. 12: Relative gene expression of β-xylosidase in petals of two separate batches (represented by two bars at each sample day; left hand bars=batch 1, right hand bars=batch 2) of rose cv. Red Naomi that were stored for different durations at different temperatures. Roses were stored at up to 15, 15, 16 and 19 days at 12° C., 8° C., 5° C. and 0.5° C., respectively. mRNA abundance was measured in 2 selected mixed samples of outer petals from 10 roses. Initial level day 0=1. Each bar represents an average of two analyses performed on a mixed sample of 2 outer petals from 10 roses.

FIG. 13: Relative gene expression of β-xylosidase in leaves of two separate batches (represented by two bars at each sample day; left hand bars=batch 1, right hand bars=batch 2) of rose cv. Red Naomi that were stored for different durations at different temperatures. Roses were stored at up to 19, 19, 33 and 37 days at 12° C., 8° C., 5° C. and 0.5° C., respectively. Initial level day 0=1. Each bar represents an average of two analyses performed on a mixed sample of 2 outer leaves from 10 roses.

FIG. 14: Concentrations of different sugars in petals of rose cultivars Akito, Red Naomi, Sphinx Gold, Passion and Aqua. For each cultivar two bars are presented. Left hand bar represents the sugar level at the start of the experiment (before storage), right hand bar represents the sugar level after storage for 12 days at 8° C. (stored). A—glucose concentrations. B—fructose concentrations. C—sucrose concentrations. D—myo-inositol concentrations. E—methyl β-D-glucopyranoside concentrations. Each data point is an average of two analyses performed on a mixed sample of outer petals from 10 roses.

FIG. 15: Xylose concentrations in petals of rose cultivars Akito, Red Naomi, Sphinx Gold, Passion and Aqua. For each cultivar two bars are presented. Left hand bar represents the xylose level at the start of the experiment (before storage), right hand bar represents the xylose level after storage for 12 days at 8° C. (stored). Each data point is an average of two analyses performed on a mixed sample of outer petals from 10 roses.

FIG. 16: Correlation between xylose concentration and the vase life of rose cultivars. Akito, Red Naomi, and Passion after 0 and 12 days of storage at 8° C.

FIG. 17: Concentrations of different sugars in petals of rose cultivars Grand Prix and Avalanche. For each cultivar two bars are presented. Left hand bar represents the sugar level at the start of the experiment (before storage), right hand bar represents the sugar level after storage for 21 days at 0.5° C. (stored). A—glucose concentrations. B—fructose concentrations. C—sucrose concentrations. D—myo-inositol concentrations. E—methyl-β-D-glucopyranoside concentrations. Each data point is an average of two analyses performed on a mixed sample of outer petals from 10 roses.

FIG. 18: Concentrations of xylose in petal of rose cultivars Grand Prix and Avalanche. For each cultivar two bars are presented. Left hand bar represents the xylose level at the start of the experiment (before storage), right hand bar represents the xylose level after storage for 21 days at 0.5° C. (stored). Each data point is an average of two analyses performed on a mixed sample of outer petals from 10 roses.

FIG. 19: Concentrations of different sugars in petals of rose cultivars Esperance and Blush. For each cultivar two bars are presented. Left hand bar represents the sugar level at the start of the experiment (before storage), right hand bar represents the sugar level after a 4 days truck-ride at 9.3° C. (stored). A—glucose concentrations. B—fructose concentrations. C—sucrose concentrations. D—myo-inositol concentrations. E—methyl-β-D-glucopyranoside concentrations. Initial level is at arrival in the Netherlands. Each data point is an average of two analyses performed on a mixed sample of outer petals from 10 roses.

FIG. 20: Concentrations of xylose in petals of rose cultivars Esperance and Blush. For each cultivar two bars are presented. Left hand bar represents the xylose level at the start of the experiment (before storage), right hand bar represents the xylose level after a 4 days truck-ride at 9.3° C. (stored Initial level is at arrival in the Netherlands. Each data point is an average of two analyses performed on a mixed sample of outer petals from 10 roses.

FIG. 21: Concentrations of different sugars in petals of rose cultivars Aqua and Passion. For each cultivar three bars are presented. Left hand bar represents the sugar level at the start of the experiment (before storage), middle bar represents the sugar level following storage scenario 1 (stored 1), right hand bar represents the sugar level following storage scenario 2 (stored 2). A—glucose concentrations. B—fructose concentrations. C—sucrose concentrations. D—myo-inositol concentrations. E—methyl-β-D-glucopyranoside concentrations. Each data point is an average of two analyses performed on a mixed sample of outer petals from 10 roses.

FIG. 22: Concentrations of xylose in petals of rose cultivars Aqua and Passion. For each cultivar three bars are presented. Left hand bar represents the xylose level at the start of the experiment (before storage), middle bar represents the xylose level following storage scenario 1 (stored 1), right hand bar represents the xylose level following storage scenario 2 (stored 2). Each data point is an average of two analyses performed on a mixed sample of outer petals from 10 roses.

FIG. 23: Correlation between xylose concentration and the vase life of rose cultivars Aqua and Passion before and after a 7 days distribution simulation. Upper panel (A): “stored 1 flowers”; lower panel (B): “stored 2 flowers”.

FIG. 24: Concentrations of different metabolites measured in petals of roses at the start (left column, black) and at the end (right column, gray) of storage. A—glucose concentrations. B—fructose concentrations. C—sucrose concentrations. D—myo-inositol concentrations. E—methyl β-D-glucopyranoside concentrations. All concentrations are expressed in mg/g DW. Where no data are presented, they were not available. The figure provides a summary of the data obtained in the examples. “Start” generally refers to a time point immediately after harvest, or on arrival in the Netherlands. “End” generally refers to a time point after a period of storage or distribution (generally at reduced temperature) as described in the Examples.

FIG. 25: Concentrations of xylose measured in petals of roses at the start (left column, black) and at the end (right column, gray) of the storage. All concentrations are expressed in mg/g DW. The Figure provides a summary of the data obtained in the Examples. “Start” generally refers to a time point immediately after harvest, or on arrival in The Netherlands. “End” generally refers to a time point after a period of storage or distribution (generally at reduced temperature) as described in the Examples.

FIG. 26: Glucose/fructose ratio in petals of roses at the start (left column, black) and at the end (right column, gray) of the storage. The Figure provides a summary of the data obtained in the Examples. “Start” generally refers to a time point immediately after harvest, or on arrival in The Netherlands. “End” generally refers to a time point after a period of storage or distribution (generally at reduced temperature) as described in the Examples.

FIG. 27: Correlation between initial levels of myo-inositol (x-axis) and xylose (y-axis). The Figure provides a summary of data obtained in the Examples.

FIG. 28: Ratio between xylose and myo-inositol at the start (left column, black) of experiment and after storage (right column, gray) for different cultivars. The Figure provides a summary of the data obtained in the Examples. “Start” generally refers to a time point immediately after harvest, or on arrival in The Netherlands. “End” generally refers to a time point after a period of storage or distribution (generally at reduced temperature) as described in the Examples.

FIG. 29: Relative gene expression level of β-xylosidase in the leaves (upper panel—A) and petals (lower panel—B) of the rose cv. Akito obtained from two different experiments. Example 2 results are shown in black (left hand bars); Example 3 results are shown in gray (right hand bars). Storage days without a representable bar have not been measured or sampled within this specific experiment. Expression levels at day zero are, by definition set to 1.

### DESCRIPTION OF THE SEQUENCES

SEQ ID NO: 1 is a consensus forward PCR primer for rose β-xylosidase.

SEQ ID NO: 2 is a consensus reverse PCR primer for rose β-xylosidase.

SEQ ID NO: 3 is a forward PCR primer for rose actin.

SEQ ID NO: 4 is a reverse PCR primer for rose actin.

SEQ ID NO: 5 is a nucleotide sequence for Arabidopsis thaliana BXL1 (NCBI Reference Sequence: NM_124313.2).

SEQ ID NO: 6 is a nucleotide sequence for Arabidopsis thaliana BXL2 (NCBI Reference Sequence: NM_100144.2).

SEQ ID NO: 7 is a nucleotide sequence for Arabidopsis thaliana BXL3 (NCBI Reference Sequence: NM_121010.2).

SEQ ID NO: 8 is a nucleotide sequence for Arabidopsis thaliana BXL4 (Gen Bank: AK221967.1).

SEQ ID NO: 9 is a protein sequence for Arabidopsis thaliana BXL1 (GenBank: AED95802.1).

SEQ ID NO: 10 is a protein sequence for Arabidopsis thaliana BXL2 (GenBank: AEE27453.1).

SEQ ID NO: 11 is a protein sequence for Arabidopsis thaliana BXL3 (GenBank: AED91439.1).

SEQ ID NO: 12 is a protein sequence for Arabidopsis thaliana BXL4 (UniProtKB/Swiss-Prot: Q9FLG1.1).

### DETAILED DESCRIPTION OF THE INVENTION

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

Throughout the description and claims of this specification, the words “comprise” and “contain” and variations of the words, for example “comprising” and “comprises”, mean “including but not limited to”, and are not intended to (and do not) exclude other moieties, additives, components, integers or steps. It will however also be understood that these terms encompass the meaning of and may in some instances be interpreted as meaning “consisting of” or “consisting essentially of”.

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

All publications, patents and patent applications mentioned in this specification are herein incorporated by reference in to the specification to the same extent as if each individual publication, patent or patent application was specifically and individually indicated to be incorporated herein by reference.

This disclosure references various internet sites and sequence database entries. The contents of the referenced internet sites and sequence database entries are incorporated herein by reference as of 27 Jun. 2013.

All references to “detectable” or “detected” are as within the limits of detection of the given assay or detection method.

The inventors have identified new markers of senescence (xylose concentration, β-xylosidase gene expression, and β-xylosidase enzyme activity) which may be used to predict remaining vase life of, and to assess likely storage history of, cut flowers, in particular, cut roses.

The inventors have shown that, in a particular cultivar, concentration of xylose in cut flower tissue (e.g. petal or leaf tissue) increases (compared to the level at harvest) with increased storage time and temperature, and that there is a correlation between the xylose concentration and the remaining vase life of the flowers. Thus, by determining the xylose concentration in a suitable tissue sample from a test batch of cut flowers, and comparing this, for example, to xylose concentration in tissue sampled from control flowers of known vase life or storage history, it is possible to determine the storage history of the test flowers and to predict the remaining vase life.

The inventors have also shown that, in a particular cultivar, expression levels of the gene encoding the enzyme β-xylosidase (referred to herein as the β-xylosidase gene) in cut flower tissue (e.g. petal or leaf tissue) increase, compared to the level at harvest, with increased storage time and temperature. By determining the level of β-xylosidase gene expression or of β-xylosidase enzyme activity in a suitable tissue sample from a test batch of cut flowers and comparing this, for example, to expression or activity levels in tissue sampled from control flowers of known vase life and storage history, it is possible to determine the storage history of the test flowers and to predict the remaining vase life.

In one aspect therefore the invention provides a method for determining the vase life or storage history of one or more cut flowers, wherein the method comprises assaying a test sample obtained from the one or more cut flowers for one or more of:

(d) an indicator representative of xylose concentration;
(e) an indicator representative of β-xylosidase expression; and
(f) an indicator representative of β-xylosidase activity;
to determine a value for (each of) the one or more indicators in the test sample.

### Vase Life

Vase-life or remaining vase-life as used herein is a measure of the quality of cut flowers, and generally describes the length of time for which cut flowers will remain acceptable from a consumer point of view. The end of vase life generally refers to the stage at which the quality of the flowers is no longer acceptable to the consumer.

Assessment of flower quality (and so of remaining vase life) is generally carried out by those skilled in the art using qualitative markers. Those skilled in the art are aware of methods for determining quality and vase life.

For example, quality of cut flowers (and end of vase-life) may be determined by monitoring the occurrence and/or severity of one or more symptoms of deterioration in the flowers. Such symptoms may occur, for example, due to physiological ageing (senescence) or due to a negative water balance (transpiration exceeding water uptake). Symptoms include: loss of petal turgescence and petal wilting; occurrence of a phenomenon called “bent neck” where the stem tissue just below the flower head shows some degree of bending; failure of the flower to open.

The precise symptoms or markers of deterioration will vary between species, but may include for example: bud opening, loss of petal turgescence (wilting), petal withering, petal in-rolling, changes in petal colour, changes in petal shape, abscission of flowers or buds, changes in fresh weight, and the appearance of disorders such as “bent neck”.

Any suitable markers of the end of vase life may be used. For example, one or more of the above markers may be used.

In practice, vase life of cut flowers may also be terminated due to (severe) microbial infection, e.g. botrytis infection of the petals. In one aspect, such flowers are removed from a data set obtained using the present methods. In one aspect, for the purposes of the present disclosure, end of vase life is not determined according to such infection.

In one aspect, vase life may be considered as the time to post-harvest senescence of the cut flowers, such that the end of vase life may be considered the stage at which the cut flowers undergo senescence (Ts), e.g. petal senescence. Markers of senescence are known in the art and include, for example, failure of the flower to open, loss of turgescence of petals, bending of the stem below the flower (“bent neck” as above).

Any suitable unit of time may be used to express vase life, for example, weeks, days, hours. Vase life may be expressed as a percentage of potential vase life with reference to freshly harvested flowers.

Changes such as those above may be assessed using a suitable numerical scale to represent the extent of the change. For example, flowers may be marked on the scale according to the extent of aging, with a higher score denoting an older flower, closer to the end of vase life. The end of vase life can be assessed as the time at which the total score for the flowers exceeds a given value.

In one aspect, flower quality may be assessed sensorially by judging, for example: the turgescence of the flowers (wilting), the flower colour, the opening rate of the flowers, and the appearance of disorders such as “bent neck”.

In the present Examples, flowers were judged on a daily basis by experienced personnel for the severity of the occurrence of symptoms of deterioration such as failure of the flower to open, loss of petal turgescence and occurrence of bent neck.

Vase life may be determined for flowers in any suitable medium, for example, in a commercial flower preservative such as Chrysal Professional 3, in tap water+bactericide at a suitable concentration, (e.g. hydroxyquinolone sulphate (HQS) at, e.g. 50 ppm) or in 1% sucrose solution+bactericide at a suitable concentration (e.g. HQS at, e.g. 50 ppm). Suitable environmental conditions, for example of temperature and light, are typically used. In one example, the conditions described in the present Examples may be used (20° C. and 12 h/12 h day/night cycle of 15 micromol/m2/s illumination from white fluorescent tubes).

### Storage History

Storage history of cut flowers generally refers to the duration of storage and/or the environmental conditions under which the flowers have been kept since the time of harvest (Tₕ). The duration of storage may be described in any suitable unit, such as weeks, days or hours. Environmental conditions may include, for example, the temperature at which the flowers have been stored, and/or the humidity (e.g. air humidity), or other specific conditions (e.g. dry, in water, packed or unpacked).

Flowers may have been transported during storage. Storage history as used herein also comprises transportation history, e.g. air or land transport.

In one aspect, storage history may be described in terms of a “temperature sum”, i.e. (temperature of storage (e.g in ° C.)×storage time (e.g. in days).

### Cut Flowers

One or more cut flowers may be tested according to the present methods. The methods may for example be used to assess a batch of flowers.

As used herein, a batch of flowers generally refers to a collection of harvested cut flowers that share a substantial part, preferably all, of their history in terms of production and/or distribution. For example, a batch of cut flowers may have been grown in the same greenhouse or growing area, and/or under the same conditions, and/or harvested at the same time. Typically, flowers in a batch have been treated in the same way since harvesting.

As used herein, “test flowers” or “test batch” refers to flowers which are to be assessed for vase life or storage history according to the present methods.

The methods of the invention are applicable to any suitable cut flowers. In one aspect, the flowers comprise those which express a β-xylosidase enzyme, as described herein.

In one aspect, the methods may be applied to cut flowers from the family Amaryllidaceae, Rosaceae, Liliaceae, Asteraceae, Iridaceae, Orchidaceae, Caryophyllaceae or any other suitable family.

In the family Rosaceae, the flowers may be of the genus Rosa. Any suitable species, cultivars and hybrids in the Rosa genus may be used. The methods may be applied to any suitable cultivar. For example, the present methods may be applied to any of the rose cultivars described in the present Examples, including Akito, Avalanche, Happy Hour, Red Naomi, Sphinx Gold, Passion, Aqua, Grand Prix, Esperance, or Blush roses.

As used herein a cultivar refers to an assemblage of plants that (a) has been selected for a particular character or combination of characters, (b) is distinct, uniform and stable in those characters, and (c) when propagated by appropriate means, retains those characters (Cultivated Plant Code).

In the family Liliaceae the flowers may be of the genus Lilium. Any suitable species in the Lilium genus may be used, for example: allium species or kniphofia species. The methods may be applied to any suitable cultivar or hybrid, for example, Lilium hybrids or tulipa hybrids.

In the family Amaryllidaceae the flowers may be of the genus Alstroemeria, Narcissus, Nerine, Amaryllus. Any suitable species in the Alstroemeria genus may be used, for example: Alstroemeria pelegrina. The methods may be applied to any suitable cultivar.

In the family Asteraceae (or Compositae) the flowers may be of the genus Chrysanthemum or Gerbera. Any suitable species in the Chrysanthemum genus may be used, for example: Chrysanthemum morifolium. Any suitable species in the Gerbera genus may be used, for example: Gerbera jamesonii. The methods may be applied to any suitable cultivar.

In the family Caryophyllaceae, the flowers may be of the genus Dianthus. Any suitable species in the Dianthus genus may be used, for example, Dianthus caryophyllus.

In the family Iridaceae, flowers may include, for example, Freesia hybrids, Iris hybrids or Gladiolus hybrids.

In the family Orchidaceae, flowers may include, for example, Cymbidium hybrids, Phalaenopsis hybrids.

In general the cut flowers have been stored for a time after harvesting. Harvesting as used herein refers to the process by which the flowers are cut from the plant and gathered. The flowers may have been transported during storage, for example, overland (e.g. by truck) and/or overseas (by air or ship freight).

Flowers may have been pretreated prior to storage. For example, flowers, e.g. roses, may be pretreated with an antimicrobial agent, e.g. a bactericide, to prevent or delay microbial (e.g. fungal or bacterial) growth in solution or in the flower (e.g. in the stem). For example, roses may have been pretreated to delay or prevent microbial infection, such as Botrytis cinerea infection. In one example, flowers, e.g. roses may have been pretreated with sodium hypochlorite at a suitable concentration (e.g. 100 ppm), or another bactericidal solution (e.g. a commercial rehydration solution such as Chrysal RVB) to delay or prevent such infection. Other pretreatments include any of those in the present Examples, including rehydration in water, at a suitable temperature, e.g. 4° C., 20° C., 1° C.

Flowers may have been stored under any suitable conditions of temperature and humidity, and for any suitable length of time.

For example, flowers may have been stored dry (e.g. in carton flower boxes), or in water. Any suitable temperature may have been used, for example, 0.5° C. to 12° C., such as 1, 2, 3, 4, 5, 6, 7, 8, 9, 10 or 11° C. Flowers may have been stored for any suitable length of time at a particular temperature and humidity, for example, 1-42 days or more, such as 2, 3, 4, 5, 10, 12, 14, 16, 18, 19, 20, 21, 22, 24, 26, 28, 30, 32, 33, 34, 36, 37, 38, 40 days or more. Flowers may have undergone more than one period of storage, at different times, temperature and/or humidity. Any of the storage conditions described in the present Examples (or any combination thereof) may have been applied to the flowers.

Flowers may have been stored in any suitable packaging.

Cut flowers may undergo a post-storage treatment before flower tissue is sampled and assayed according to the present methods. For example, flowers which have been stored and/or transported in dry conditions may be recut and/or rehydrated before flower tissue is sampled. Rehydration may be carried out in any suitable solution, e.g. a commercial rehydration solution such as Chrysal RVB, or water, and at any suitable time, e.g. 2 h, and temperature, e.g. room temperature, 5° C., 8° C. Any of the post-storage treatments described in the present Examples may be used.

### Samples

Indicators may be assayed in any suitable sample obtained from the one more cut flowers to be assessed. A sample may comprise a suitable tissue sample, or an extract obtained from a tissue sample, for example, an nucleic acid extract sample, a protein extract sample, or a sugar extract sample as described herein.

Tissue samples may comprise, for example, leaf or petal tissue. In one aspect, xylose concentration is preferably assayed in a petal tissue sample or a sample obtained therefrom. In one aspect, β-xylosidase expression or activity is preferably assayed in a leaf tissue sample or a sample obtained therefrom.

In one aspect, in flowers such as cut roses, outer petal tissue may be sampled. In one aspect, in flowers such as cut roses, leaf tissue may be sampled from the first or second leaf pair under the flower head, for example, from the first complete leaf under the head. Tissue may be sampled, for example, from the tip and two outer small leaflets closest to the tip leaflet.

If appropriate, samples, e.g. sampled petal or leaf tissue, may be collected and stored under suitable conditions before being further processed, e.g. assayed for xylose or β-xylosidase expression or activity. For example, tissue samples may be frozen in liquid nitrogen and stored at −80° C. for later analysis.

In one aspect, for a test batch of cut flowers, indicator may be assayed in tissue sampled from at least 2 flowers, for example, at least 3, 5, 10 flowers or more.

Indicator may be assayed in tissue of more than one cut flower and an average value for the indicator calculated. In another example, tissue samples (or an extract from tissue samples) from at least two cut flowers may be combined to produce a mixed sample, and indicator may be assayed in the mixed sample.

Preferably tissue samples are obtained randomly from a batch of flowers. Where tissue of more than one flower in a batch is used, tissue samples are preferably taken from the flowers contemporaneously, e.g. on the same day. Preferably, sufficient tissue is sampled to provide reasonable coverage of the batch of flowers.

Preferably assay measurements are made at least in duplicate from any particular sample.

### Indicators

The present methods comprise assaying a sample for one or more indicators, selected from: indicators representative of xylose concentration; indicators representative of β-xylosidase expression; and indicators representative of β-xylosidase activity.

Such a representative indicator generally comprises any assayable property of the sample which varies with the xylose concentration, β-xylosidase expression or β-xylosidase activity, and which can therefore be used to represent the xylose concentration, β-xylosidase expression or β-xylosidase activity in the sample. Preferably the property correlates with the xylose concentration, β-xylosidase expression or β-xylosidase activity in the sample. In one aspect, a change in the indicator is associated with a change in xylose concentration, β-xylosidase expression or β-xylosidase activity. In one aspect, change in the indicator may be caused by change in xylose concentration, β-xylosidase expression or β-xylosidase activity.

Assaying an indicator may provide a direct measure of xylose concentration, β-xylosidase expression or β-xylosidase activity (or changes therein) in a sample. Thus, an indicator representative of xylose concentration may be xylose concentration in the sample. Similarly, an indicator representative of β-xylosidase expression may be the expression level of a β-xylosidase expression product (e.g. mRNA, cDNA, protein or a fragment of any thereof), while an indicator representative of β-xylosidase activity may be the activity of the β-xylosidase enzyme as described herein.

Alternatively, assaying an indicator may provide an indirect measure of xylose concentration, β-xylosidase expression or β-xylosidase activity (or changes therein) in a sample. Examples of such indicators are described herein, and include, for example, the concentration of a product of the β-xylosidase enzyme.

In the present methods, indicator is assayed in a sample to obtain a value for the indicator in the sample. A value for an indicator may comprise an absolute value (e.g. an absolute xylose concentration or β-xylosidase protein concentration). Alternatively, a value for an indicator may comprise a relative value, determined relative to the indicator value in another sample. For example, a value for an indicator in a sample obtained at a given time t=T₁ may be determined relative to the value for the indicator in a sample obtained at a different time point (e.g. t=0).

One or more indicator values obtained from a sample or samples may be referred to as indicator data. For example, indicator data may comprise one or more of: xylose concentration; β-xylosidase expression; and β-xylosidase activity for a given sample or samples, presented as absolute or relative values. If the data is obtained from a test sample or samples, this may be referred to as test indicator data. If the data is obtained from a control sample or samples, this may be referred to as control indicator data.

### Indicators Representative of Xylose Concentration

In one aspect, the present method comprises determining one or more indicators representative of xylose concentration in a sample obtained from one or more cut flowers.

Xylose is a reducing sugar, of formula C₅H₁₀O₅.

An indicator representative of xylose concentration in a sample may be xylose concentration. Thus the present methods may comprise detecting and assaying the amount of xylose in a sample.

Xylose concentration in a sample may be determined using any suitable means. Typically, the method comprises detecting and quantifying xylose in the sample. For example, the method may comprise:
extracting or purifying sugars from the sample, e.g. a tissue sample; and
analysing the extracted sugars to determine xylose concentration.

Any suitable sugar extraction method may be used. For example, sugar may be extracted by incubation with ethanol at a suitable temperature, e.g. 75° C., for a suitable time, e.g. 20 minutes.

Xylose concentration may be assayed using enzymatic reactions, for example, by means of commercially available kits, e.g. the D-Xylose Assay Kit (Megazyme International, Ireland)

A tissue sample may be pretreated before sugar extraction and analysis. For example, frozen tissue (e.g. frozen petal or leaf tissue) may be freeze-dried and powdered before sugar extraction by incubation with ethanol as above. The extracted sample may be centrifuged, the supernatant collected and dried, e.g. in a vacuum centrifuge. Dried matter may be re-dissolved, e.g. in distilled water, and, following centrifugation, the supernatant sample analysed by HPLC.

Alternatively, frozen tissue (e.g. frozen petal or leaf tissue) may be powdered in liquid nitrogen and extraction performed directly on the sample by incubation with ethanol as above.

An indicator representative of xylose concentration in a sample may alternatively be the concentration and/or activity of another substance, which typically correlates with xylose concentration. Thus the present methods may comprise detecting and determining the concentration and/or activity of another substance (e.g. metabolite) which correlates with the concentration of xylose.

An indicator representative of xylose concentration in a sample may comprise the ratio of xylose concentration to the concentration of another substance, e.g. another metabolite or sugar, in the sample. Typically the concentration of the other molecule is substantially stable (there is substantially no detectable change in the concentration) during storage of the cut flowers. Typically the initial concentration of the molecule is correlated with the initial xylose concentration. In one example, myo-inositol (C₆H₁₂O₆) may act as a suitable reference molecule, for example in some cultivars of cut roses. Myo-inositol concentration may be determined in the same way as xylose concentration. Thus, for example, in an extract used to measure xylose, myo-inositol may be measured as another peak in the HPLC chromatogram.

Xylose concentration or an indicator representative thereof may be determined absolute or may be determined relative to another value (relative indicator value, e.g. relative xylose concentration). For example, the indicator value, e.g. xylose concentration, may be determined relative to the value of the indicator, e.g. xylose concentration, at a different time point such as t=0) (e.g. at harvest (Tₕ), or immediately before storage.

Indicators Representative of β-Xylosidase Expression and/or Activity Level

### β-Xylosidase Enzymes

As used herein, a β-xylosidase enzyme comprises an enzyme which catalyses the hydrolysis of (1->4)-β-D-xylans so as to remove successive D-xylose residues from the non-reducing termini. A β-xylosidase enzyme typically is in IUBMB (International Union of Biochemistry and Molecular Biology) category EC 3.2.1.37, and may be referred to as a xylan 1,4-β-xylosidase.

Other names for the enzyme include: 4-β-D-xylan xylohydrolase (systematic name); xylobiase; β-xylosidase; exo-1,4-β-xylosidase; β-D-xylopyranosidase; exo-1,4-xylosidase; exo-1,4-β-D-xylosidase; 1,4-β-D-xylan xylohydrolase.

β-xylosidase enzymes have been identified in a number of flowering plants. Sequences of β-xylosidase enzymes, and the nucleic acid sequences encoding them may be obtained from publicly available databases using methods known to those skilled in the art.

For example, four genes encoding four β-xylosidase enzymes have been identified in Arabidopis thaliana, as described elsewhere herein (see the Sequence information for Arabidopsis thaliana β-xylosidase enzymes section herein). The four enzymes are: BXL1 (GenBank Accession No. AED95802.1 (protein; SEQ ID NO: 9) and NM124313.2 (nucleotide; SEQ ID NO: 5)), BXL2 (GenBank Accession No. AEE27453.1 (protein; SEQ ID NO: 10) and NM100144.2 (nucleotide; SEQ ID NO: 6)), BXL3 (GenBank Accession No. AED91439.1 (protein; SEQ ID NO: 11) and NM121010.2 (nucleotide; SEQ ID NO: 7)) and BXL4 (UniProt Accession No. Q9FLG1.1 (protein; SEQ ID NO: 12) and GenBank Accession No. AK221967.1 (nucleotide; SEQ ID NO: 8)).

A β-xylosidase enzyme as referred to herein may comprise any of the above amino acid sequences and/or may be encoded by any of the above nucleotide coding sequences.

A β-xylosidase enzyme as referred to herein may comprise a homologous variant of one or more of the above β-xylosidase enzymes, as described herein. In one aspect, a β-xylosidase enzyme as referred to herein comprises an amino acid sequence which is homologous to an amino acid sequence of one or more of the β-xylosidase enzymes above. In one aspect a β-xylosidase enzyme as referred to herein is encoded by a nucleotide sequence which is homologous to a nucleotide sequence which encodes one or more of the β-xylosidase enzymes above. Homologous sequence variants are described further herein.

In one aspect, e.g. in the case of cut roses, a β-xylosidase enzyme as referred to herein may be encoded by an mRNA (or corresponding cDNA) molecule that can be amplified in a suitable PCR reaction using the forward and reverse primers described in the present Examples (SEQ ID NOS 1 and 2). Suitable PCR conditions may be determined by those skilled in the art. In one aspect, PCR conditions may comprise: Tm 58° C. for 40 cycles, and/or a primer concentration of 0.4 μM. Primer efficiency may be >96% (R² is 0.999).

In one example, the following PCR conditions may be used:
a 1.5 min denaturing step at 95° C. followed by 39 cycles of amplification (10 s at 95° C. for denaturation, 10 s at 58° C. for primer annealing and 15 s at 72° C. for extension), followed by a final extension step at 72° C. for 2 minutes. Primer concentration may be 0.4 μM.

A melting curve may be acquired by measuring the melting temperature for 5 s at 55° C. until 95° C. with an increase of 1° C. per measurement.

In one example, the conditions described in the present Examples may be used.

For some flower species, the amino acid sequence of a β-xylosidase enzyme may not be known, and/or the nucleotide sequence of a β-xylosidase gene or mRNA or cDNA may not be known. Nucleic acid primers suitable for detection and amplification of β-xylosidase gene, mRNA or cDNA in these flowers may be obtained using methods such as those described herein with respect to roses. For example, a database containing sequence ESTs (expressed sequence tags) from the test flower species may be screened with a known β-xylosidase sequence from another species, e.g. from A. Thaliana, using a suitable screening tool (e.g. BLAST). ESTs selected as homologous to the known sequence may be used for contig development, by aligning the ESTs to the known sequence used in the BLAST search. The contig may then be used to design primers, based either on β-xylosidase sequence unique to the flower species, or on β-xylosidase sequence which is conserved between the test flower species and the known flower species.

### Assaying Indicators of β-Xylosidase Expression or β-Xylosidase Activity

The present method may comprise determining one or more indicators representative of β-xylosidase expression or β-xylosidase activity in a sample obtained from one or more cut flowers, for example from petals or foliage leaves on the flower stem.

As used herein the term “expression” refers to the process whereby a protein is produced from the coding information in a gene sequence. Expression thus includes at least the following stages: transcription of a gene sequence to produce a mRNA molecule; translation of the mRNA molecule to produce a protein; any post-translational modifications that may occur to produce a protein.

An indicator of β-xylosidase expression may be the level of expression of a product of any of the stages of β-xylosidase expression, or a fragment thereof.

The present methods may thus comprise assaying a suitable sample for the product of any of the stages of β-xylosidase expression, or a fragment thereof. β-xylosidase expression may be assayed in any suitable way. For example, determining expression may comprise assaying a sample for β-xylosidase mRNA or cDNA or a fragment thereof, or assaying a sample for β-xylosidase protein (or a fragment thereof). As used herein, β-xylosidase mRNA or β-xylosidase cDNA generally refers to an mRNA or cDNA molecule which encodes a β-xylosidase enzyme.

An indicator of β-xylosidase enzyme activity may be the enzyme activity itself. β-xylosidase enzyme activity may refer to any suitable activity of the enzyme, including activity described herein. In general, the activity comprises β-D-xylosidase activity, in particular hydrolysis of (1->4)-β-D-xylans so as to remove successive D-xylose residues from the non-reducing termini.

A sample may be assayed for β-xylosidase enzyme activity using methods known in the art, and/or referred to herein.

In another aspect, an indicator of β-xylosidase enzyme expression or activity may be the concentration and/or activity of another substance, which typically correlates with the β-xylosidase enzyme expression or activity. Thus the present methods may comprise assaying a suitable sample for another molecule, the concentration or activity of which correlates with β-xylosidase expression or activity levels, and which can be used to represent β-xylosidase expression or activity.

For example, an indicator of β-xylosidase activity may comprise the concentration of a substrate or product of the enzyme. Thus, an indicator may comprise concentration of a metabolite which is produced as a result of β-xylosidase activity, e.g. xylose. Xylose concentration may be determined by any of the methods described herein.

In another example, an indicator of β-xylosidase expression may comprise the level of expression of a gene which is co-expressed with β-xylosidase. Thus the present methods may comprise assaying a sample for the product of any of the stages of expression of such a gene, or a fragment thereof.

A value for an indicator representative of β-xylosidase expression or activity, e.g. β-xylosidase expression or activity, may be determined as an absolute value or may be determined relative to another value. For example, a value for an indicator in a sample obtained at a given time point t=T₁ may be determined relative to the indicator value in a sample obtained at a different time point such as t=0 (e.g. at harvest (Tₕ), or immediately before storage.

For example, β-xylosidase expression or activity may be determined relative to the β-xylosidase expression level or activity level in another sample. Such a sample may be a sample taken at a particular time, for example time 0 (T₀) (e.g. at harvest (Tₕ), or immediately before storage).

Preferably indicator values which are expression levels are normalised (e.g. for batch to batch cDNA input and cDNA synthesis efficiency) using expression levels of genes whose expression is substantially constant in the sample, (reference genes). Reference genes include, for example, actins, GAPDH and 18S or 28S rRNA.

In one aspect the method may comprise determining mRNA or cDNA (e.g. β-xylosidase mRNA or cDNA), or a fragment of either thereof, in a suitable sample. Methods for assaying mRNA (or corresponding cDNA) levels are known in the art. Typically, nucleic acid is extracted from a sample, e.g. a tissue sample, and total RNA or total mRNA separated or purified. Methods for extracting and purifying nucleic acids such as mRNA from plant tissue are known in the art. For example, total RNA may be extracted from a ground or homogenised tissue sample using the method described in Chang et al “A simple and efficient method for isolating RNA from Pine trees” Plant Molecular Biology Reporter, Volume 11(2), 1993, 113-116.

Extracted RNA may be treated with Dnase I and column purification, as described in the present Examples. Purified RNA may be quantified by, for example, agarose gel electrophoresis and NanoDrop technology. RNA may be reverse transcribed to cDNA using known methods, e.g. iScript (Biorad) as described in the Examples.

The mRNA (or corresponding cDNA) transcription product of a given gene can be detected and quantified using methods generally known in the art, including for example, quantitative PCR methods, such as quantitative real time PCR (qRT-PCR), and nucleic acid hybridization-based methods.

Methods for carrying out quantitative PCR (qPCR) are known in the art qPCR allows quantification of the PCR reaction product. The method may include use of labelled primers and/or oligonucleotides or in the case of Taqman technology of labelled probes. Specific reaction conditions may be determined using known methods. In one embodiment, the qRT-PCR conditions described in the Examples may be used.

qRT-PCR can be used to determine a change in expression of an mRNA (or corresponding cDNA). The fold change can be calculated by determining the ratio of a test mRNA in one sample compared to another. Mathematical methods such as the Livak 2(−Delta Delta C(T)) method (2̂^{-ΔΔCt}) may be used (Schmittgen T D and Livak K J. “Analyzing real-time PCR data by the comparative C(T) method.” Nat Protoc. 2008; 3(6):1101-8) or the Pfaffl method which takes into consideration that the amplification efficiency of primers used may differ from each other. Expression ratio is calculated by: [(E_{target})^{(Ct(target;calibrator)-Ct(target;test)}]/[(E_{ref})^{(Ct(ref;calibrator)-Ct(ref;test)}] (Pfaffl, M. W., 2001. “A new mathematical model for relative quantification in real-time RT-PCR.”Nucleic Acids Res., 29:2002-2007.)

Other techniques may also be used to quantify mRNA in a sample, including, for example, transcriptome profiling by large scale RNA sequencing or Northern blot analysis using gene specific fluorescent labelled antibodies.

Preferably suitable controls are used in the present methods.

Expression of constitutively expressed genes such as reference genes may be used as positive controls, and to normalise expression levels of other test genes.

Suitable primers (forward and reverse) or probes may be designed and obtained using methods known in the art, and described herein. For example, suitable PCR primers for detection and quantification of rose β-xylosidase mRNA (or corresponding cDNA) or a fragment of either thereof (such as EST's), are described in the present Examples (SEQ ID NOS 1 & 2). Suitable PCR primers for detection and quantification of the actin “reference” mRNA (or corresponding cDNA) are described in the present Examples (SEQ ID NOS 3 & 4).

Primers or probes may be detectably labelled. Suitable labels are known in the art, and include fluorescent labels such as FITC (fluorescein), and also binding pairs such as biotin/streptavidin, wherein the biotin label may be detected after binding by a labelled streptavidin molecule.

TaqMan probes consist of a fluorophore covalently attached to the 5′-end of the oligonucleotide probe and a quencher at the 3′-end. Several different fluorophores (e.g. 6-carboxyfluorescein, acronym: FAM, or tetrachlorofluorescein, acronym: TET) and quenchers (e.g. tetramethylrhodamine, acronym: TAMRA, or dihydrocyclopyrroloindole tripeptide minor groove binder, acronym: MGB) are available (Kutyavin I V, Afonina I A, Mills A, Gorn V V, Lukhtanov E A, Belousov E S, Singer M J, Walburger D K, Lokhov S G, Gall A A, Dempcy R, Reed M W, Meyer R B, Hedgpeth J (2000), “3′-Minor groove binder-DNA probes increase sequence specificity at PCR extension temperatures”, Nucleic Acids Res., 28 (2): 655-661). The quencher molecule quenches the fluorescence emitted by the fluorophore when excited by the cycler's light source via FRET (Bustin, S A (2000). “Absolute quantification of mRNA using real-time reverse transcription polymerase chain reaction assays”. J. Mol. Endocrinol., 25 (2): 169-93.)

The present methods may comprise determining protein (e.g. β-xylosidase protein or a fragment thereof), in a sample. Any suitable means may be used to measure protein level. Methods for determining protein levels are known in the art. For example, protein levels (e.g. β-xylosidase protein levels) may be measured by HPLC using a suitable standard (e.g. β-xylosidase standard) or by substrate-enzyme assays (T. K. Ghose and V. S. Bisaria, Measurement of Hemicellulase Activities, Part 1: Xylanases, Pure & Appl. Chem., Vol. 59, No. 12, pp. 1739-1752, 1987). Protein may also be assayed, for example, using gel ELISA and specific antibodies,

The present methods may comprise determining level of β-xylosidase enzyme activity in a sample. Methods for determining activity levels are known in the art (Ghose & Bisaria 1987, vide supra). An assay for β-xylosidase enzyme activity is also described in Minic Z et al (2004) Plant Physiology, Vol 135, No. 2, 867-878, “Purification and Characterisation of Enzymes Exhibiting β-D-xylosidase Activities in Stem Tissues of Arabidopsis”. The assay described uses a reaction mixture containing 2 mM pNPX (Sigma), 0.1 M acetate buffer (pH 5.0), 2 mM sodium azide, and 50 to 100 μL of protein extract in a total volume of 0.5 mL. The reaction is carried out at 37° C. for 60 min and stopped by the addition of 0.5 mL of 0.4 M sodium bicarbonate to the assay mixture. Concentration of the resulting pNP is determined spectrophotometrically at 405 nm, and its amount estimated from a calibration curve. Specific activity is expressed as the amount of protein required to release 1 nmol/min of D-Xyl.

### Use of Test Indicator Data to Determine Vase Life or Storage History

Test indicator data may be used to provide an indication of the remaining vase life or storage history of the test flowers.

Typically this is done by comparing the test data with suitable control indicator data, obtained from control flowers whose vase life or storage history is known. Test data may be compared for example, with a single threshold value for an indicator, or may be compared with a suitable model which associates indicator data to the vase life and/or storage history of cut flowers.

Control indicator data can be derived by assaying a control sample obtained from one or more control cut flowers for one or more of:

a) an indicator representative of xylose concentration;
b) an indicator representative of β-xylosidase expression; and
c) an indicator representative of β-xylosidase activity;
to determine a value for the one or more indicators in the control sample according to the methods already described herein.

Control flowers (or control batches of flowers) may be referred to as “training flowers” or “training batches” of cut flowers. Similarly, control samples derived from such batches may be referred to as “training samples”.

In general, a control batch as used herein refers to a batch of cut flowers which is similar to the test cut flowers in terms of, for example, flower type, growth, harvesting, storage and/or distribution. For example, a control batch of flowers may be of the same genus, species or cultivar. A control batch may have been grown under the same conditions and/or have been harvested under the same conditions as the test flowers. A control batch may have been stored under the same conditions as the test flowers (insofar as the storage history of the test flowers is known). In some aspects, a control batch may have been harvested and/or stored at the same time of year as the test flowers. A control batch may have any one or more of these properties in any suitable combination.

A control or training sample refers to a sample derived from a control or training batch of cut flowers, such as any of the samples referred to herein. Typically, such a sample will be as closely as possible matched to and preferably the same as, a test sample in terms of source and/or processing e.g. a control sample may be of the same tissue type, and/or obtained in the same way.

Data obtained from a control batch or sample is generally referred to as control data (or training data).

A control batch of cut flowers (or a control sample thereof) has generally been analysed in the same way as the test batch of flowers (or test sample thereof) to determine a given indicator or indicators, e.g. xylose concentration, β-xylosidase expression and/or β-xylosidase activity. However, a control batch of cut flowers is also characterised in terms of vase life or storage history—whichever feature is being determined for the test batch of flowers.

Control batches of flowers having different vase lives and/or different storage histories can be tested according to the present methods to determine control indicator data, e.g. xylose concentration, β-xylosidase expression and/or β-xylosidase activity, and the data used to derive a model which associates indicator data, e.g. xylose concentration, β-xylosidase expression and/or β-xylosidase activity, to vase life and/or storage history. Test indicator data, e.g. xylose concentration, β-xylosidase expression and/or β-xylosidase activity, can be inputted into the model, to obtain a desired output, i.e. an indication of the storage history, e.g storage time or temperature, or predicted vase life of the test flowers.

A model might be used to obtain a relatively specific vase life for a test batch of flowers, e.g. a certain number of days or range of days, or a percentage of the potential vase life with reference to freshly harvested flowers.

Alternatively the control data may be analysed to derive categories of vase life or storage history to which test flowers can be assigned, for example “long” or “short” vase life. A model may be derived in which each category corresponds to a particular threshold indicator value, or range of values, e.g. a particular threshold value or range of values for xylose concentration, β-xylosidase expression and/or β-xylosidase activity. Thus, for example, an indicator value, e.g. xylose concentration, above threshold value “X” may indicate a vase life of “less than 5 days”, or a “short” vase life.

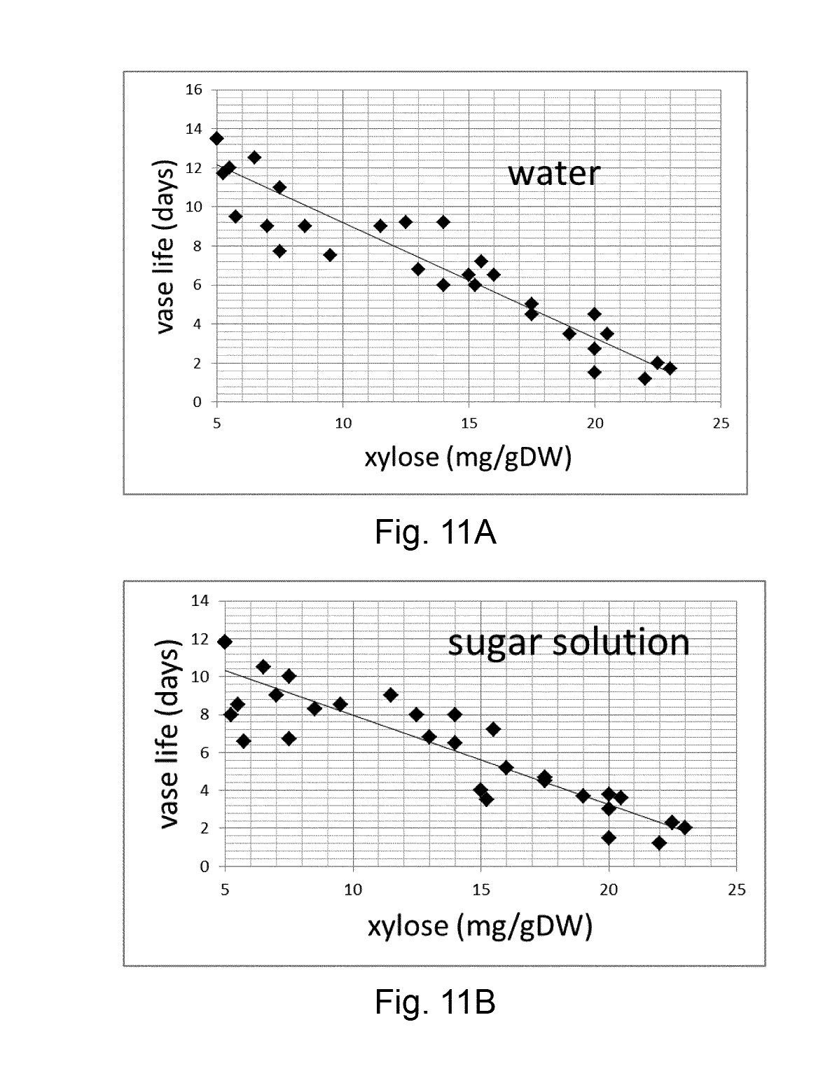

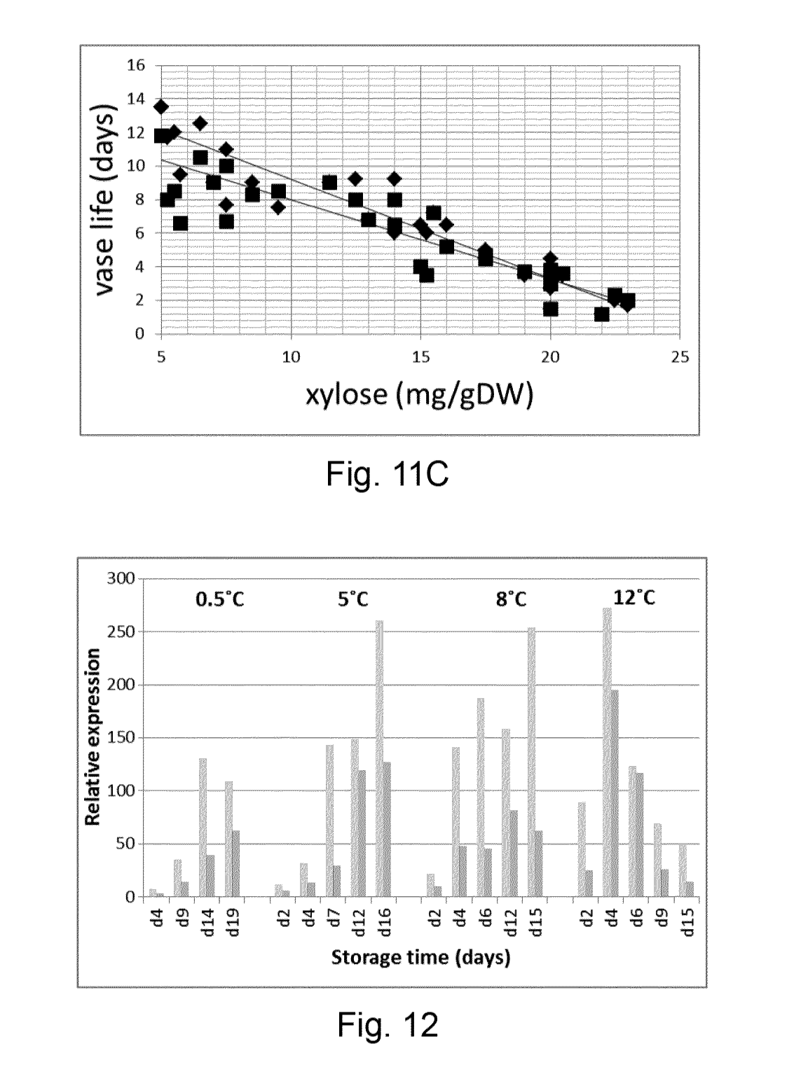

For example, FIG. 11 herein relates xylose concentration in petals with vase life of roses. Based on the data in the Figure, it can be estimated that flowers having a xylose concentration of more than 15 mg/gDW have only half the remaining vase life of fresh roses. “15 mg/gDW” may therefore be used as a threshold value for assessing vase life of test roses.

A model may, for example, be in the form of a suitable calibration curve, array, matrix, formula or algorithm. A model may comprise a computer implemented model. A model may comprise a data-storage structure as described herein.

In one aspect, the invention additionally provides a computer-implemented method of obtaining a model for predicting vase life and/or storage history of cut flowers, wherein the method comprises:

a) receiving a value for one or more of:
(i) an indicator representative of xylose concentration;
(ii) an indicator representative of β-xylosidase expression; and
(iii) an indicator representative of β-xylosidase activity;
   in a control sample taken from one or more control cut flowers (control indicator data), wherein the one or more control cut flowers have a known vase life and/or storage history (control vase life or storage history data); and
   a) b) storing the control indicator data and the control vase life or storage history data in a data storage structure that associates the control indicator data with the control vase life and/or storage history data.

The invention further provides a computer implemented method of predicting vase life and/or storage history of cut flowers, the method comprising:

(a) receiving a value for one or more of:
(i) an indicator representative of xylose concentration;
(ii) an indicator representative of β-xylosidase expression; and
(iii) an indicator representative of β-xylosidase activity;
   in a test sample taken from one or more cut flowers (test indicator data); and
   b) comparing the test indicator data with control indicator data obtained from one or more control flowers of known vase life and/or storage history, using a data storage structure that associates the control indicator data with the control vase life and/or storage history data, wherein the control indicator data comprises a value for one or more of:
(i) an indicator representative of xylose concentration;
(ii) an indicator representative of β-xylosidase expression; and
(iii) an indicator representative of β-xylosidase activity;
in a control sample taken from the one or more control cut flowers.

Receiving a value for the one or more indicators in step (a) of the method may comprise assaying a control sample for the one or more indicators, as described herein.

A data-storage structure may in one aspect be stored on a computer.

In a further aspect, the invention relates to a computer program which, when executed on a computer, is arranged to perform a computer-implemented method described herein. The computer program may be stored on a computer-readable medium.

### Primers and Probes

In one aspect the invention provides one or more nucleic acid molecules suitable for use as primers for PCR amplification of nucleic acid (e.g. cDNA or mRNA) encoding rose β-xylosidase. In one aspect the one or more nucleic acid molecules comprises a sequence of SEQ ID NO:1 or SEQ ID NO: 2 or a variant or fragment thereof. In one aspect the invention relates to a pair of PCR primers (forward and reverse) suitable for PCR amplification of nucleic acid encoding rose β-xylosidase. In one aspect the primer pair comprises a primer having the sequence of SEQ ID NO:1 or a variant or fragment thereof and a primer having the sequence of SEQ ID NO: 2 or a variant or fragment thereof.

Nucleic acid molecules for use as probes or primers typically comprise or consist of about 12-30 nucleotides, such as about 14, 16, 18, 20, 22, 24, 26, 28 nucleotides. Additionally or alternatively, in some aspects, nucleic acid molecules for use as probes or primers may have a melting temperature of between 58° C. and 62° C.

Suitable PCR conditions are described elsewhere herein.

### Kits

The invention additionally provides diagnostic kits for determining the vase life or storage history of cut flowers.

Such a kit is suitable for use in the present methods, and typically comprises one or more components for use in the methods, optionally with instructions for carrying out the methods or a part thereof.

A kit may for example, comprise unlabelled or labelled nucleic acid molecules e.g. suitable for use as primers or probes. Any one or more of the primers or probes described herein may be present, e.g. any one or more of the nucleic acid molecules having the sequences of SEQ ID NOs: 1-4, such as SEQ ID NO: 1 & 2. A kit may contain appropriate labelling and detection reagents.

Other components which may be useful for carrying out the methods described herein or a part thereof include, for example, buffers, enzymes (such as reverse transcriptase and a thermostable polymerase), nucleic acids or nucleoside triphosphates, or other reagents. A kit may comprise any one or more of such components. For example, a kit may comprise a component for use in the extraction of sugars, HPLC analysis of sugars, isolation of nucleic acids, reverse transcription of mRNA, and/or amplification reactions. A kit may comprise suitable control materials such as control nucleic acid molecules, sugar molecules or tissue samples.

A kit may comprise control data or a model or computer program for use in the present methods, as described herein.

Any one or more of the kit components may be in or on a suitable container or carrier. A kit may comprise carrying or packaging means.

A kit may contain suitable enzymes and optionally, reagents for use with the enzymes. For example, a kit may comprise one or more enzymes for use in determining xylose concentration, e.g. xylose mutarotase or xylose dehydrogenase and/or reagents such as NAD+ and ATP. In addition a kit may contain hexokinase.

### Sequence Homologs and Variants

As used herein a homolog or variant of a protein or nucleic acid sequence (e.g. a gene) refers to a protein or nucleic acid sequence that is similar in sequence and in function to the reference sequence. A species homolog refers to a similar sequence (e.g. gene and/or protein) occurring in a different species to the reference sequence.

For any nucleotide or amino acid sequence, homologous sequences may be identified by searching appropriate databases. For example, suitable databases include GenBank (available at www.ncbi.nlm.nih.gov/Genbank) and UniProt (available at http://www.ebi.ac.uk/uniprot/).

Where appropriate, databases can be searched for homologous sequences using computer programs employing various algorithms. Examples of such programs include, among others, FASTA or BLASTN for nucleotide sequences and FASTA, BLASTP, gapped BLAST, and PSI-BLAST for amino acid sequences. FASTA is described in Pearson, W R and Lipman, D J, Proc. Natl., Acad. Sci, USA, 85, 2444 2448, 1988. BLASTP, gapped BLAST, and PSI-BLAST are described in Altschul, S F, et al., Basic local alignment search tool, J. Mol. Biol., 215(3): 403 410, 1990, Altchul, S F and Gish, W, Methods in Enzymology, 266, 460 480, 1996, and Altschul, S F, et al., “Gapped BLAST and PSI-BLAST: a new generation of protein database search programs”, Nucleic Acids Res. 25:3389 3402, 1997

In addition to identifying homologous sequences, programs such as those mentioned above typically provide an indication of the degree of homology (or identity) between sequences. Determining the degree of identity or homology that exists between two or more amino acid sequences or between two or more nucleotide sequences can also be conveniently performed using any of a variety of other algorithms and computer programs known in the art. Discussion and sources of appropriate programs may be found, for example, in Baxevanis, A., and Ouellette, B. F. F., Bioinformatics: A Practical Guide to the Analysis of Genes and Proteins, Wiley, 1998; and Misener, S. and Krawetz, S. (eds.), Bioinformatics Methods and Protocols (Methods in Molecular Biology, Vol. 132), Humana Press, 1999.

Calculations of sequence homology or identity (the terms are used interchangeably herein) between sequences may be performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 75%, 80%, 82%, 84%, 85%, 86%, 87%, 88%, 89%, 90%, 91%, 92%, 93%, 94%, 95%, 96%, 97%, 98%, 99%, or 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In one embodiment, the percent identity between two amino acid sequences is determined using the Needleman et al. (1970) J. Mol. Biol. 48:444-453) algorithm which has been incorporated into the GAP program in the GCG software package (available at http://www.gcg.com), using either a BLOSUM 62 matrix or a PAM250 matrix, and a gap weight of 16, 14, 12, 10, 8, 6, or 4 and a length weight of 1, 2, 3, 4, 5, or 6. In one embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used if the practitioner is uncertain about what parameters should be applied to determine if a molecule is within a sequence identity or homology limitation of the invention) are a BLOSUM 62 scoring matrix with a gap penalty of 12, a gap extend penalty of 4, and a frameshift gap penalty of 5.

The percent identity between two amino acid or nucleotide sequences can be determined using the algorithm of Meyers et al. (1989) CABIOS 4:11-17) which has been incorporated into the ALIGN program (version 2.0), using a PAM120 weight residue table, a gap length penalty of 12 and a gap penalty of 4.

As used herein, a homologous or variant amino acid sequence generally has at least 60%, 65%, 70%, 75%, 80%, 81%. 82%, 83%, 84%, 85%, 86%, 87%, 88%, 89% 90%, 91%, 92%, 93%, 94%, 95%, 96%, 97%, 98% or 99% or more identity with the reference sequence. Thus, for example, a species homolog of the A. thaliana BXL1, BXL2, BXL3 or BXL4 protein generally has at least 60%, 65%, 70%, 75%, 80%, 81%. 82%, 83%, 84%, 85%, 86%, 87%, 88%, 89% 90%, 91%, 92%, 93%, 94%, 95%, 96%, 97%, 98% or 99% or more identity with the A. thaliana sequence. Where a homolog occurs in the same species as a reference sequence but in a different cultivar, the homolog may have any of the sequence identities listed herein.

Variants include insertions, deletions, and substitutions, either conservative or non-conservative.

In terms of amino acids, small non-polar, hydrophobic amino acids include glycine, alanine, leucine, isoleucine, valine, proline, and methionine. Large non-polar, hydrophobic amino acids include phenylalanine, tryptophan and tyrosine. The polar neutral amino acids include serine, threonine, cysteine, asparagine and glutamine. The positively charged (basic) amino acids include lysine, arginine and histidine. The negatively charged (acidic) amino acids include aspartic acid and glutamic acid. Therefore by “conservative substitutions” is intended to include combinations such as Gly, Ala; Val, Ile, Leu; Asp, Glu; Asn, Gln; Ser, Thr; Lys, Arg; and Phe, Tyr.

Due to the degeneracy of the genetic code, it is clear that any nucleic acid sequence could be varied or changed without substantially affecting the sequence of the protein encoded thereby, to provide a functional variant thereof.

As used herein, a homologous or variant nucleic acid sequence generally has at least 60%, 65% 70%, 75%, 80%, 81%. 82%, 83%, 84%, 85%, 86%, 87%, 88%, 89% 90%, 91%, 92%, 93%, 94%, 95%, 96%, 97%, 98% or 99% or more identity with the reference sequence. Thus, for example, a species homolog of the A. thaliana BXL1, BXL2, BXL3 or BXL4 nucleic acid coding sequence, or gene sequence generally has at least 60%, 65%, 70%, 75%, 80%, 81%. 82%, 83%, 84%, 85%, 86%, 87%, 88%, 89% 90%, 91%, 92%, 93%, 94%, 95%, 96%, 97%, 98% or 99% or more identity with the A. thaliana sequence. Where a homolog occurs in the same species as a reference sequence but in a different cultivar, the homolog may have any of the sequence identities listed herein

A functional variant is one in which the changes made with respect to the reference sequence do not substantially alter protein activity. For example, a functional variant of BXL1 β-xylosidase typically retains β-xylosidase protein function. In general as used herein (and unless otherwise specified), β-xylosidase homologs and variants are functional.

A fragment of a β-xylosidase nucleic acid (e.g. mRNA or cDNA) as referred to herein may be detected, or may be used for detection of a β-xylosidase mRNA or cDNA. Fragments may comprise any contiguous stretch of at least 8, 10, 12, 14, 15, 18, 20, 22, 25, 30, 40, 50, 100, 200, 500, 800, 900, 1000 or more nucleotides of a β-xylosidase nucleic acid. Such fragments may be used as PCR primers or probes for detecting β-xylosidase nucleic acid by selectively hybridizing to the β-xylosidase mRNA or cDNA.

A fragment of a β-xylosidase protein as referred to herein typically refers to a contiguous stretch of at least 8, 10, 12, 14, 15, 18, 20, 22, 25, 30, 40, 50, 100, 200, 300, 400, 500, 600 700, or more amino acids of a β-xylosidase protein.

Sequence Information for Arabidopsis thaliana β-Xylosidase Enzymes

| BXL1 nucleotide sequence (NCBI Reference Sequence: NM_124313.2) | |
|---|---|
| Gene sequence: 1-2708 | |
| Coding sequence: 84-2408 | |
| (SEQ ID NO: 5) | |
| 1 | catgaaaact aaaaaacaca aacatcacat gtatacacac atatagttac aaacacacat |
| 61 | acacaaaaca cagatatata aaaatgtctt gttataataa agcactattg atcggcaaca |
| 121 | aagtcgtcgt tatacttgta ttcctcttat gtttggttca ctcgtcagag tcacttcgac |
| 181 | cactgtttgc atgtgatcca gcaaacgggt taacccggac gctccggttc tgtcgggcca |
| 241 | atgtaccgat ccatgtgaga gttcaagatt tgctcggaag gctcacgttg caggagaaga |
| 301 | tccgcaacct cgtcaacaat gctgccgccg taccacgtct cggtattgga ggctatgagt |
| 361 | ggtggtccga ggctctccac ggcatttccg acgttggtcc aggcgctaag ttcggtggtg |
| 421 | cttttcccgg tgccaccagc ttccctcagg tcatcaccac cgcagcttct ttcaaccagt |
| 481 | ctctatggga agagatcgga cgggtggtgt ctgatgaggc aagagctatg tacaatggtg |
| 541 | gcgtggccgg tctgacatat tggagtccga atgtgaatat cttgagggac ccgcggtggg |
| 601 | gccgaggcca agaaactccc ggagaagatc ctatcgttgc cgcaaaatat gccgccagct |
| 661 | acgtccgggg acttcagggt actgctgccg gtaaccgcct taaagtcgcc gcatgttgca |
| 721 | aacattacac tgcttatgat cttgataatt ggaatggcgt cgaccgtttc cacttcaacg |
| 781 | ctaaggtcac ccaacaagat ttagaggaca catacaacgt gccattcaaa tcatgtgttt |
| 841 | acgaaggaaa agtagcgagt gtaatgtgtt cgtacaacca agtcaatgga aagcccacat |
| 901 | gtgctgatga aaatctctta aagaacacta ttcgtggtca atggcgtctc aatgggtaca |
| 961 | ttgtctcaga ttgtgactct gttgatgttt tcttcaacca acaacattac actagcactc |
| 1021 | cggaagaagc cgccgccaga tccattaaag ccggtttgga cttggactgc gggccgtttt |
| 1081 | tggcgatttt cacggaaggt gcagtgaaga aaggattgtt aacggagaat gacatcaatt |
| 1141 | tagcacttgc taatacatta acagtccaaa tgagacttgg tatgtttgat ggtaaccttg |
| 1201 | ggccgtacgc taatcttggg ccaagagatg tttgtactcc ggcccataaa catttagctc |
| 1261 | ttgaagcagc ccatcaaggg attgttcttc tcaaaaactc tgctcgctct cttccactct |
| 1321 | cccccagacg ccaccgcacc gtcgccgtga ttggtccaaa ctccgacgtc actgagacta |
| 1381 | tgatcggcaa ctatgcaggg aaagcatgcg cctatacgtc gccgttgcaa gggatttcaa |
| 1441 | gatacgcgag gacacttcac caagctggct gtgccggcgt ggcttgcaaa gggaaccaag |
| 1501 | gatttggtgc agcggaggca gcggcgcgtg aagccgacgc gacggttctt gtgatgggat |
| 1561 | tagatcagtc gatagaggca gagacacgag atcgaaccgg gcttctctta ccgggttatc |
| 1621 | aacaagacct agtgacccgt gtagctcaag cttctagagg tccagtcatt ttggtcctta |
| 1681 | tgagtggtgg accaatcgat gtaaccttcg ctaagaatga tcctcgtgtt gctgccatca |
| 1741 | tttgggctgg gtatccgggt caagcgggtg gagctgccat cgccaatatc atctttggtg |
| 1801 | ctgctaatcc cggaggaaaa ctaccaatga catggtatcc acaagattac gtggccaaag |
| 1861 | tgccaatgac ggtaatggcc atgagagcat ccggtaatta tccaggaagg acatacagat |
| 1921 | tctacaaagg tccagtagta tttccatttg ggttcggttt aagttacact accttcactc |
| 1981 | atagtttggc caaaagccca ttggcccaac tatcagtttc actctccaat ctcaactctg |
| 2041 | ccaataccat tctcaactct tcatcacact ccatcaaagt gtctcacacc aactgcaatt |
| 2101 | catttccgaa aatgcccctt cacgtcgaag tatcaaacac aggtgaattc gatggaacac |
| 2161 | acacggtgtt tgtatttgct gagccgccga taaacggaat aaaaggattg ggtgtgaaca |
| 2221 | aacaattgat agcgttcgag aaggttcatg tcatggcagg ggcaaaacag accgttcaag |
| 2281 | ttgatgttga tgcttgcaag catcttggtg tagtggatga gtatggaaag aggagaatcc |
| 2341 | caatgggtga gcataagctg cacattggtg accttaaaca tactattttg gtccaaccgc |
| 2401 | aactttgacg gacgcataaa aaaaccaaca aataaggaaa gcattttaac aaagtggagt |
| 2461 | gtttcctctt atttatataa tatagagaga taggtttatt ttcttatgaa attattactc |
| 2521 | aagaaagtat gaatttgtaa agaaccacca aagggttgct tcttggttgt gtcttttttc |
| 2581 | ctttaatttt tttttggacc aaaggtattg taacttgtgg ctcccaacat aagaaataca |
| 2641 | aggagccctt gtaaaatgct gaaactaaag ataaatgata aatttgatat atactggatt |
| 2701 | tttagagt |
| BXL2 nucleotide sequence (NCBI Reference Sequence: NM_100144.2) | |
| Gene sequence: 1-2584 | |
| Coding sequence: 89-2395 | |
| (SEQ ID NO: 6) | |
| 1 | aagtctttca tcacatttcc atttttctct cttccataaa acccaaaaga aactaggaag |
| 61 | aggagtaaat aatatttgtt ttaaagaaat gattctccac aaaatggcgt tcttggccgt |
| 121 | tattctcttc ttcttgataa gcagcagcag cgtttgcgtt catagccgtg aaacgtttgc |
| 181 | ttgcgataca aaggacgcag caacagctac actgagattc tgccagcttt cagttcctat |
| 241 | accggagaga gtcagagatt tgatcggacg gttgacattg gccgagaaag tgagcttgtt |
| 301 | agggaacact gcggcggcga taccacgtct aggaatcaaa gggtacgagt ggtggtcgga |
| 361 | ggctttacac ggcgtttcaa atgtgggacc cggtactaag ttcggtgggg tttaccctgc |
| 421 | agccaccagt ttccctcaag tcatcaccac cgttgcttct ttcaatgcct ccttgtggga |
| 481 | atccatcgga cgggttgtgt caaatgaggc cagggccatg tacaacggtg gagttggtgg |
| 541 | gcttacgtat tggagcccaa acgttaacat attgagggac ccacgttggg gacgtggaca |
| 601 | ggaaactccc ggtgaagatc cagtagtagc cggtaaatac gcagcgagct acgtcagagg |
| 661 | gttacaggga aacgaccgta gccggttaaa agtagctgct tgttgcaaac atttcacagc |
| 721 | ttacgatctc gataactgga acggcgtcga cagattccat ttcaacgcta aggtaagcaa |
| 781 | gcaagacata gaagacacgt tcgacgtacc gttccgtatg tgtgttaaag aaggtaacgt |
| 841 | tgcgagcatt atgtgttcgt acaatcaagt taatggtgtt cctacatgtg ctgatcctaa |
| 901 | tctcctcaag aagaccatac gcaatcaatg gggtctcaac gggtatatcg tgtctgattg |
| 961 | tgactctgtc ggtgttttgt acgataccca acattacact ggtactcctg aagaagctgc |
| 1021 | cgctgattcc atcaaagctg gcttggattt agattgtggg ccatttctag gagcccatac |
| 1081 | aatcgatgcg gtgaagaaaa acttgttgcg tgagtccgat gttgataatg ccttaatcaa |
| 1141 | cacgctaaca gtccaaatga gactaggaat gtttgatggc gatatagcgg ctcaaccgta |
| 1201 | cggacacctt ggaccggcac acgtgtgtac accggttcac aaaggactag ctctcgaagc |
| 1261 | agctcaacaa ggaatcgtcc tactcaaaaa tcacggctcg tctctacctc tctcaagcca |
| 1321 | acgtcaccga actgtcgccg taattggacc taattcagac gctacggtca caatgattgg |
| 1381 | taattatgca ggggttgctt gtggatatac cagtccggtt caaggtatta ccggttatgc |
| 1441 | tcgaaccatt catcaaaagg gttgcgtgga cgtacactgc atggatgata gattgttcga |
| 1501 | tgccgcggtt gaagcggctc gtggagctga tgcgacggtt cttgtgatgg gtttggatca |
| 1561 | gtctattgaa gcggagttca aggacagaaa cagtttgctt ttgcctggga aacaacaaga |
| 1621 | gcttgtctct agagttgcta aggccgctaa aggcccagtt atcttagtat tgatgtctgg |
| 1681 | tgggcctatc gatatatctt ttgctgagaa ggatcggaaa attccagcga ttgtttgggc |
| 1741 | cgggtatccg ggtcaagaag gtggtaccgc aatcgccgat atcttattcg gcagtgctaa |
| 1801 | tcccggagga aagcttccga tgacttggta tccgcaagat tatttaacca atttaccaat |
| 1861 | gacagaaatg tcgatgcggc cggtccattc gaagcggatc ccgggtcgga cttaccggtt |
| 1921 | ctacgacggt ccagttgttt acccgttcgg gcatggtttg agttacacgc gctttactca |
| 1981 | caacatagcc gacgcgccaa aagtgattcc tatagctgtt cgtggaagaa acggcaccgt |
| 2041 | ttcagggaaa tcaatccgtg tgacgcacgc taggtgtgat cgtctctctc tcggagtcca |
| 2101 | cgtggaagtt actaacgttg gctcgagaga tgggacgcac acaatgcttg tgttctcggc |
| 2161 | tccgccgggt ggagaatggg ctccgaagaa acagctggtt gcttttgaga gagtacacgt |
| 2221 | ggcggttggg gagaagaagc gtgtgcaggt gaatatacac gtgtgtaagt atttaagtgt |
| 2281 | agtggaccga gccgggaacc gaaggattcc gatcggtgat catgggattc atattggaga |
| 2341 | tgagagtcat acggtgtcgc ttcaagcttc tactcttgga gtcatcaagt cttgactctg |
| 2401 | tttttttctt ttcacttttc ttgttgttcc caaaatattt ttaagagatt ttaatgtttc |
| 2461 | taacgaaacg aatttgaaaa aggaaataca aaactagaag aaaatctgtt tcttataatt |
| 2521 | caaaagatgt atttaaaatt gaattgtatg gcctcggatt ttttaaaata aaggttgttt |
| 2581 | tcgg |
| BXL3 nucleotide sequence (NCBI Reference Sequence: NM_121010.2) | |
| Gene sequence: 1-2430 | |
| Coding sequence: 37-2358 | |
| (SEQ ID NO: 7) | |
| 1 | acaaaccaca acaaaaaatc tcgagacaaa gatacaatgg cgagccgaaa cagagcactc |
| 61 | ttctctgttt ccactctttt cctctgtttc atcgtctgca tctccgagca atcgaacaat |
| 121 | cagtcttctc cagtcttcgc ctgtgacgtc accggaaacc cttctcttgc cgggcttaga |
| 181 | ttctgcaacg cggggttgag tatcaaagcc cgagtcaccg atcttgtcgg aagattgacg |
| 241 | ttggaggaga aaatcgggtt tttgaccagc aaagctatcg gcgttagccg ccttgggatt |
| 301 | ccgtcttaca aatggtggtc ggaggcactt catggcgtct ctaacgtcgg aggtggtagt |
| 361 | cggttcaccg gtcaagtccc tggcgccact agcttcccac aagttatact cacggccgct |
| 421 | tctttcaatg tgtctttgtt ccaagccatt ggcaaggttg tatcgacaga ggcgagggca |
| 481 | atgtacaatg tgggatcagc cggtttaacg ttttggtcac ctaatgtgaa catattccgg |
| 541 | gacccgagat ggggaagagg acaagagact cccggtgagg acccaacact ctcaagcaaa |
| 601 | tacgcagtgg cctacgttaa aggtcttcag gagactgacg gtggagatcc taaccgtctc |
| 661 | aaagtcgctg cttgctgcaa acactacacc gcctatgata ttgacaattg gagaaatgtc |
| 721 | aatcgtctca ctttcaacgc tgtggtaaac caacaagatc tggctgatac gtttcaacca |
| 781 | ccgttcaaga gctgtgtggt tgatgggcat gttgctagtg tcatgtgttc ttacaaccaa |
| 841 | gttaacggta aaccaacatg tgccgatcct gatctgcttt ccggtgtgat ccgcggacaa |
| 901 | tggcagctca acggatacat tgtttcggat tgtgattcgg tagatgtgtt gttcagaaaa |
| 961 | caacactatg ctaagactcc agaagaagct gtggccaaat ctctattggc aggtttggat |
| 1021 | ttgaattgtg atcatttcaa tggtcaacac gcgatgggag cggtcaaggc gggtttggta |
| 1081 | aacgaaacag ctattgacaa agcgatttca aacaatttcg cgactctgat gcgtttaggg |
| 1141 | ttcttcgatg gagaccctaa gaagcagctc tacggtggtc ttggtcctaa ggatgtttgc |
| 1201 | accgctgata accaagaact cgcaagagat ggcgcaagac aaggcattgt cttgcttaag |
| 1261 | aactctgctg gttcgcttcc gctctcacct tccgcaatca aaacattagc cgtgatcgga |
| 1321 | ccaaacgcca atgccacaga aacaatgatc ggaaactacc acggtgtacc atgcaagtac |
| 1381 | acaacgccgc ttcagggatt ggcagaaacg gtgtcgtcta cctatcaact gggatgtaac |
| 1441 | gtggcttgcg tagatgcgga tataggctca gccgtggatc tggctgcttc tgcggatgct |
| 1501 | gttgtgctcg tggtgggcgc agaccaatca attgagaggg agggccatga ccgagtcgac |
| 1561 | ctgtatcttc ctggaaagca gcaagagctt gtgactcgag ttgctatggc agcaagagga |
| 1621 | ccggtggtgc tagttatcat gtccggtgga ggatttgaca ttacattcgc caagaatgat |
| 1681 | aaaaagatca caagcataat gtgggtcgga taccctggtg aagccggtgg tctcgccatt |
| 1741 | gctgacgtta tcttcggacg tcataatccg agtggaaatt tgccgatgac gtggtatcct |
| 1801 | caatcgtacg tggaaaaagt tccgatgtca aatatgaaca tgagacccga caaatcaaag |
| 1861 | gggtatccgg gtcggagtta caggttttac accggagaaa ccgtatacgc cttcgcagac |
| 1921 | gcgcttacct acactaaatt cgaccatcag ctaatcaaag cgccaagact cgtctctctc |
| 1981 | agtctcgacg agaaccaccc ttgccgatca tcggagtgcc aatctttgga cgcgatcgga |
| 2041 | cctcactgcg agaacgccgt tgagggagga tcggatttcg aggttcattt gaatgtaaag |
| 2101 | aacaccggag acagagcggg aagccacacg gtgtttctgt tcacgacgtc gccgcaagta |
| 2161 | cacggatctc cgattaagca actactagga tttgagaaga ttcgtctggg aaagagtgaa |
| 2221 | gaagcggtgg ttaggtttaa cgtcaatgtg tgtaaggatc tgagtgtggt tgatgagacc |
| 2281 | gggaagagga aaatcgcgtt aggtcatcat cttctccatg taggaagctt gaaacactct |
| 2341 | ttgaacatta gtgtttgatt cgacggctcg ttttgttttt aacttaagat attaattatg |
| 2401 | gtaataaaat gagatagcaa ttttaaaatc |
| BXL4 nucleotide sequence (GenBank: AK221967.1) | |
| Gene sequence (partial): 1-1656 | |
| Coding sequence(partial): 1-1572 | |
| (SEQ ID NO: 8) | |
| 1 | agttatgtgg ttgatgggaa tgtggcgagt gttatgtgtt cttacaatca agttaacggc |
| 61 | aaaccgacat gcgctgatcc agatctgctc tctggtgtta tccgcggtga atggaaatta |
| 121 | aatgggtaca ttgtttcaga ttgtgattca gtagatgtct tgtataagaa ccaacactat |
| 181 | acaaagactc cagctgaagc tgcagccata tctatattgg caggtttgga tttaaactgt |
| 241 | ggttcattct tgggtcaaca tacagaggaa gcagttaagt cgggtttggt aaacgaggca |
| 301 | gctatcgata aagcgatttc gaacaacttt ttgaccctta tgcgtttagg attctttgat |
| 361 | ggaaacccaa agaaccaaat ctatggcggg ttaggtccta ccgacgtttg cacgtctgcg |
| 421 | aatcaagagc tagcagcaga tgcagcaaga caaggcattg ttctactcaa gaatactgga |
| 481 | tgcttaccgc tttctcctaa atcgatcaaa acactagccg tgattggacc aaacgcgaat |
| 541 | gtcaccaaaa caatgattgg aaactacgaa ggcacgccgt gtaaatacac aacaccacta |
| 601 | caaggactag ccgggacggt atctacaaca tatctaccag gctgctccaa tgtagcttgt |
| 661 | gctgtagcgg atgtagccgg cgccacgaaa ctagcagcca ctgcagatgt gtctgtgctt |
| 721 | gtgatcggtg ccgatcaatc aatcgaggca gagagccgag acagagtcga cctgcgtctt |
| 781 | cctggacagc aacaagagct ggtgatccaa gtggctaaag cagcaaaagg accggtcttg |
| 841 | ctcgtcatta tgtccggtgg aggtttcgat attacattcg ctaagaatga cccaaagatc |
| 901 | gccggaattt tgtgggttgg ttatcccgga gaagccggtg gtatcgccat tgctgatatc |
| 961 | atctttggcc gttataatcc aagtgggaaa ttaccgatga cgtggtatcc acagtcgtat |
| 1021 | gtagagaaag ttccgatgac aataatgaac atgagacccg ataaagcaag cgggtatccg |
| 1081 | ggtcggactt accgattcta caccggagaa acagtatacg cattcggaga tggactcagc |
| 1141 | tacaccaaat tcagtcacac tttagtcaaa gctccaagtc tcgtttctct cggtctcgaa |
| 1201 | gagaatcacg tttgccgatc atcggaatgt caatcgctag acgcgatcgg accgcactgc |
| 1261 | gaaaacgctg tctccggcgg tggatcggcg tttgaagttc atatcaaggt acgaaacgga |
| 1321 | ggagatagag aagggattca cacggtgttt ctattcacga cgccgccggc gattcacgga |
| 1381 | tcgccgagga agcatttggt aggattcgag aagattcgat tggggaagag ggaagaagcg |
| 1441 | gtggttaggt ttaaggtaga gatatgtaaa gatctgagtg tggttgatga gattgggaag |
| 1501 | aggaagattg gtttgggaaa gcatcttctt catgtcggag atttaaaaca ttccttaagc |
| 1561 | attagaatct gattctatat ttttatttga ggaagaaaaa aagaatatta atatgcttaa |
| 1621 | gcttttgcaa gttggaaaag aaaagtaata aaaaaa |
| BXL1 protein sequence (GenBank: AED95802.1) | |
| (SEQ ID NO: 9) | |
| 1 | mscynkalli gnkvvvilvf llclvhsses lrplfacdpa ngltrtlrfc ranvpihvrv |
| 61 | qdllgrltlq ekirnlvnna aavprlgigg yewwsealhg isdvgpgakf ggafpgatsf |
| 121 | pqvittaasf nqslweeigr vvsdearamy nggvagltyw spnvnilrdp rwgrgqetpg |
| 181 | edpivaakya asyvrglqgt aagnrlkvaa cckhytaydl dnwngvdrfh fnakvtqqdl |
| 241 | edtynvpfks cvyegkvasv mcsynqvngk ptcadenllk ntirgqwrln gyivsdcdsv |
| 301 | dvffnqqhyt stpeeaaars ikagldldcg pflaiftega vkkglltend inlalantlt |
| 361 | vqmrlgmfdg nlgpyanlgp rdvctpahkh laleaahqgi vllknsarsl plsprrhrtv |
| 421 | avigpnsdvt etmignyagk acaytsplqg isryartlhq agcagvackg nqgfgaaeaa |
| 481 | areadatvlv mgldqsieae trdrtglllp gyqqdlvtrv aqasrgpvil vlmsggpidv |
| 541 | tfakndprva aiiwagypgq aggaaianii fgaanpggkl pmtwypqdyv akvpmtvmam |
| 601 | rasgnypgrt yrfykgpvvf pfgfglsytt fthslakspl aqlsvslsnl nsantilnss |
| 661 | shsikvshtn cnsfpkmplh vevsntgefd gthtvfvfae ppingikglg vnkqliafek |
| 721 | vhvmagakqt vqvdvdackh lgvvdeygkr ripmgehklh igdlkhtilv qpql |
| BXL2 protein sequence (GenBank: AEE27453.1) | |
| (SEQ ID NO: 10) | |
| 1 | milhkmafla vilfflisss svcvhsretf acdtkdaata tlrfcqlsvp ipervrdlig |
| 61 | rltlaekvsl lgntaaaipr lgikgyewws ealhgvsnvg pgtkfggvyp aatsfpqvit |
| 121 | tvasfnaslw esigrvvsne aramynggvg gltywspnvn ilrdprwgrg qetpgedpvv |
| 181 | agkyaasyvr glqgndrsrl kvaacckhft aydldnwngv drfhfnakvs kqdiedtfdv |
| 241 | pfrmcvkegn vasimcsynq vngvptcadp nllkktirnq wglngyivsd cdsvgvlydt |
| 301 | qhytgtpeea aadsikagld ldcgpflgah tidavkknll resdvdnali ntltvqmrlg |
| 361 | mfdgdiaaqp yghlgpahvc tpvhkglale aaqqgivllk nhgsslplss qrhrtvavig |
| 421 | pnsdatvtmi gnyagvacgy tspvqgitgy artihqkgcv dvhcmddrlf daaveaarga |
| 481 | datvlvmgld gsieaefkdr nslllpgkqq elvsrvakaa kgpvilvlms ggpidisfae |
| 541 | kdrkipaivw agypgqeggt aiadilfgsa npggklpmtw ypqdyltnlp mtemsmrpvh |
| 601 | skripgrtyr fydgpvvypf ghglsytrft hniadapkvi piavrgrngt vsgksirvth |
| 661 | arcdrlslgv hvevtnvgsr dgthtmlvfs appggewapk kqlvafervh vavgekkrvq |
| 721 | vnihvckyls vvdragnrri pigdhgihig deshtvslqa stlgviks |
| BXL3 protein sequence (GenBank: AED91439.1) | |
| (SEQ ID NO: 11) | |
| 1 | masrnralfs vstlflcfiv ciseqsnnqs spvfacdvtg npslaglrfc naglsikarv |
| 61 | tdlvgrltle ekigfltska igvsrlgips ykwwsealhg vsnvgggsrf tgqvpgatsf |
| 121 | pqviltaasf nvslfqaigk vvstearamy nvgsagltfw spnvnifrdp rwgrgqetpg |
| 181 | edptlsskya vayvkglqet dggdpnrlkv aacckhytay didnwrnvnr ltfnavvnqq |
| 241 | dladtfqppf kscvvdghva svmcsynqvn gkptcadpdl lsgvirgqwq lngyivsdcd |
| 301 | svdvlfrkqh yaktpeeava ksllagldln cdhfngqham gavkaglvne taidkaisnn |
| 361 | fatlmrlgff dgdpkkqlyg glgpkdvcta dnqelardga rqgivllkns agslplspsa |
| 421 | iktlavigpn anatetmign yhgvpckytt plqglaetvs styqlgcnva cvdadigsav |
| 481 | dlaasadavv lvvgadqsie reghdrvdly lpgkqqelvt rvamaargpv vlvimsgggf |
| 541 | ditfakndkk itsimwvgyp geagglaiad vifgrhnpsg nlpmtwypqs yvekvpmsnm |
| 601 | nmrpdkskgy pgrsyrfytg etvyafadal tytkfdhqli kaprlvslsl denhpcrsse |
| 661 | cqsldaigph cenaveggsd fevhlnvknt gdragshtvf lfttspqvhg spikqllgfe |
| 721 | kirlgkseea vvrfnvnvck dlsvvdetgk rkialghhll hvgslkhsln isv |
| BXL4 protein sequence (UniProtKB/Swiss-Prot: Q9FLG1.1) | |
| (SEQ ID NO: 12) | |
| 1 | mgssspltrr nrappssvss vyliflcffl yflnfsnaqs spvfacdvaa npslaaygfc |
| 61 | ntvlkieyrv adlvarltlq ekigflvska ngvtrlgipt yewwsealhg vsyigpgthf |
| 121 | ssqvpgatsf pqviltaasf nvslfqaigk vvstearamy nvglagltyw spnvnifrdp |
| 181 | rwgrgqetpg edpllaskya sgyvkglqet dggdsnrlkv aacckhytay dvdnwkgver |
| 241 | ysfnavvtqq dmddtyqppf kscvvdgnva svmcsynqvn gkptcadpdl lsgvirgewk |
| 301 | lngyivsdcd svdvlyknqh ytktpaeaaa isilagldln cgsflgqhte eavksglvne |
| 361 | aaidkaisnn fltlmrlgff dgnpknqiyg glgptdvcts anqelaadaa rqglvllknt |
| 421 | gclplspksi ktlavigpna nvtktmigny egtpckyttp lqglagtvst tylpgcsnva |
| 481 | cavadvagat klaatadvsv lvigadqsie aesrdrvdlh lpgqqqelvi qvakaakgpv |
| 541 | llvimsgggf ditfakndpk iagilwvgyp geaggiaiad iifgrynpsg klpmtwypqs |
| 601 | yvekvpmtim nmrpdkasgy pgrtyrfytg etvyafgdgl sytkfshtlv kapslvslgl |
| 661 | eenhvcrsse cqsldaigph cenavsgggs afevhikvrn ggdregihtv flfttppaih |
| 721 | gsprkhlvgf ekirlgkree avvrfkveic kdlsvvdeig krkiglgkhl lhvgdlkhsl |
| 781 | siri |

### EXAMPLES

The invention will now be described by way of specific Examples and with reference to the accompanying Figures, which are provided for illustrative purposes only and are not to be construed as limiting upon the teachings herein.

### Materials and Methods

### Plant Material and Storage

Most experiments were carried out with roses sourced from commercial growers in the Netherlands. Flowers were either dry or with their cut stem in tap water transported to Wageningen (approximately 3 h journey). In some cases, roses from Ecuador were used, after being transported by plane to The Netherlands.

At arrival, flower heads were dipped in 100 ppm sodium hypochlorite in some experiments, in order to prevent development of Botrytis cinerea. Flowers were then rehydrated overnight at 1-5° C. in water or in a commercial rehydration solution (Chrysal RVB, Chrysal International, Naarden, The Netherlands) containing a biocide and surfactant. Following rehydration and at the start of the storage, from 20-40 flowers, petals of the outer whorl and leaves from the first or second leaf pair under the flower head were collected and frozen in liquid nitrogen and stored at −80° C. until needed (time zero sample). Remaining flowers were stored dry in carton flower boxes at different temperatures for different periods of time. At regular intervals, 20-40 flowers were removed from storage, recut and rehydrated using either water or a commercial rehydration solution for approximately 2 h at 5° C. or at room temperature. Thereafter, samples were collected from petals and leaves (as described above) and frozen in liquid nitrogen and stored at −80° C. for later analysis of sugars and, in some experiments, mRNA abundance.

In some experiments, the vase life of the flowers, at time zero or stored for different periods of time, was determined.

### Vase Life Determination

Vase life was executed in a commercial flower preservative (Chrysal Professional 3—Chrysal International, Naarden, The Netherlands), in tap water+the bactericide HQS (hydroxyquinoline sulphate) at 50 ppm or in 1% sucrose solution+50 ppm HQS, depending on the experimental set up. Vase life evaluation rooms were at 20° C. and 12/12 h day night cycle of 15 micromol/m2/s illumination from white fluorescent tubes. Quality evaluation was performed sensorial by judging the turgescence of the flowers (wilting), the color, opening rate and the appearance of disorders such as bent neck and botrytis infection rate. This was done by experienced personnel using different standard scales to rate different quality artributes and using a range of photographs as an indication of acceptability levels. Together, these symptoms determined the “vase life”. Vase life was considered terminated when the flower was no longer acceptable from a consumer acceptance point of view.

If vase life was found to be terminated by severe botrytis infection these flowers were removed from the data set.

### Sugar Analysis

In most experiments, frozen petal or leaf samples were freeze-dried and powdered and sugars from 15 mg of powder were extracted using 5 ml of 80% ethanol in a shaking water bath at 75° C. for 20 min. Following centrifugation, 1 ml of the supernatant was dried in a vacuum centrifuge for 2 h. Dried matter was re-dissolved in 1 ml of distilled water and, following centrifugation, the supernatant was used for sugar analysis with HPLC.

In example 4 frozen samples were powdered in liquid nitrogen and extraction was directly performed (without freeze drying) on 250 mg of sample using 5 ml of 80% ethanol in a shaking water bath at 75° C. for 20 min.

Carbohydrates were analysed on a Dionex ICS5000 HPAEC system (Thermo Scientific, Sunnyvale Calif., USA) equipped with a CarboPac PA1 (2×250 mm) column using 45 mM NaOH as eluent.

### Starch Analysis

The residues of the carbohydrate analysis as described above were washed three times with 80% ethanol and dried in a vacuum centrifuge.

Starch was converted to glucose using 2 ml of a thermostable α-amylase solution (Serva 13452, 1 mg/ml H₂O) for 30 min at 90° C. followed by addition of 1 ml amyloglucosidase solution (Fluka 10115, 0.5 mg/ml in 50 mM citrate buffer, pH=4.6) and incubation for 15 min at 60° C. Glucose was analysed according to the HPAEC method mentioned above.

### Gene Expression Analysis

In order to determine the mRNA abundance of a xylosidase gene putatively involved in the xylose biosynthetic pathway, total RNA was extracted from both flower petals and leaves (from frozen samples).

Total RNA extraction was performed using 1 gram of ground tissue as described by Chang et al “A simple and efficient method for isolating RNA from Pine trees”, Plant Molecular Biology Reporter Volume 11(2), 1993, 113-116, followed by DNase I (AMPD1, Sigma Aldrich) treatment and column purification (RNeasy kit, QIAGEN). Purified RNA was quality checked and quantified by agarose gel electrophoresis and NanoDrop (Thermo Scientific). 200 nanogram of total RNA was inverse transcribed to cDNA (iScript, Biorad), diluted 2.5 times and used for Quantitative Real-Time PCR (qRT-PCR)

For rose xylosidase, a primer set was developed. No rose xylosidase was found in gene databases on the internet but several ESTs were found in the PT OKEE CDNA bank (WUR-FBR private data. The cDNA bank consists of small stretches of rose derived EST sequences cloned in plasmids and maintained in bacteria. The EST constructs were sequenced to determine the EST nucleic acid code. Using sequence homology of the A. thaliana beta xylosidase genes BXL1 to BXL4, three rose ESTs were selected for use in primer design as these EST sequences all seem to be part of the 3′ end of the xylosidase gene: OProseR0396, OProseR0735 and OProseR1560.

The primer set, as well as the primer sets to reference genes (Actinand 18S ribosomal RNA genes), were tested for their efficiency on cDNA of two different rose cultivars (Avalanche and Happy Hour) to see if any variance exists between cultivars. Primer pairs 5′-CAAAGGTCCCGTGGTATTTC-3′ (SEQ ID NO: 1)/5′-GTGGTGGCACTTAGACTTG-3′ (SEQ ID NO: 2) (forward and reverse primer beta xylosidase) and 5′-TGGAGAGTGATTGGGATCTTTT-3′ (SEQ ID NO: 3)/5′-TCCATAGCAGTTTATGACCACA-3′ (SEQ ID NO: 4) (forward and reverse primer Actin) were selected for further qPCR experiments as they show acceptable quality on both cultivars tested.

Each gene expression measurement comprised of 5 μl of cDNA, 2.5 μl forward and reverse primer (concentration dependent on primer efficiency which is 0.4 μM final concentration) and 10 μl IQ SyberGreen Supermix (Biorad) which was real time evaluated for 40 cycles (10″ at 95° C., 10″ at 58° C. and 15″ at 72° C. followed by 2′ and 30″ at 72° C. and 95° C. respectively) followed by a melting curve analysis of 50 cycles (1° C. decrease per cycle starting from 10″ at 95° C.). qRT-PCR relative fold changes were calculated using the 2̂^{-ΔΔCt} method.

### Experiments and Results

### Example 1

### Experimental Parameters

Product: Avalanche roses; source: The Netherlands.
Pretreatment conditions: Chrysal RVB overnight at 4° C.
Storage conditions: Dry in carton flower boxes at 4° C. up till 32 days.
Rehydration conditions: Chrysal RVB for 2 h at room temperature.
Measurements: sugars and starch in outer petals.

### Results:

HPLC chromatograms showed a number of clearly definable peaks that were identified using authentic standards as being glucose, fructose, sucrose, myo-inositol and, in addition, the rare sugar xylose.

Concentrations of both glucose and fructose showed a slight increase over time, levelling off at later time points. Glucose increased from 60 to about 70 mg/gDry Weight (DW); fructose increased from 100 to about 140 mg/gDW. Sucrose (25 mg/gDW) and myo-inositol (6 mg/gDW) did not show a clear change over time. Starch levels in petals at the start of the experiment were low and decreased to zero within 10 days.

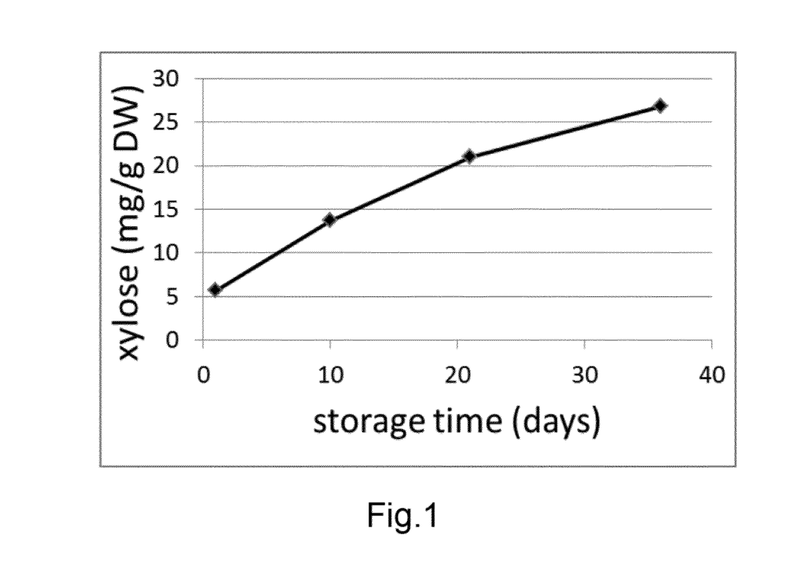

There was a significant increase in xylose levels, from 5 to 27 mg/gDW, in petals with increasing storage time (FIG. 1).

### Example 2

### Experimental Parameters

Product: roses Avalanche, Akito, Happy hour; source: The Netherlands.
Pretreatment conditions: heads were dipped in 200 ppm chlorine solution to prevent botrytis infection.
Storage conditions: dry storage of sleeved bunches in carton boxes for different periods of time at 12° C. (5 days), 5° C. (13 days) and 0.5° C. (22 days).
Rehydration conditions: Chrysal RVB for 2 h at 5° C.
Measurements: sugars in petals; xylosidase mRNA abundance in petals and leaves of selected treatments.

### Results:

### Sugars in Petals

Glucose concentration in petals showed an increasing trend with storage time and this trend was not clearly influenced by the storage temperature. For Avalanche, Akito and Happy Hour, initial glucose levels were 70, 50 and 40 mg/gDW and end levels were 90, 90 and 50 mg/gDW, respectively.

Fructose levels showed a slight increase over time, and levels were little influenced by the temperature. For Avalanche, Akito and Happy Hour initial levels of fructose were 150, 80 and 60 mg/gDW and end levels were 160, 140 and 80 mg/gDW, respectively.

Sucrose concentrations showed a slight decreasing trend in Avalanche (from 45 to 35 mg/gDW) which was not influenced by temperature. In Akito and Happy Hour sucrose slightly decreased during storage at 12° C. and 5° C. (from 25 to 15 mg/gDW in Akito; from 30 to 20 mg/gDW in Happy Hour) but sucrose increased during storage at 0.5° C. (from 25 to 35 mg/gDW in Akito; from 30 to 40 mg/gDW in Happy Hour).

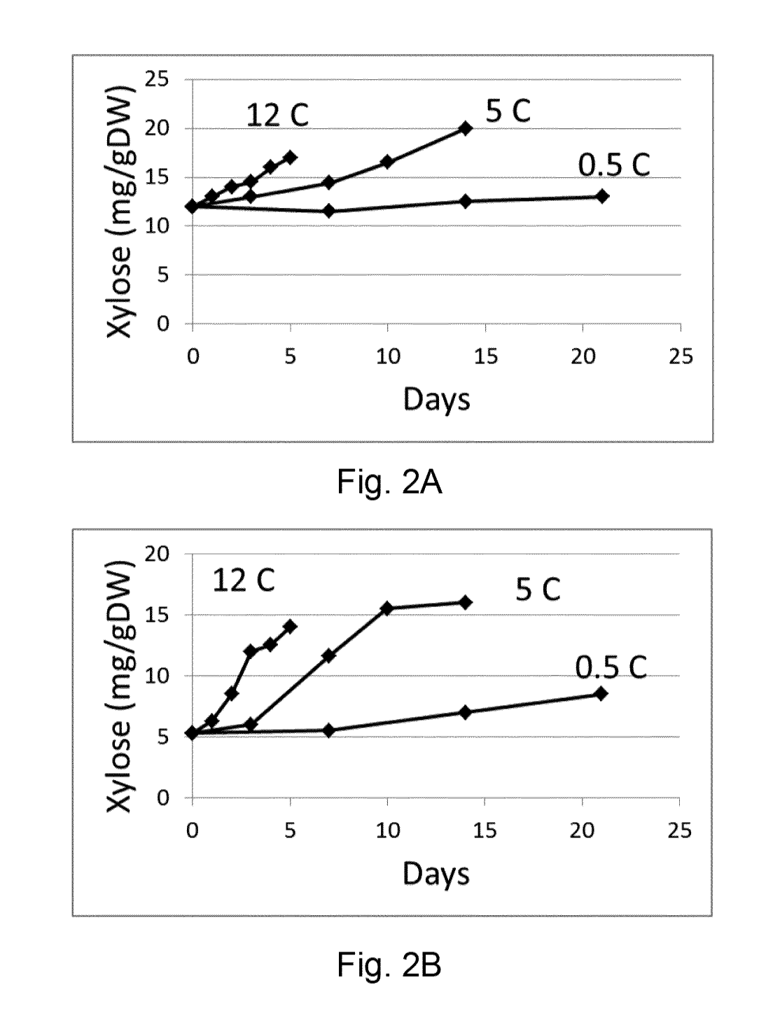

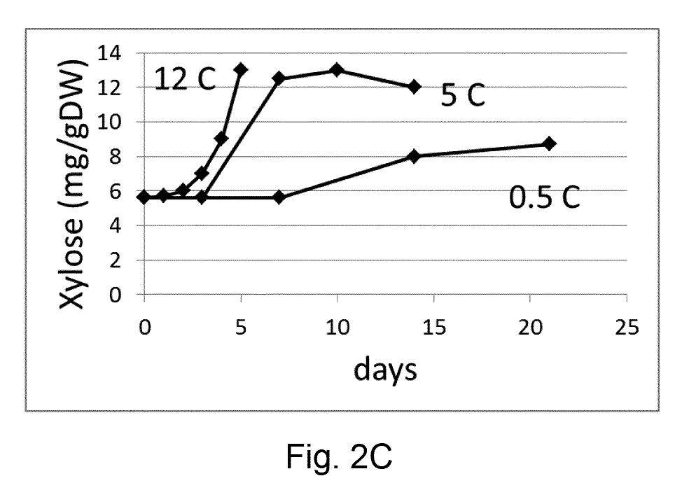

Xylose concentration in petals increased in all cultivars under all storage conditions (FIG. 2). The initial level of xylose in Avalanche (11 mg/gDW, panel A) was higher than in Akito (panel B) and Happy Hour (panel C) (both 5 mg/gDW). Depending on the temperature, a faster or slower increase in xylose was observed (FIG. 2). Although the duration of the storage was not enough to detect end levels of xylose, judging from the graphs, the end levels are expected to be dependent on the temperature. At lower storage temperature, the increase in xylose is slower and the end level is expected to be lower than at higher storage temperature.

β-Xylosidase mRNA Abundance in Petals and Leaves

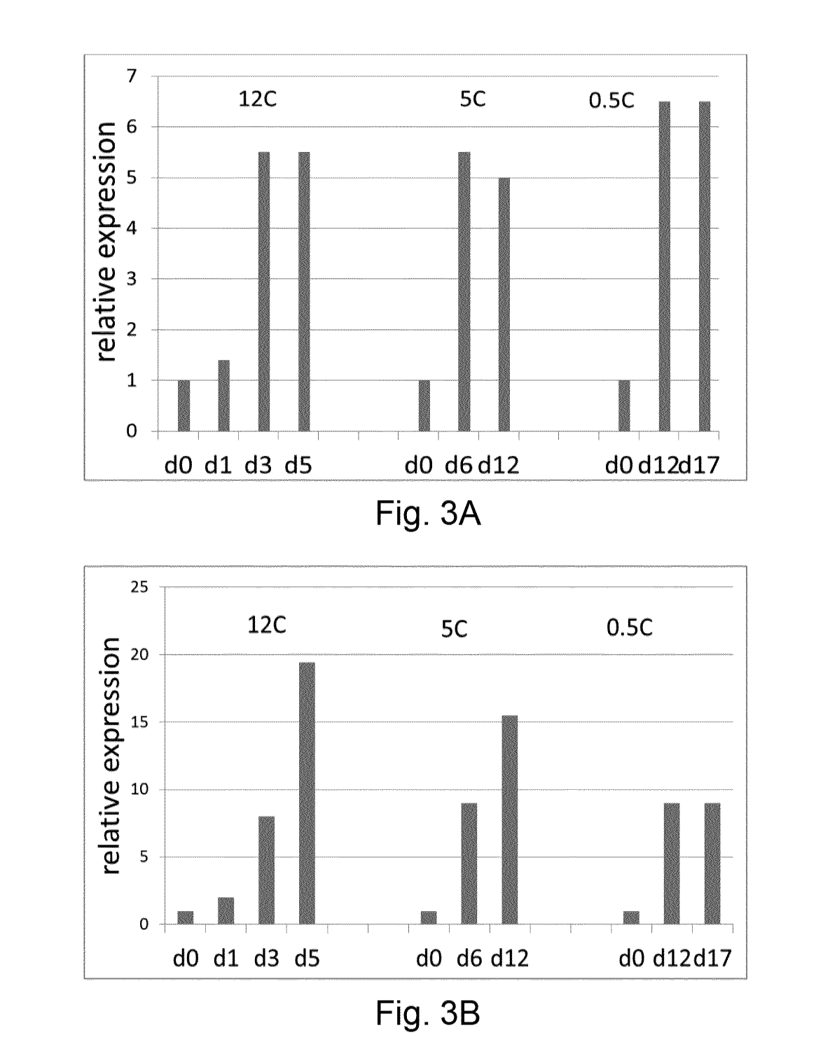

β-xylosidase mRNA abundance was measured in outer petals from cv. Avalanche (FIG. 3 panel A) and from cv. Happy Hour flowers (FIG. 3 panel B) stored for different periods at different temperatures (FIG. 3). Relative expression showed a clear increase up to 5-6 times the initial level in Avalanche and up to 15-20 times the initial level in happy hour. In cv. Avalanche the end level was little influenced by the storage temperature; in cv. Happy Hour the end level seemed dependent on temperature, being higher at higher storage temperature.

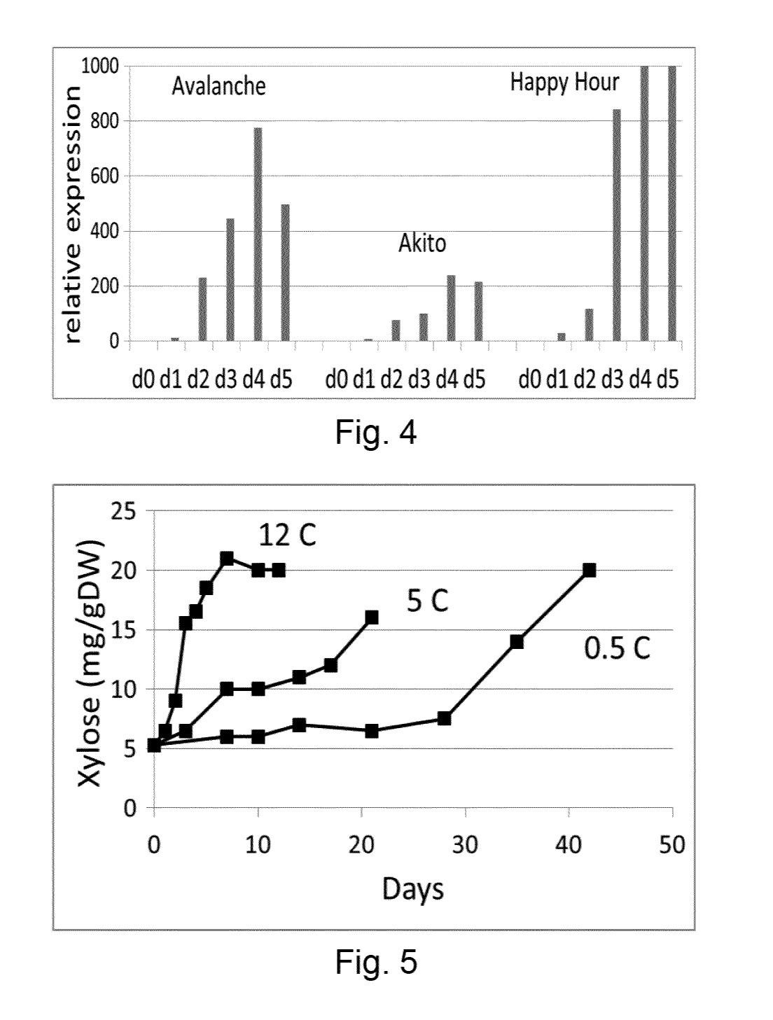

β-xylosidase mRNA abundance was also measured in leaves from cv. Avalanche, cv. Happy Hour and cv. Akito roses following storage at 12° C. for various periods of time (FIG. 4). Within 2 to 3 days of storage, the mRNA levels were significantly increased in all three cultivars. End levels were cultivar dependent, amounting to 100, 600 and more than 1000 times the initial levels in cultivars. Akito, Avalanche and Happy Hour respectively.

### Example 3

### Experimental Parameters

Product: rose cv. Akito; source: The Netherlands
Pretreatment conditions: heads were dipped in 100 ppm chlorine solution to prevent botrytis infection
Storage conditions: dry storage of sleeved bunches in carton boxes for different periods of time at 12° C. (maximum 12 days), 5° C. (maximum 21 days) and 0.5° C. (maximum 42 days)
Rehydration conditions: Chrysal RVB for 2 h at 5° C.
Measurements: sugars (glucose, fructose, sucrose, myo-inositol, methyl-β-D-Glucopyranoside, and xylose) in outer petals and leaves (the tip and 2 outer small leaflets, closest to the tip leaflet from the first or second foliate leaf complex under the flower head); xylosidase mRNA abundance in petals and leaves of selected treatments

### Results:

Sugars in Petals of Roses cv. Akito

Glucose and fructose concentrations in petals showed an increasing trend with storage time and this trend was not clearly influenced by the storage temperature. Initial glucose level was 55 mg/gDW and end level was approximately 70 mg/gDW; initial fructose level was 70 mg/gDW and end level was 120 mg/gDW. Sucrose levels slightly decreased during storage at 12° C. and 5° C. (from 20 to 15 mg/gDW, but sucrose increased at 0.5° C. (from 20 to 25 mg/gDW).

Myo-inositol concentration was approximately 7 mg/gDW and the level was not affected by storage, irrespective of the temperature. Methyl-β-D-Glucopyranoside showed a slight increase during storage from 6 to 8 mg/gDW. The level was not influenced by the storage temperature.

Xylose concentrations in petals showed an increase during storage and the speed of the increase was dependent on the storage temperature. Initial level of xylose was 5 mg/gDW and end levels amounted to approximately 23, 15 and 20 mg/gDW at 12° C., 5° C. and 0.5° C., respectively (FIG. 5).

Sugars in Leaves of Roses cv. Akito

Glucose concentration in leaves showed an increasing trend with storage time and this trend was influenced by the storage temperature. Initial glucose level was 5 mg/gDW and the end level was approximately 20, 20 and 15 mg/gDW at 12° C., 5° C. and 0.5° C., respectively.

Initial fructose level was 8 mg/gDW and the levels showed an increase during storage at 12° C. (up to 23 mg/gDW) whereas levels decreased to zero within 15 days of storage at 5° C. and 0.5° C.

Sucrose levels decreased during storage and this decrease was little influenced by the storage temperature. Initial sucrose level was 350 mg/gDW and end level was approximately 40-50 mg/gDW.

Myo-inositol concentration was approximately 80 mg/gDW and slightly increased to 90 mg/gDW during the first 5-10 days of storage. The increase was not affected by storage temperature. Methyl-β-D-Glucopyranoside in leaves was below the detection level (<0.5 mg/gDW) throughout the storage period.

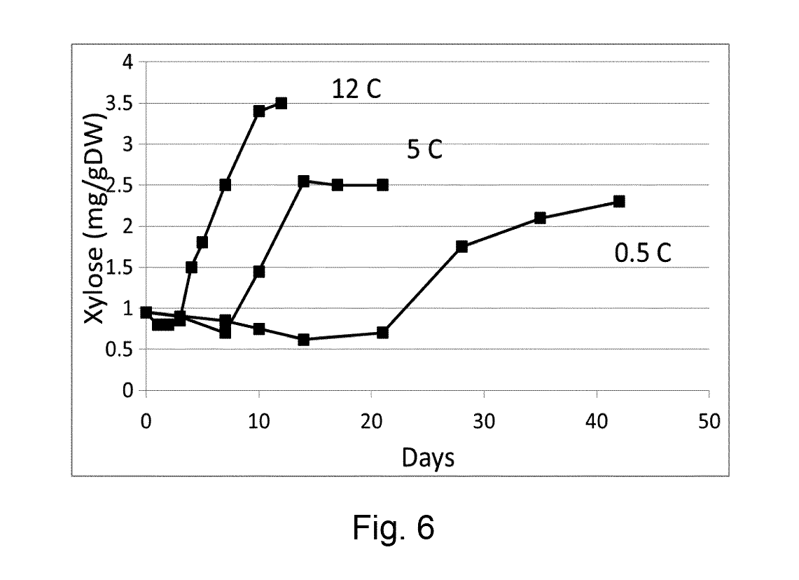

Xylose concentrations in leaves showed first a minor decrease and thereafter an increase during storage; the speed of the increase was dependent on the storage temperature. Initial level of xylose was 1 mg/gDW and end levels amounted to approximately 3.5, 2.5 and 2.5 mg/gDW at 12° C., 5° C. and 0.5° C., respectively (FIG. 6).

mRNA Abundance in Petals and Leaves of Roses cv. Akito

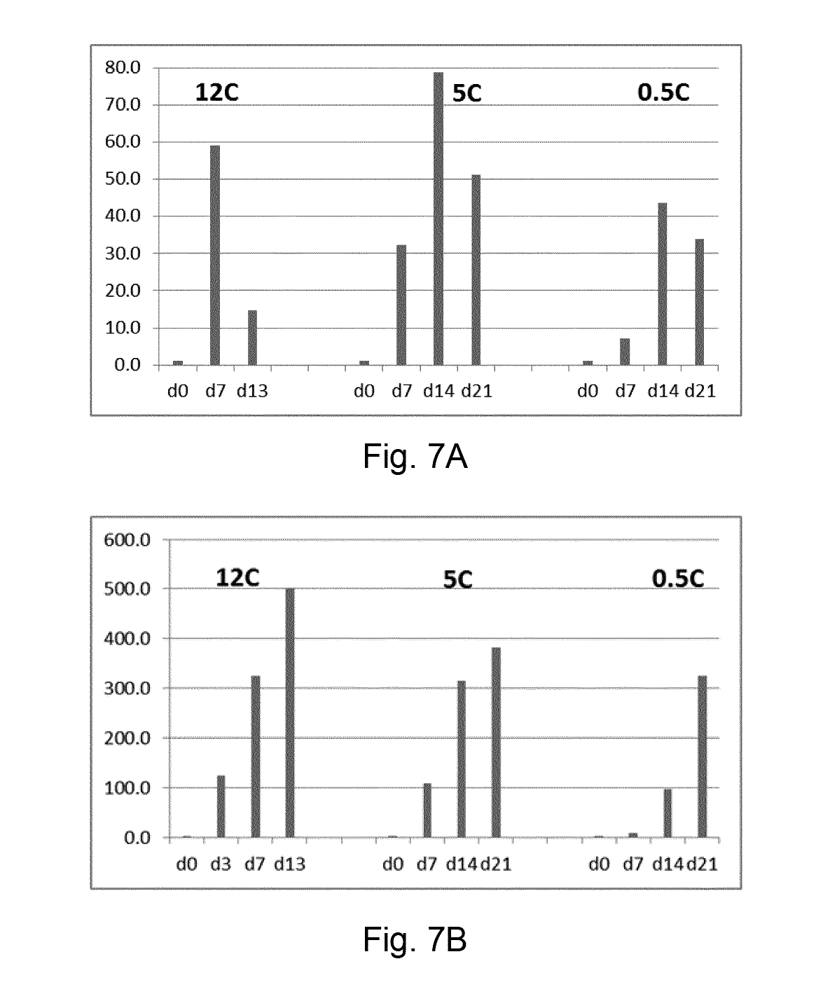

Beta xylosidase mRNA abundance in the petals increased during 14 days of storage, independent of storage temperature except for 12° C. After 14 days the expression started to decrease as shown for day 21 for 5° C. and 0.5° C. (FIG. 7A). This decrease after longer periods of storage time was temperature dependent with a more rapid decrease for higher temperatures than lower temperatures. Relative expression reached a maximum of up to 80 times the initial level at 5° C. and up to 60 and 40 times the initial level at 12° C. and 0.5° C. respectively.

In the leaves, the relative expression of beta xylosidase kept increasing during storage time without the decrease after longer storage time seen for the petals (FIG. 7B). The maximum relative expression was temperature dependent with the highest and most rapid increase for 12° C. (500 times for 12° C. versus 380 and 325 for 5° C. and 0.5° C. respectively)

### Example 4

### Experimental Parameters

Product: rose cv. Red Naomi; source: The Netherlands
Hydration: 2 h in water at 20° C.
Storage conditions: dry storage of sleeved bunches of 10 flowers each in carton boxes for different periods of time at 12° C. (maximum of 19 days), 8° C. (maximum of 19 days), 5° C. (maximum of 33 days) and 0.5° C. (37 days)
Rehydration conditions: After storage, flowers were recut and placed in water for 2 h at 5° C.
Measurements: sugars (glucose, fructose, sucrose, myo-inositol, methyl-β-D-Glucopyranoside, and xylose) in petals and leaves; β-xylosidase mRNA abundance in petals and leaves.
Vase life determination: the vase life was tested of flowers placed in: water+50 ppm HQS; and in: water+50 ppm HQS+1% sucrose.

### Results

Sugars in Petals of Roses cv. Red Naomi

For sugar extraction a slightly different method was used than in the other experiments. In this case, extracts were made from frozen material, without freeze drying. For clearness, all values have been re-calculated to mg/gDW, assuming that the tissue dry weight is approximately 6% of the fresh weight.

Glucose in petals showed an increase from 30 to 40 mg/gDW during storage at 0.5° C., 5° C. and 8° C.; whereas a small decrease was observed at 12° C. storage temperature (from 30 to 25 mg/gDW). Fructose concentrations in petals showed an increasing trend (from 50 to about 75 mg/gDW) with storage time and this trend was not clearly influenced by the storage temperature. Sucrose levels were stable during storage at 0.5° C. (25 mg/gDW) but decreased during storage at 5° C. (from 25 to 15 mg/gDW), 8° C. (from 25 to 12 mg/gDW) and 12° C. (from 25 to 10 mg/gDW).

Myo-inositol concentration was stable at 0.5° C., 5° C. and 8° C. storage (level approximately 6.5 mg/gDW); at 12° C. a slight decrease was observed (from 6.5 to 4 mg/gDW).

Methyl-β-D-Glucopyranoside level was approximately 7 mg/gDW; the level did not change during storage and was not influenced by the storage temperature.

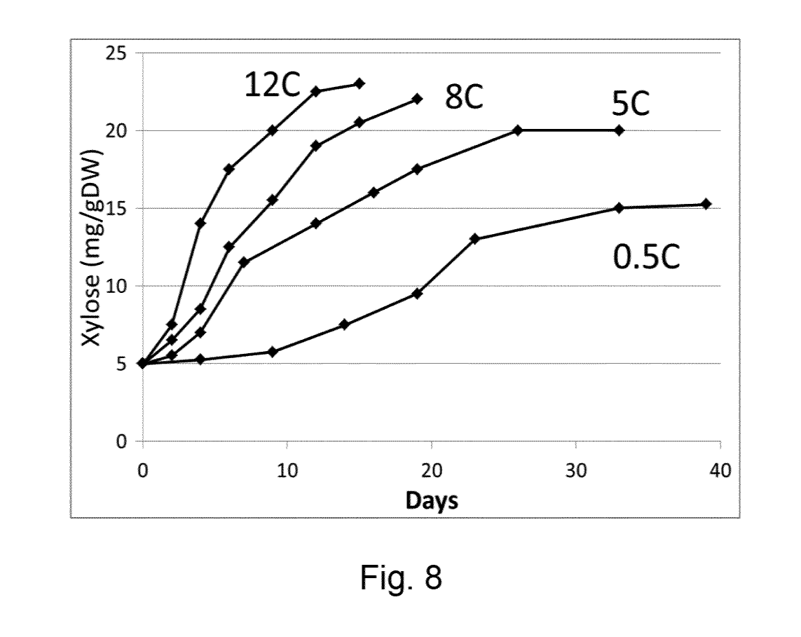

Xylose concentrations in petals showed an increase during storage and the speed of the increase was dependent on the storage temperature. Initial level of xylose was 5 mg/gDW and end levels amounted to approximately 24, 22, 20 and 15 mg/gDW at 12° C., 8° C., 5° C. and 0.5° C., respectively (FIG. 8).

Sugars in Leaves of Roses cv. Red Naomi

Glucose in leaves was about 2.5 mg/gDW. The glucose level did not change during storage at non of the applied temperatures. Fructose concentration in leaves was about 2.5 mg/gDW at the start of the storage and showed a temperature dependent decrease to almost zero. The decrease in fructose concentration was faster when the storage temperature was higher. Sucrose levels in leaves decreased during storage from 70 to about 10 mg/gDW and this decrease was independent of the temperature.

Myo-inositol concentration was about 40 mg/gDW at the start of the storage and decreased to 30, 25, 20 and 12.5 mg/gDW at 0.5° C., 5° C., 8° C. and 12° C. storage.

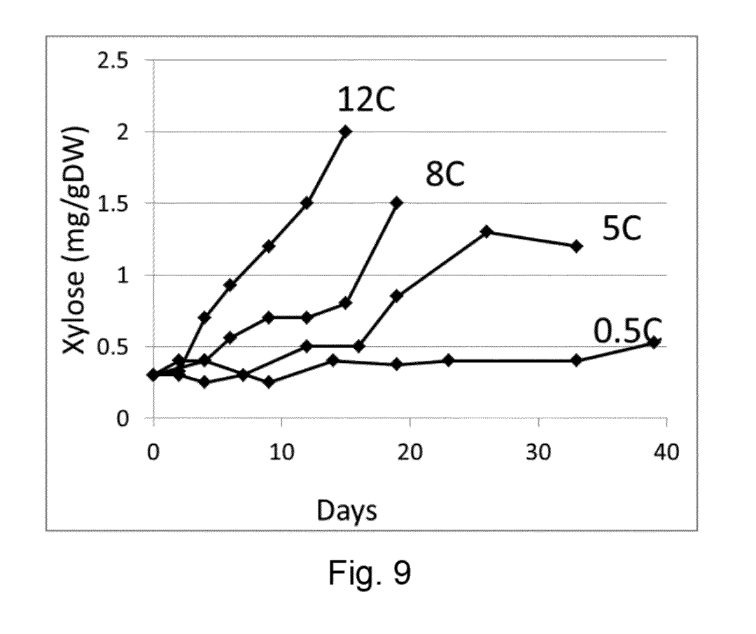

Xylose concentrations in leaves showed an increase during storage and the speed of the increase was dependent on the storage temperature. Initial level of xylose was 0.3 mg/gDW and end levels amounted to approximately 2, 1.5, 1.3, and 0.5 mg/gDW at 12° C., 8° C., 5° C. and 0.5° C., respectively (FIG. 9).

### Correlation Between Xylose Concentration and Vase Life

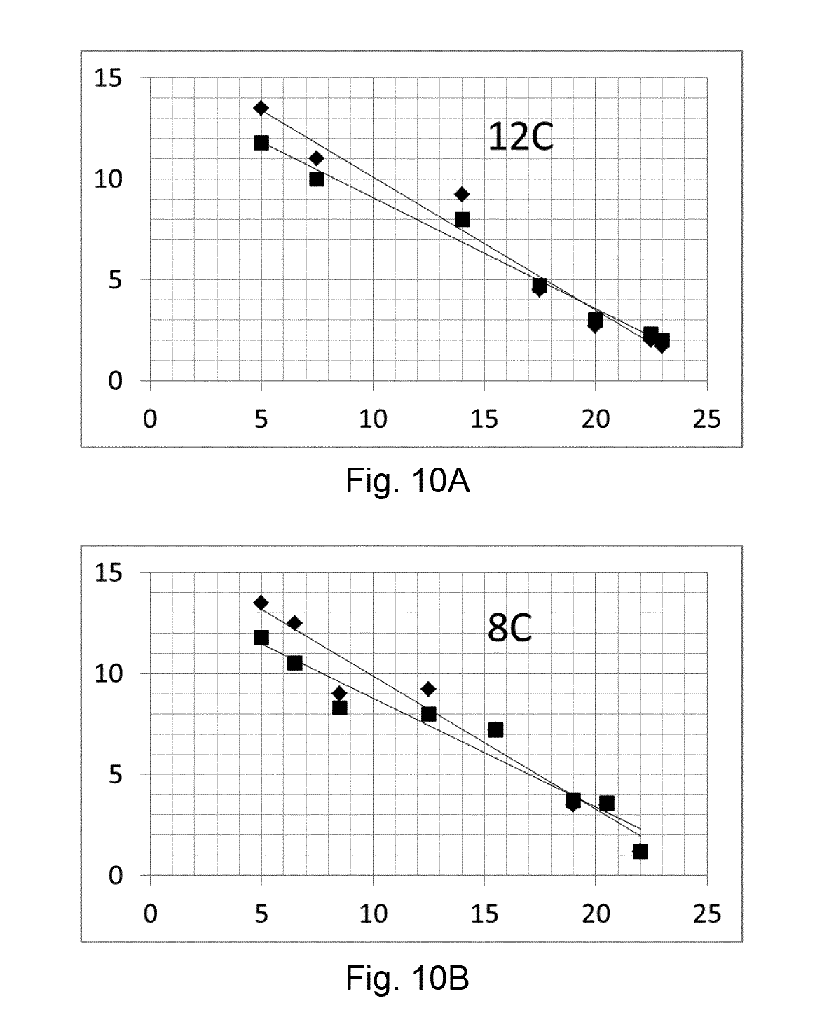

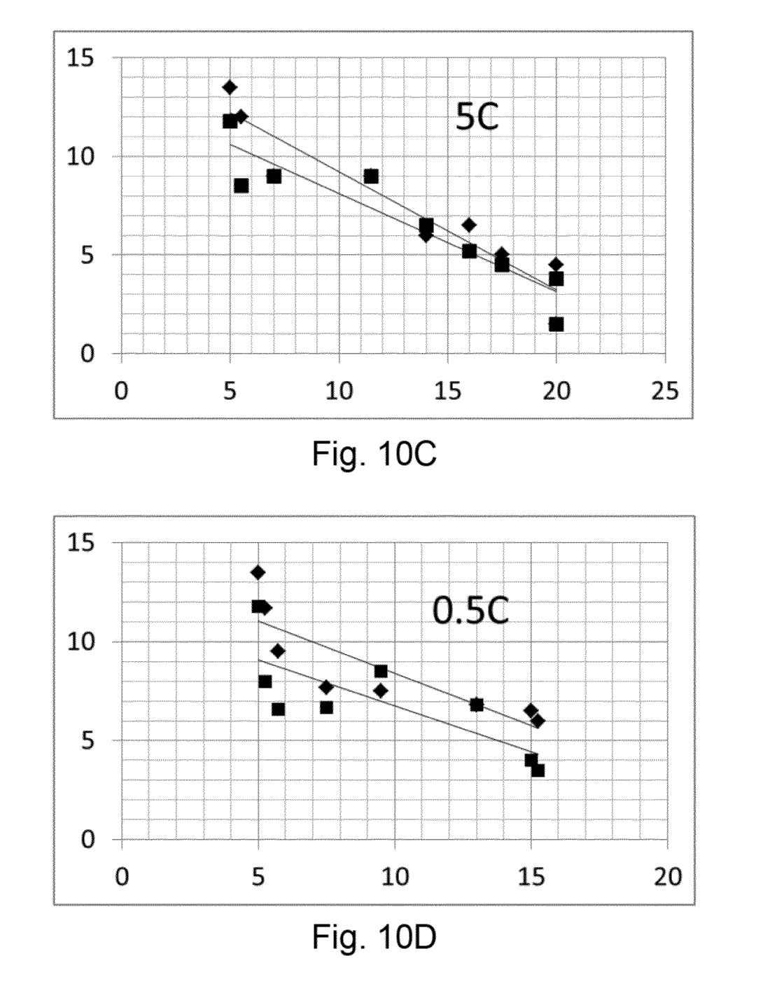

In general, the vase life of the flowers was shorter after longer storage time and after storage at higher temperatures. The correlation between the xylose concentration after storage and the corresponding vase life for two different groups of flowers (vase life in water+HQC and vase life in sucrose+HQC) is shown in FIG. 10. The combination of the different storage temperatures for the two different vase life conditions and the combination of the two vase life conditions is shown in FIG. 11.

This shows that there is an overall good correlation between the level of xylose measured in the outer petals and the corresponding vase life of the particular group of flowers over the whole range of storage temperatures and storage durations investigated in this experiment.

β-Xylosidase Gene Expression in Petals of Roses cv. Red Naomi

B-xylosidase relative mRNA abundance showed an increase with increasing storage duration at all storage temperatures, amounting up to 250 times the initial level (FIG. 12). The pattern of gene expression was little influenced by the temperature, except for storage at 12° C. Within 2 days of storage, expression was increased by 5 to 10 times at all temperatures. At longer storage times, especially at 12° C. expression decreased again.

β-Xylosidase Gene Expression in Leaves of Roses cv. Red Naomi

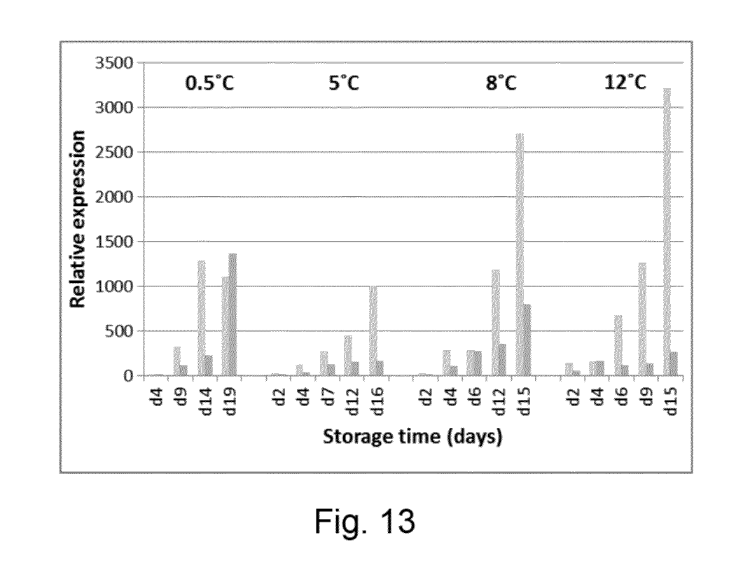

β-xylosidase mRNA abundance as measured in the leaves increased to much higher levels of relative expression than seen for in the petals and amounting up to 3200 times the initial level (FIG. 13). There was a high variation in relative gene abundance between the two biological duplicate samples but the pattern of gene expression was similar at all temperatures. The gene expression increased with storage time with a higher increase for the higher storage temperatures. The decrease of beta xylosidase expression at longer storage times as seen in the petals was absence in the leaves.

### Example 5

### Experimental Parameters

Product: rose cultivars. Akito, Red Naomi, Sphinx Gold, Passion, Aqua; source: The Netherlands
Pretreatment conditions: 4 h in cold water (8° C.)
Storage conditions: dry storage of sleeved bunches in carton boxes for 12 days at 8° C.
After storage the flowers were recut and rehydrated in cold tap water for 2 hours at 8° C. Measurements: sugars (glucose, fructose, sucrose, myo-inositol, methyl-β-D-Glucopyranoside and xylose) in outer petals
Vase life: in Chrysal Professional 3.

### Results

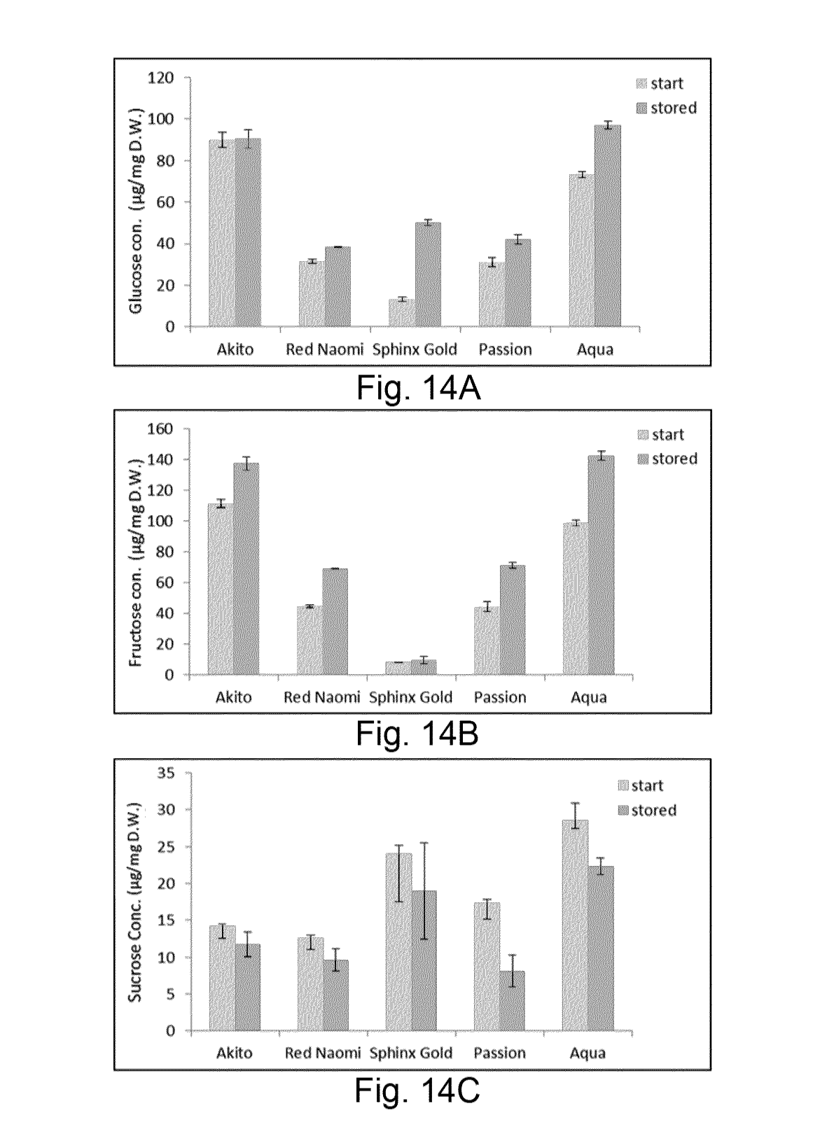

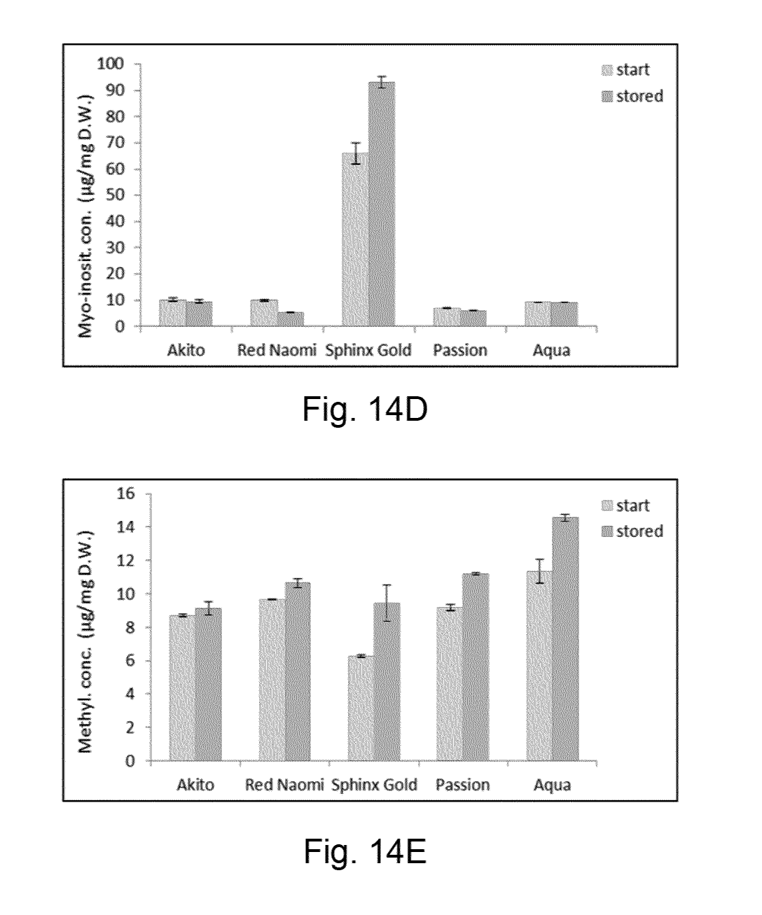

Levels of different sugars before and after 12 days of storage at 8° C. are shown in FIG. 14. Glucose and fructose varied between cultivars but generally showed an increase after storage compared to the initial levels. Sucrose levels were lower than glucose and fructose levels and decreased during storage.

Except for cv. Sphinx Gold, initial myo-inositol concentration was low (about 8 mg/gDW) and relatively stable during storage. In Sphinx Gold myo-inositol concentration was exceptionally high and increasing during storage.

Methyl-β-D-Glucopyranoside was around 8 mg/gDW and did not show consistent change during storage.

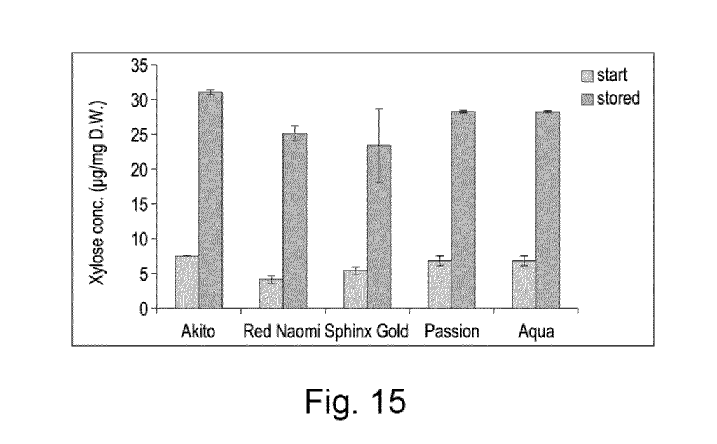

Xylose concentration was low (around 6 mg/gDW) in petal of all cultivars. before storage. After storage it increased by about 5 times in all cultivars. (FIG. 15).

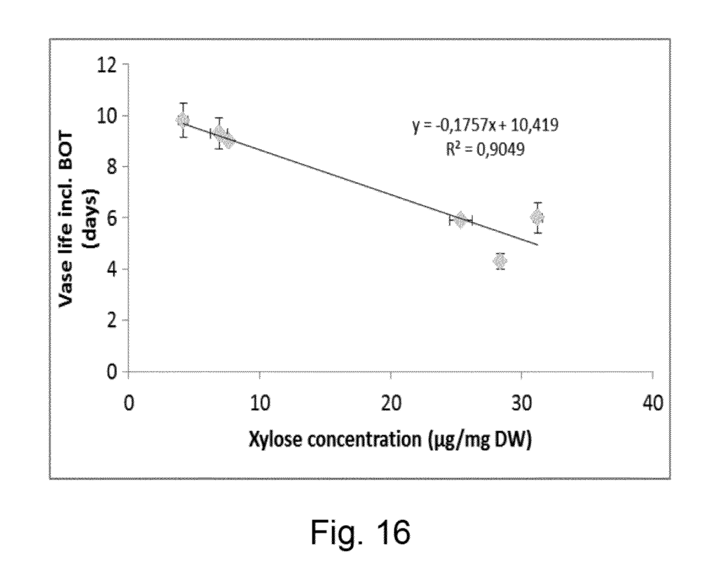

A good correlation exists between the xylose concentration in petals and the vase life of selected cultivars (Akito, Red Naomi and Passion) (FIG. 16).

### Example 6

### Experimental Parameters

Product: rose cultivars. Grand Prix and Avalanche; source: The Netherlands
Storage conditions: dry storage of sleeved bunches in carton boxes for 21 days at 0.5° C.
Rehydration conditions: recut and placed in water at room temperature
Measurements: sugars (glucose, fructose, sucrose, myo-inositol, methyl-β-D-Glucopyranoside and xylose) in outer petals

### Results:

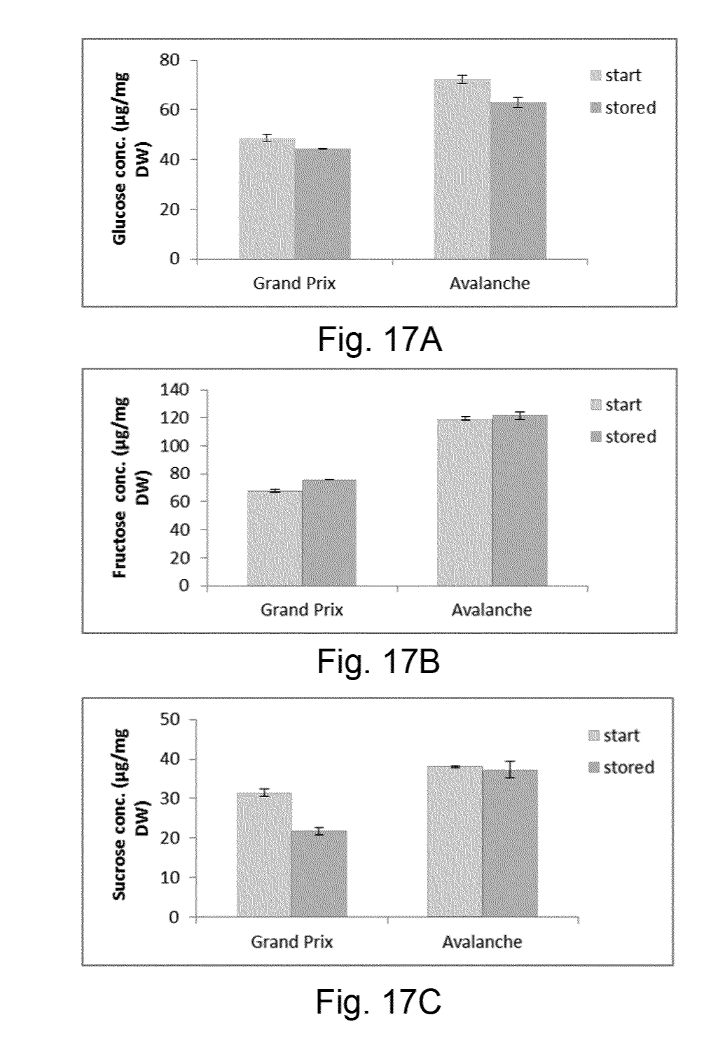

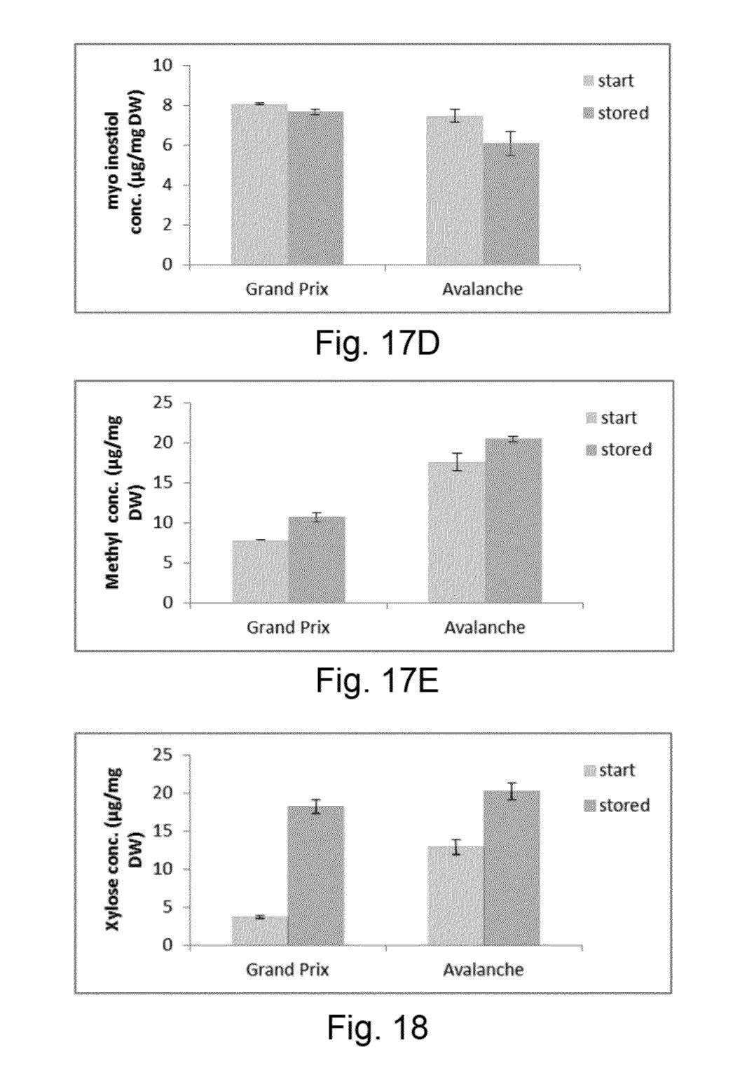

In both cultivars, glucose, sucrose and myo-inositol levels were slightly decreased after 21 days storage at 0.5° C. Fructose and methyl-β-D-Glucopyranoside levels were slightly increased (FIG. 17).

Xylose level in cv. Grand Prix was initially low (about 3 mg/gDW) and showed a 5 times increase during storage. Xylose level in cv. Avalanche was relatively high at the start of the experiment (13 mg/gDW) and showed a relatively minor increase during storage (FIG. 18).

### Example 7

### Experimental Parameters

Product: rose Esperance and Blush; source: Ecuador

Transported to the Netherlands by plane, the roses were packed in cardboard sleeves/collars in cardboard boxes. From the grower in Ecuador to the lab in Wageningen the average temperature was 10° C., for 3 days and 3 hours, the temperature sum (° C.*days) was 31. At that moment the first samples were taken and the flowers were placed in vases. Then a part of the flowers, including the cardbox sleeves were packed in plastic crates and transported by truck from The Netherlands to Germany and back, for nearly 4 days. The average temperature during this truck transport was 9.3° C. After this transport, samples were taken and flowers were placed in vases. The temperature sum of the trip to Germany and back was 36, so the total temperature sum from the grower in Ecuador via Wageningen and Germany back to Wageningen was 67.

Measurements: sugars (glucose, fructose, sucrose, myo-inositol, methyl-β-Dglucopyranoside and xylose) in outer petals at arrival in The Netherlands (after air transport) and after the trip by tuck to Germany and back (4 days).

### Results:

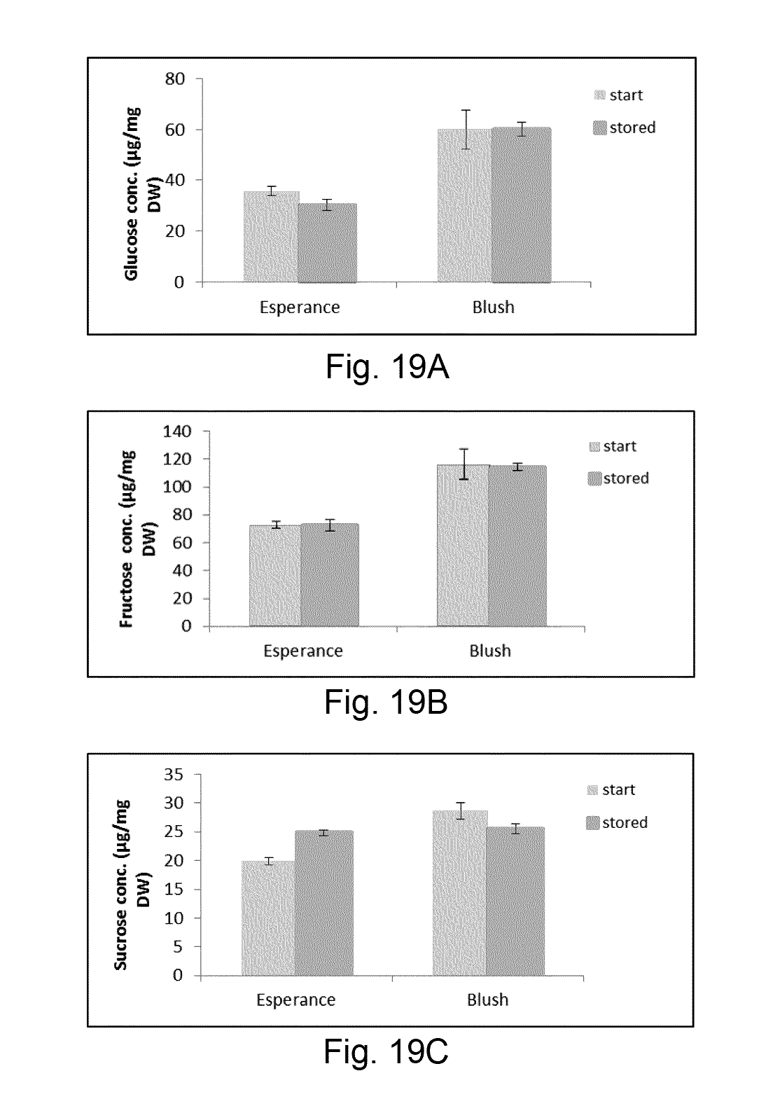

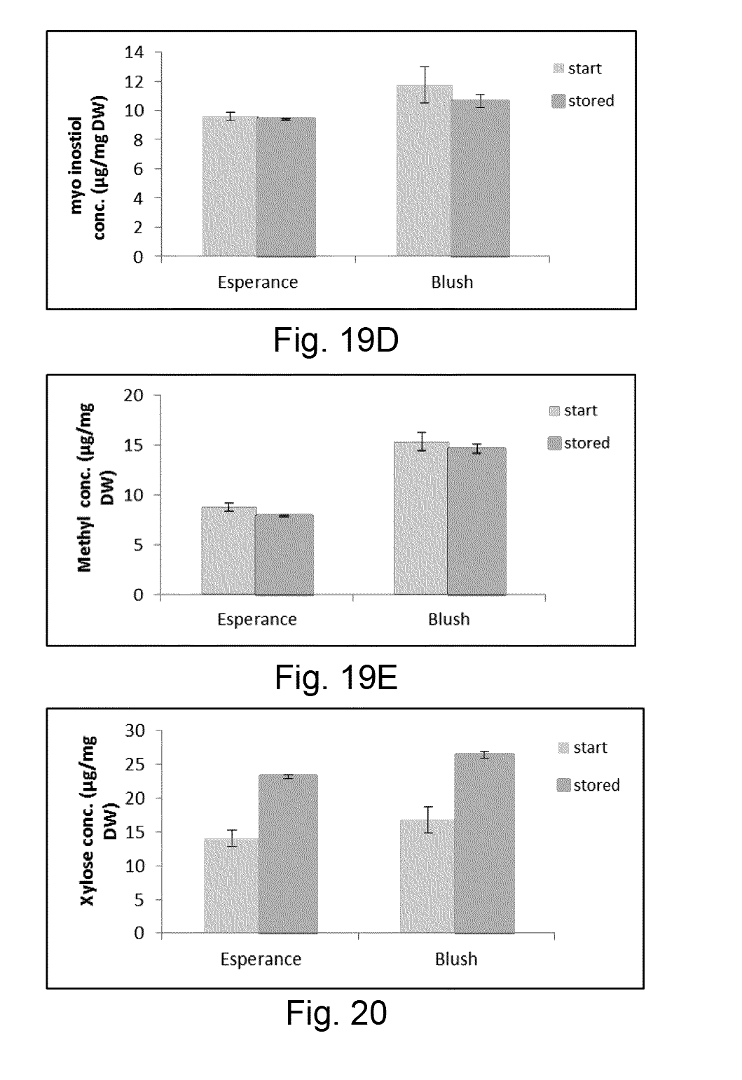

Levels of all sugars were higher in cv. Blush than in cv. Esperance. During the 3 day truck ride, minor changes appeared in the levels of most sugars (FIG. 19). Initial levels of xylose were around 15 mg/gDW in both cultivars. After the 4 day truck ride, levels were increased to about 25 mg/gDW (FIG. 20)

### Example 8

### Experimental Parameters

Product: rose Aqua and Passion; source: The Netherlands

### Pretreatment: 1 day in Chrysal RVB at 1° C.

Distribution simulation:
1 day dry at 5° C., 4 days dry at 8° C., 2 days in water at 20° C. Total storage time 7 days (designated “Stored 1”)
1 day water at 5° C., 4 days dry at 8° C., 2 days in water at 20° C. Total storage time 7 days (designated “Stored 2”)
   Measurements: sugars (glucose, fructose, sucrose, myo-inositol, methyl-β-D-glucopyranoside, and xylose) in outer petals at start of the experiment and after storage.
   Vase life: vase life was determined after the transport simulation; flowers were in Chrysal Professional 3 during vase life.

### Results:

Glucose and fructose levels in both cultivars slightly increased and sucrose decreased during the 7 day distribution period. Myo-inositol concentration was relatively stable, methyl-β-D-glucopyranoside showed an increase during distribution period (FIG. 19).

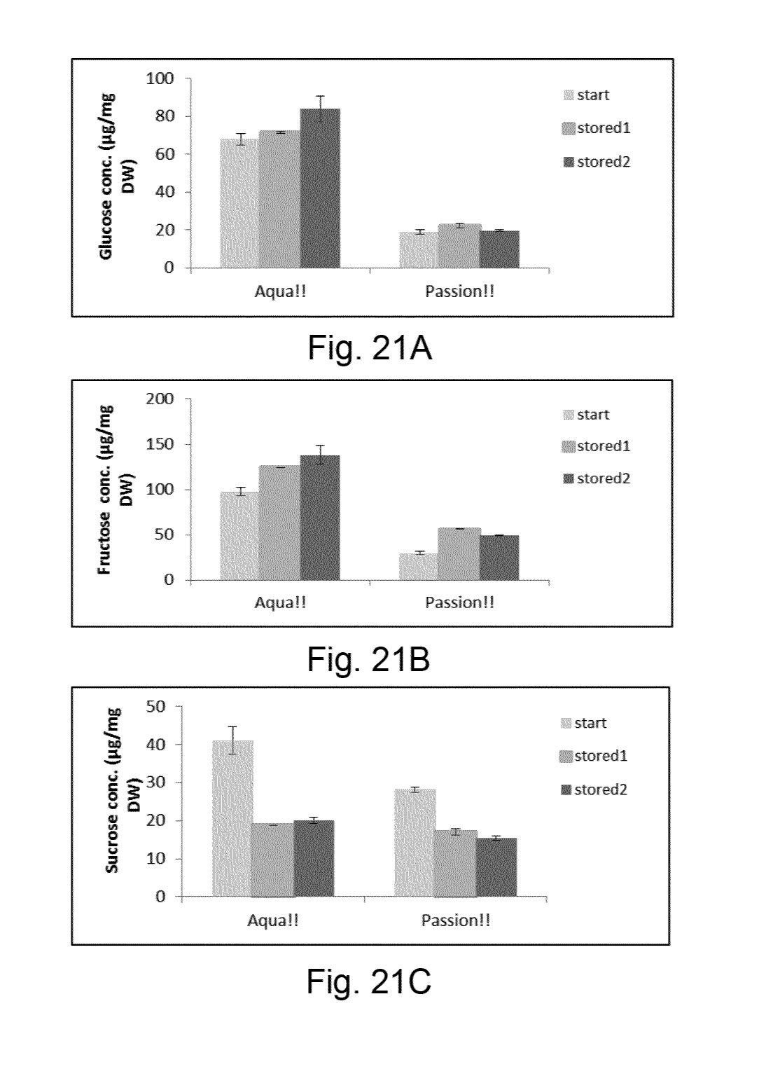

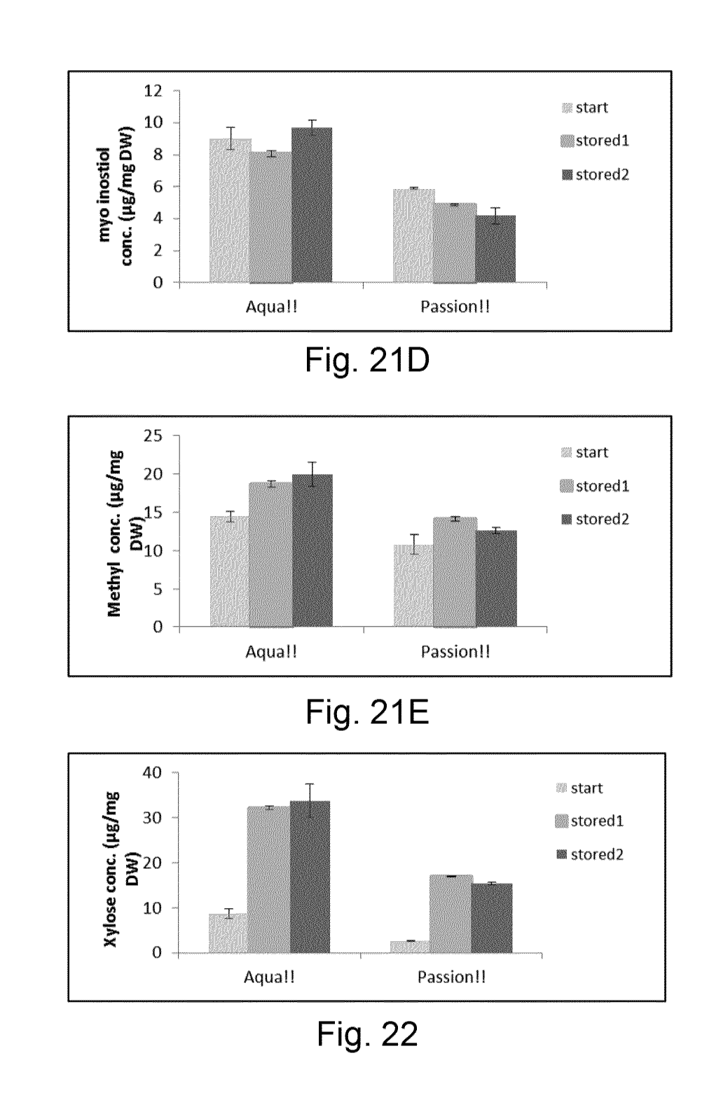

Xylose levels in petals showed a clear increase (about 4 times) during the distribution period (FIGS. 21 and 22).

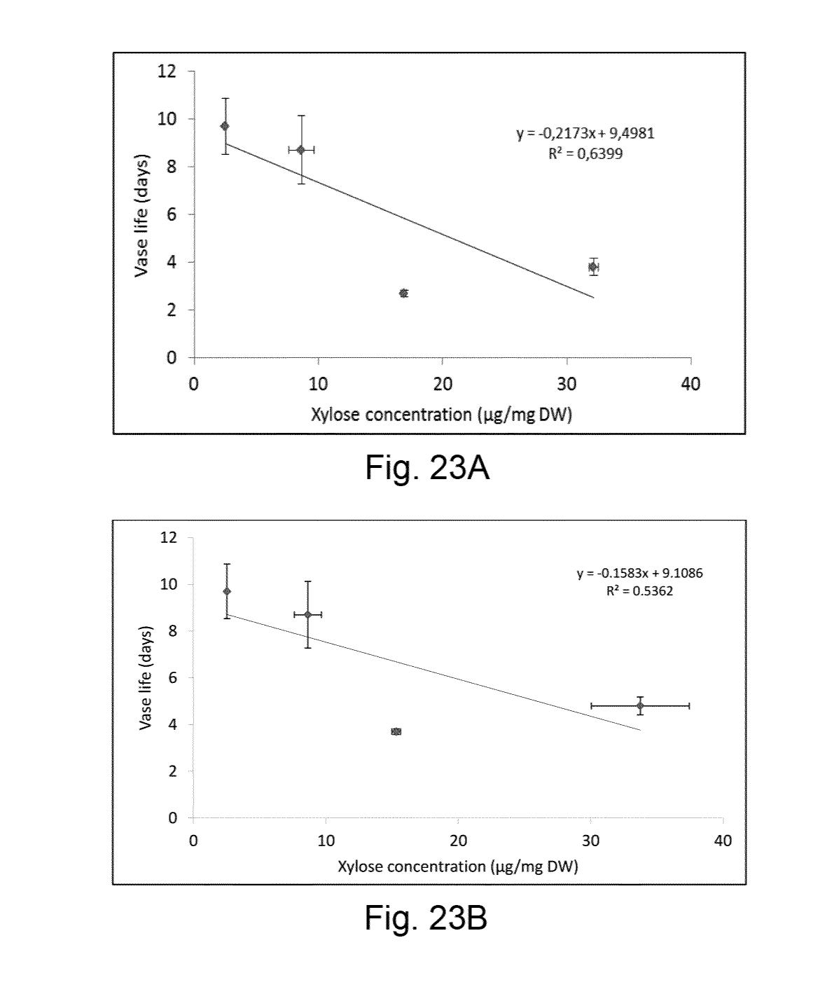

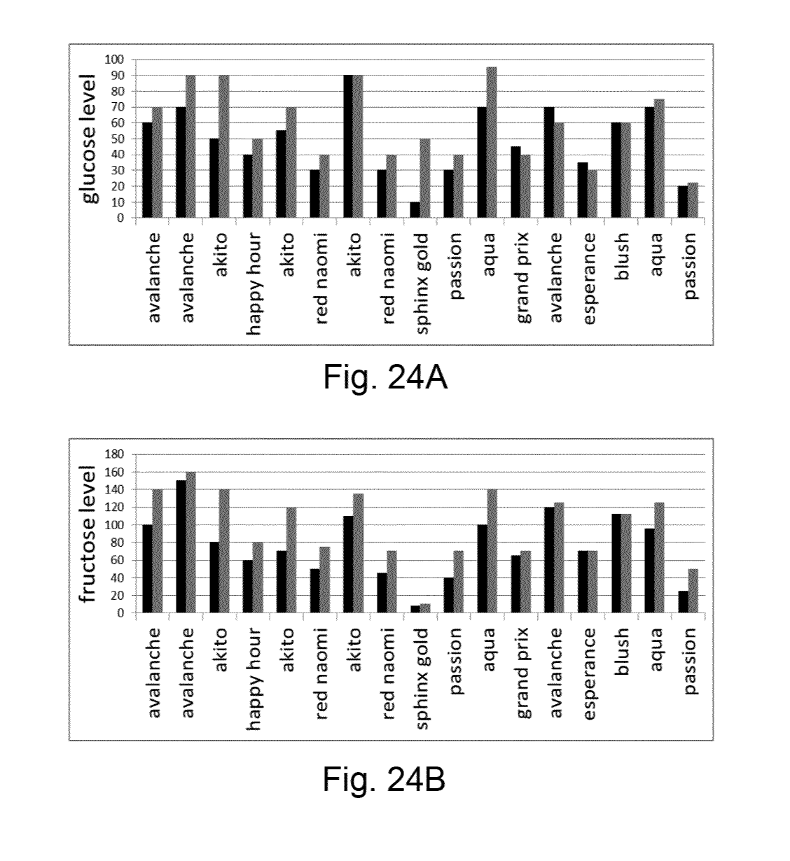

There was a reasonable correlation between the xylose levels at the end of the distribution simulation and the vase life of these groups of flowers (FIG. 23).

### Discussion

### Changes in Sugar Levels

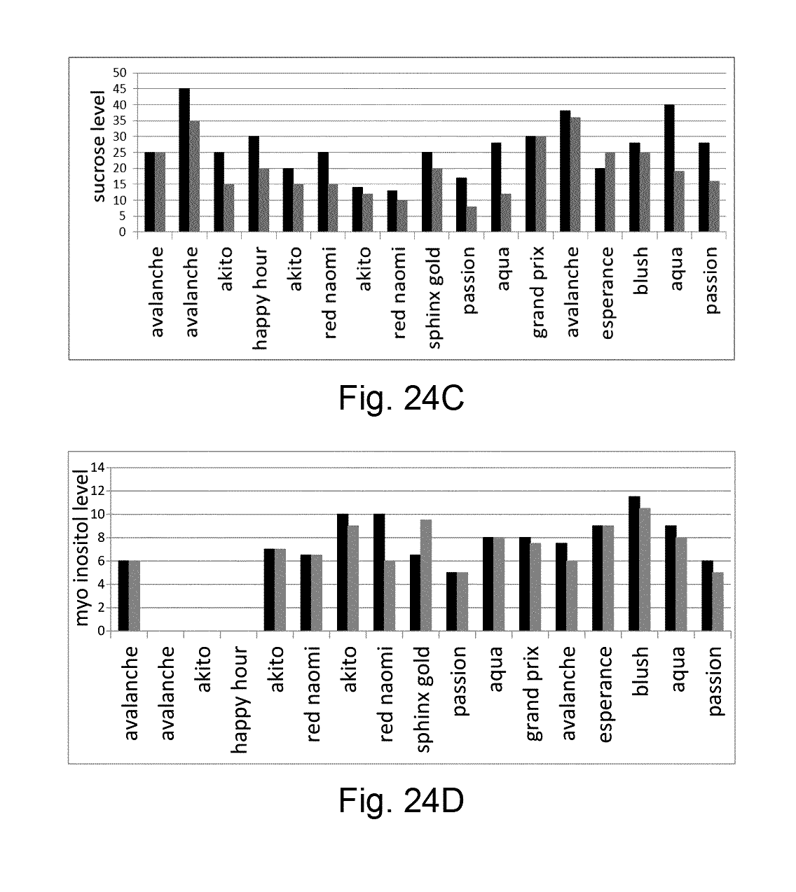

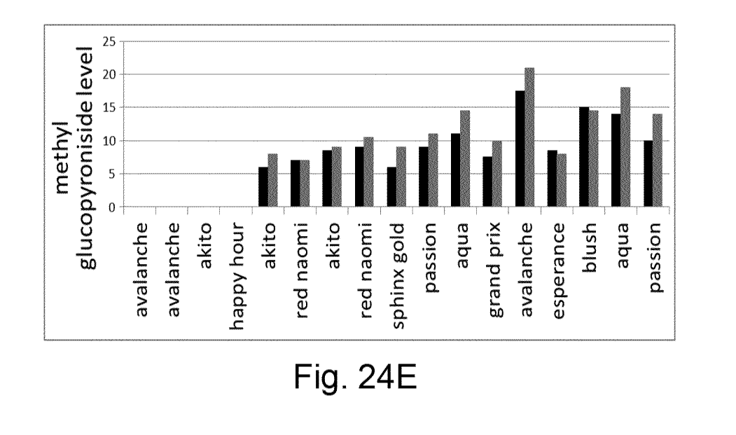

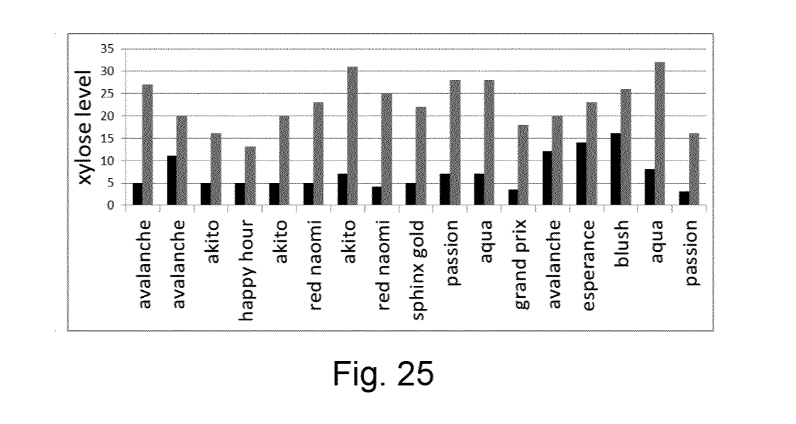

The results of the various experiments are summarized in FIGS. 24 and 25. In these Figures, the starting level of a particular metabolite (glucose, fructose, sucrose, myo-inositol and methyl-β-D-glucopyranoside in FIG. 24; xylose in FIG. 25) in a range of cultivars is compared to the (estimated) level after a substantial storage period. In most of the cultivars, glucose and sucrose levels showed a small increase during storage, whereas sucrose generally showed a decline. The absolute decline in sucrose level was by far not sufficient to explain the increases in reducing sugars, which indicates that during storage sugars were produced from stored polysaccharides such as starch.

Myo-inositol concentration was low in all cultivars (about 6-8 mg/gDW) and the storage generally did not greatly affect this level. The start level of methyl-β-D-glucopyranoside was between 5 and 10 mg/gDW and, showed a slight increase in most cultivars.

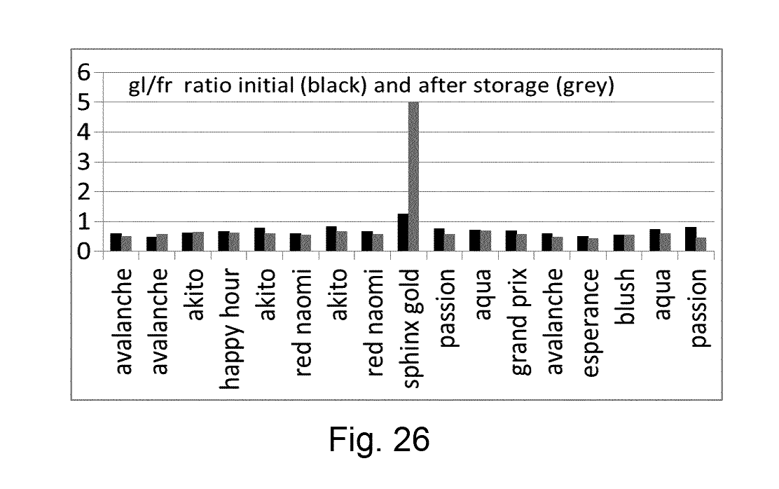

The start level of xylose generally was about 5 mg/gDW, with some exceptions (FIG. 25). Especially in cultivars. where the start sample was taken after prior transportation, the level was higher. End levels were 3 to 5 times higher than start levels, depending on the cultivar and the storage conditions. The initial glucose/fructose ratio generally was about 0.5 to 0.6, and in most of the cultivars the ratio did not change very much during storage (FIG. 26). A clear exception was observed in cultivar sphinx gold where the initial ratio was about 1.2 with a huge change to 5 during storage.

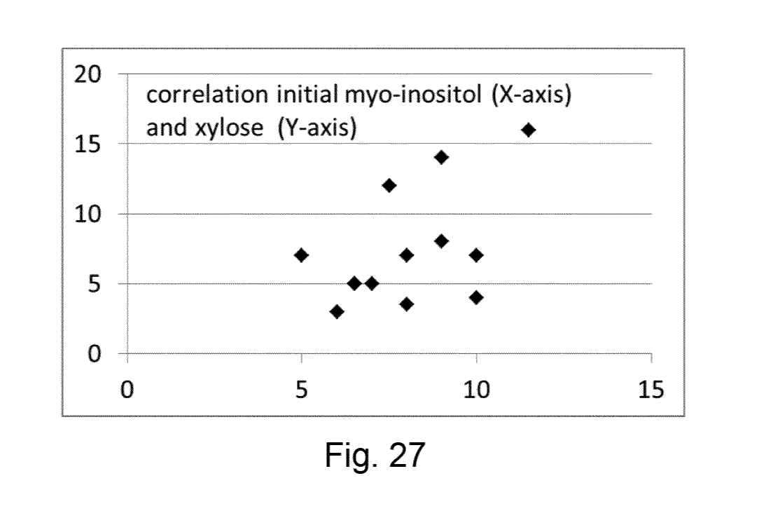

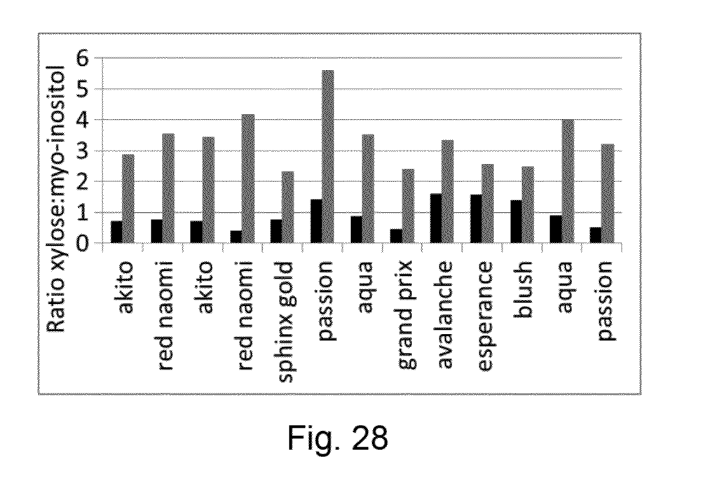

As the level of myo-inositol was virtually independent of the storage duration and temperature, it may serve as a reference sugar to relate a xylose increase to (FIG. 27). A possible relationship between initial myo-inositol and initial xylose would strengthen this approach. Indeed, a weak relationship between initial myo-inositol and xylose was observed (FIG. 28). This means that apart from the absolute amount of xylose, also the ratio between myo-inositol and xylose may be used as a marker for remaining vase life.

In most rose cultivars, the initial level of xylose in freshly harvested flowers is low, and a detection of substantial levels of xylose indicates that the flowers are not fresh and that a reduction of potential shelf life is expected.

The pattern of xylose accumulation in leaves during storage is similar as in petals. However, the absolute levels in leaves are about 5 times lower which may be a disadvantage is the sensitivity of the test method to be used is limiting.

### Changes in Gene Expression

In general there is a steep increase in β-xylosidase gene expression (measured as mRNA abundance compared to pre-storage level) during storage at all temperatures and in both petals and leaves of all rose cultivars tested (Avalanche, Happy Hour, Akito, Red Naomi). In general the change in expression was more pronounced in leaves than in petals and the increase was more pronounced at higher than at lower storage temperature. In petals, especially at higher storage temperatures, relative expression showed a peak, levelling off at longer storage times. In leaves, expression showed a continuous increase over time.

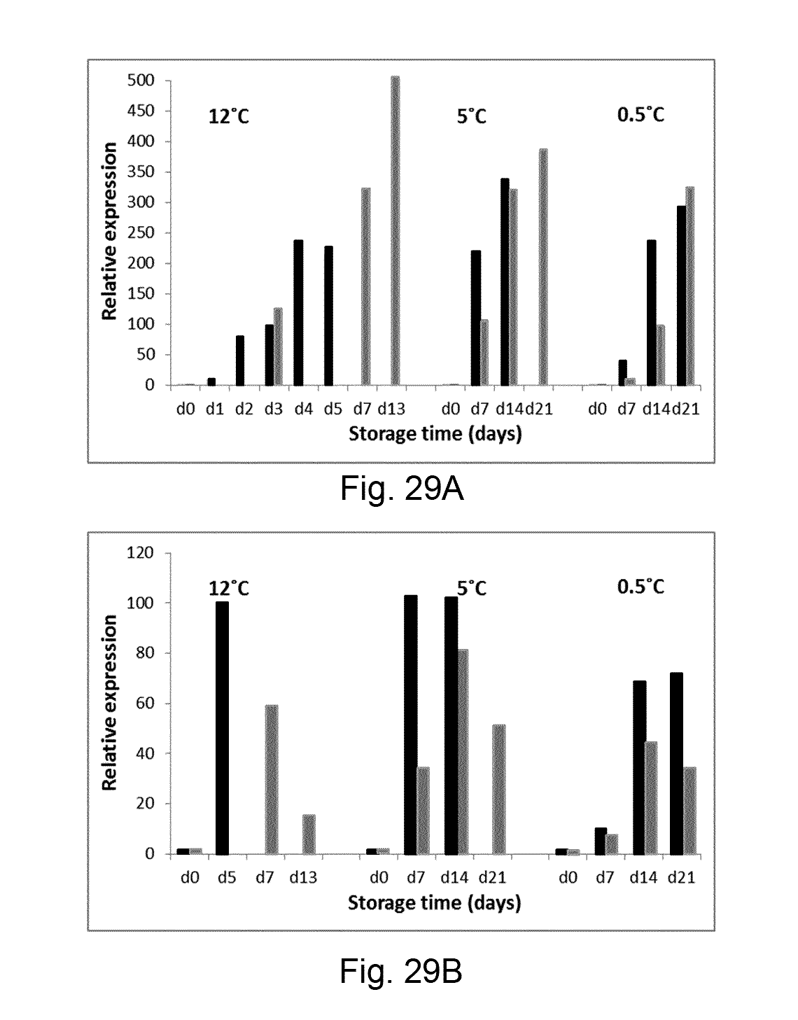

Thus, the trend of increasing beta xylosidase gene expression with storage time is similar for all cultivars tested. However, the maximum relative gene expression level seems cultivar specific. For example, β-xylosidase expression in the leaves can reach mean levels of approximately 250 compared to the initial level in Akito, but reaches much higher levels in the cultivars Avalanche (appr. 800), Happy Hour (appr. 1000) and Red Naomi (appr. 1700). Within one cultivar we have shown that results obtained from independent experiments are quite reproducible, as concluded for example, from the qPCR data performed on the leaves and petals of the rose cultivar Akito (FIG. 29).

### CONCLUSION

The results described above show that the sugar xylose in petals or leaves can be used as a marker for storage history and to predict remaining vase life of roses. The observed changes are more pronounced and at a higher absolute level in petals than in leaves. Development of a rapid and easy test will therefore be easier for petal xylose. The test may make use of the relatively stable sugar alcohol myo-inositol as a reference sugar reflecting the initial situation. Xylose shows an increase over time that is dependent on the storage temperature, i.e. increase is faster at higher storage temperature.

In addition to the metabolite xylose, the expression of β-xylosidase gene (or activity of β-xylosidase enzyme) may be used as a marker. In particular, in leaves there is a continuous increase in expression of this gene with storage time. The expression is more pronounced at higher storage temperatures.

### REFERENCES

Gorin, N.; Berkholst, C E M., 1982: Starch in petals of cut roses, cv Sonia, as a probable criterion of picking. Gartenbauwissenschaft 47(2): 75-77
Berkholst, C E M and Gonzales, M N, 1989. A simple test for starch in rose petals. Advances in Horticultural science 3: 24-28.
Ichimura K., Kishimoto M., Norikoshi R., Kawabata Y. and Yamada K. 2005. Soluble carbohydrates and variation in vase-life of cut rose cultivars ‘Delilah’ and ‘Sonia’. Journal of Horticultural Science & Biotechnology 80(3): 280-286.
Ichimura K., Kohata K., Koketsu M., Yamaguchi Y., Yamaguchi H. and Suto K. 1997. Identification of methyl β-glycopyranoside and xylose as soluble sugar constituents in roses (Rosa hybrida L.). Biosci. Biotech. Biochem. 61(10): 1734-1735.
Ichimura K., Mukasa Y., Fujiwara T., Kohata K., Goto R. and Suto K. 1999b. Possible roles of methyl glucoside and myo-inositol in the opening of cut rose flowers. Annals of Botany 83: 551-557.
Ichimura K., Ueyama S. and Goto R. 1999a. Possible roles of soluble carbohydrates constituents in cut rose flowers. J. Japan. Soc. Hort. Sci. 68(3): 534-539.
Kazuo Ichimura, Katsunori Kohata, Mamoru Koketsu, Misa Shimamura, Akiko Ito, 1998. Identification of pinitol as a main sugar constituent and changes in its content during flower bud development in carnation (Dianthus caryophyllus L.) Journal of Plant Physiology 152: 363-367
Bieleski, Roderick L., Fructan Hydrolysis Drives Petal Expansion in the Ephemeral Daylily Flower. Plant Physiology Vol. 103, No. 1, September, 1993
Kazuo ICHIMURA, *Katsunori KOHATA, *Yuichi YAMAGUCHI, *Mitsuru DOUZONO, *Hiroshi IKEDA, *Mamoru KOKETSU (2000) Identification of L-Inositol and Scyllitol and Their Distribution in Various Organs in Chrysanthemum, Bioscience, Biotechnology, and Biochemistry 64: 865-868
Kazuo Ichimura, Katsunori Kohata, Rie Goto (2000), Soluble carbohydrates in Delphinium and their influence on sepal abscission in cut flowers, Physiologia Plantarum 108: 307-313.

1. A method for determining the vase life or storage history of one or more cut flowers, wherein the method comprises assaying a test sample obtained from the one or more cut flowers for one or more of:
(a) an indicator representative of xylose concentration;
(b) an indicator representative of β-xylosidase expression; and
(c) an indicator representative of β-xylosidase activity;
to determine a value for the one or more indicators in the test sample.

2. A method according to claim 1 wherein an indicator representative of xylose concentration comprises:
(i) xylose concentration; or
(ii) the ratio of xylose concentration to myo-inositol concentration.

3. A method according to claim 1 wherein an indicator representative of β-xylosidase expression comprises:
(i) concentration of β-xylosidase protein or a fragment thereof; or
(ii) expression level of β-xylosidase mRNA, β-xylosidase cDNA or a fragment of either thereof.

4. A method according to any of claim 1 wherein an indicator representative of β-xylosidase activity comprises: (i) β-xylosidase enzyme activity; or (ii) xylose concentration.

5. A method according to claim 1 wherein the cut flowers are of the Rosaceae family.

6. A method according to claim 5 wherein the cut flowers are of the Rosa genus.

7. A method according to claim 1 wherein the test sample comprises or is derived from petal or leaf tissue.

8. A method according to claim 1 further comprising comparing a value for an indicator in the test sample with a value for the indicator in a sample obtained at the time of harvesting the cut flowers.

9. A method according to claim 1 further comprising determining xylose concentration in a test sample comprising or derived from petal tissue.

10. A method according to claim 1 further comprising determining at least one of β-xylosidase expression and β-xylosidase activity in a test sample comprising or derived from leaf tissue.

11. A method according to claim 1 further comprising comparing a value for an indicator in the test sample (test indicator data) with a value for the indicator in a control sample obtained from control cut flowers of known vase life or storage history (control indicator data).

12. A method according to claim 11 wherein the step of comparing the test indicator data with the control indicator data comprises use of a computer implemented model which relates the control indicator data to at least one of a vase life or a storage history of the control cut flowers.

13. A computer-implemented method of obtaining a model for predicting vase life and/or storage history of cut flowers, wherein the method comprises:
(a) receiving a value for one or more of:
(i) an indicator representative of xylose concentration;
(ii) an indicator representative of β-xylosidase expression; and
(iii) an indicator representative of β-xylosidase activity;
in a control sample taken from one or more control cut flowers (control indicator data), wherein the one or more control cut flowers have a known vase life and/or storage history (control vase life or storage history data); and
(b) storing the control indicator data and the control vase life or storage history data in a data storage structure that associates the control indicator data with the control vase life and/or storage history data.

14. A computer implemented method of predicting vase life and/or storage history of cut flowers, the method comprising:
a) receiving a value for one or more of:
(i) an indicator representative of xylose concentration;
(ii) an indicator representative of β-xylosidase expression; and
(iii) an indicator representative of β-xylosidase activity; in a test sample taken from one or more cut flowers (test indicator data); and
b) comparing the test indicator data with control indicator data obtained from one or more control flowers of known vase life and/or storage history, using a data storage structure that associates the control indicator data with the control vase life and/or storage history data, wherein the control indicator data comprises a value for one or more of:
(i) an indicator representative of xylose concentration;
(ii) an indicator representative of β-xylosidase expression; and
(iii) an indicator representative of β-xylosidase activity; in a control sample taken from the one or more control cut flowers.

15. A computer implemented method according to claim 14 wherein step (a) of receiving the test indicator data comprises assaying a test sample obtained from the one or more cut flowers for one or more of:
a) an indicator representative of xylose concentration;
b) an indicator representative of β-xylosidase expression; and
c) an indicator representative of β-xylosidase activity; to determine a value for the one or more indicators in the test sample.

16. A computer program which, when executed on a computer, is arranged to perform a method of claim 13.

17. A computer program according to claim 16 which is stored on a computer-readable medium.


SEQUENCE LISTING (12 sequences)

SEQ ID NO: 1 - DNA, 20 bp, Artificial Sequence
  consensus forward PCR primer for rose beta-xylosidase
caaaggtcccgtggtatttc

SEQ ID NO: 2 - DNA, 19 bp, Artificial Sequence
  consensus reverse PCR primer for rose beta-xylosidase
gtggtggcacttagacttg

SEQ ID NO: 3 - DNA, 22 bp, Artificial Sequence
  forward PCR primer for rose actin
tggagagtgattgggatctttt

SEQ ID NO: 4 - DNA, 22 bp, Artificial Sequence
  reverse PCR primer for rose actin
tccatagcagtttatgaccaca

SEQ ID NO: 5 - DNA, 2708 bp, Arabidopsis thaliana
catgaaaactaaaaaacacaaacatcacatgtatacacacatatagttacaaacacacatacacaaaacacagatatataaaaatgtcttgttataataaagcactattgatcggcaacaaagtcgtcgttatacttgtattcctcttatgtttggttcactcgtcagagtcacttcgaccactgtttgcatgtgatccagcaaacgggttaacccggacgctccggttctgtcgggccaatgtaccgatccatgtgagagttcaagatttgctcggaaggctcacgttgcaggagaagatccgcaacctcgtcaacaatgctgccgccgtaccacgtctcggtattggaggctatgagtggtggtccgaggctctccacggcatttccgacgttggtccaggcgctaagttcggtggtgcttttcccggtgccaccagcttccctcaggtcatcaccaccgcagcttctttcaaccagtctctatgggaagagatcggacgggtggtgtctgatgaggcaagagctatgtacaatggtggcgtggccggtctgacatattggagtccgaatgtgaatatcttgagggacccgcggtggggccgaggccaagaaactcccggagaagatcctatcgttgccgcaaaatatgccgccagctacgtccggggacttcagggtactgctgccggtaaccgccttaaagtcgccgcatgttgcaaacattacactgcttatgatcttgataattggaatggcgtcgaccgtttccacttcaacgctaaggtcacccaacaagatttagaggacacatacaacgtgccattcaaatcatgtgtttacgaaggaaaagtagcgagtgtaatgtgttcgtacaaccaagtcaatggaaagcccacatgtgctgatgaaaatctcttaaagaacactattcgtggtcaatggcgtctcaatgggtacattgtctcagattgtgactctgttgatgttttcttcaaccaacaacattacactagcactccggaagaagccgccgccagatccattaaagccggtttggacttggactgcgggccgtttttggcgattttcacggaaggtgcagtgaagaaaggattgttaacggagaatgacatcaatttagcacttgctaatacattaacagtccaaatgagacttggtatgtttgatggtaaccttgggccgtacgctaatcttgggccaagagatgtttgtactccggcccataaacatttagctcttgaagcagcccatcaagggattgttcttctcaaaaactctgctcgctctcttccactctcccccagacgccaccgcaccgtcgccgtgattggtccaaactccgacgtcactgagactatgatcggcaactatgcagggaaagcatgcgcctatacgtcgccgttgcaagggatttcaagatacgcgaggacacttcaccaagctggctgtgccggcgtggcttgcaaagggaaccaaggatttggtgcagcggaggcagcggcgcgtgaagccgacgcgacggttcttgtgatgggattagatcagtcgatagaggcagagacacgagatcgaaccgggcttctcttaccgggttatcaacaagacctagtgacccgtgtagctcaagcttctagaggtccagtcattttggtccttatgagtggtggaccaatcgatgtaaccttcgctaagaatgatcctcgtgttgctgccatcatttgggctgggtatccgggtcaagcgggtggagctgccatcgccaatatcatctttggtgctgctaatcccggaggaaaactaccaatgacatggtatccacaagattacgtggccaaagtgccaatgacggtaatggccatgagagcatccggtaattatccaggaaggacatacagattctacaaaggtccagtagtatttccatttgggttcggtttaagttacactaccttcactcatagtttggccaaaagcccattggcccaactatcagtttcactctccaatctcaactctgccaataccattctcaactcttcatcacactccatcaaagtgtctcacaccaactgcaattcatttccgaaaatgccccttcacgtcgaagtatcaaacacaggtgaattcgatggaacacacacggtgtttgtatttgctgagccgccgataaacggaataaaaggattgggtgtgaacaaacaattgatagcgttcgagaaggttcatgtcatggcaggggcaaaacagaccgttcaagttgatgttgatgcttgcaagcatcttggtgtagtggatgagtatggaaagaggagaatcccaatgggtgagcataagctgcacattggtgaccttaaacatactattttggtccaaccgcaactttgacggacgcataaaaaaaccaacaaataaggaaagcattttaacaaagtggagtgtttcctcttatttatataatatagagagataggtttattttcttatgaaattattactcaagaaagtatgaatttgtaaagaaccaccaaagggttgcttcttggttgtgtcttttttcctttaatttttttttggaccaaaggtattgtaacttgtggctcccaacataagaaatacaaggagcccttgtaaaatgctgaaactaaagataaatgataaatttgatatatactggatttttagagt

SEQ ID NO: 6 - DNA, 2584 bp, Arabidopsis thaliana
aagtctttcatcacatttccatttttctctcttccataaaacccaaaagaaactaggaagaggagtaaataatatttgttttaaagaaatgattctccacaaaatggcgttcttggccgttattctcttcttcttgataagcagcagcagcgtttgcgttcatagccgtgaaacgtttgcttgcgatacaaaggacgcagcaacagctacactgagattctgccagctttcagttcctataccggagagagtcagagatttgatcggacggttgacattggccgagaaagtgagcttgttagggaacactgcggcggcgataccacgtctaggaatcaaagggtacgagtggtggtcggaggctttacacggcgtttcaaatgtgggacccggtactaagttcggtggggtttaccctgcagccaccagtttccctcaagtcatcaccaccgttgcttctttcaatgcctccttgtgggaatccatcggacgggttgtgtcaaatgaggccagggccatgtacaacggtggagttggtgggcttacgtattggagcccaaacgttaacatattgagggacccacgttggggacgtggacaggaaactcccggtgaagatccagtagtagccggtaaatacgcagcgagctacgtcagagggttacagggaaacgaccgtagccggttaaaagtagctgcttgttgcaaacatttcacagcttacgatctcgataactggaacggcgtcgacagattccatttcaacgctaaggtaagcaagcaagacatagaagacacgttcgacgtaccgttccgtatgtgtgttaaagaaggtaacgttgcgagcattatgtgttcgtacaatcaagttaatggtgttcctacatgtgctgatcctaatctcctcaagaagaccatacgcaatcaatggggtctcaacgggtatatcgtgtctgattgtgactctgtcggtgttttgtacgatacccaacattacactggtactcctgaagaagctgccgctgattccatcaaagctggcttggatttagattgtgggccatttctaggagcccatacaatcgatgcggtgaagaaaaacttgttgcgtgagtccgatgttgataatgccttaatcaacacgctaacagtccaaatgagactaggaatgtttgatggcgatatagcggctcaaccgtacggacaccttggaccggcacacgtgtgtacaccggttcacaaaggactagctctcgaagcagctcaacaaggaatcgtcctactcaaaaatcacggctcgtctctacctctctcaagccaacgtcaccgaactgtcgccgtaattggacctaattcagacgctacggtcacaatgattggtaattatgcaggggttgcttgtggatataccagtccggttcaaggtattaccggttatgctcgaaccattcatcaaaagggttgcgtggacgtacactgcatggatgatagattgttcgatgccgcggttgaagcggctcgtggagctgatgcgacggttcttgtgatgggtttggatcagtctattgaagcggagttcaaggacagaaacagtttgcttttgcctgggaaacaacaagagcttgtctctagagttgctaaggccgctaaaggcccagttatcttagtattgatgtctggtgggcctatcgatatatcttttgctgagaaggatcggaaaattccagcgattgtttgggccgggtatccgggtcaagaaggtggtaccgcaatcgccgatatcttattcggcagtgctaatcccggaggaaagcttccgatgacttggtatccgcaagattatttaaccaatttaccaatgacagaaatgtcgatgcggccggtccattcgaagcggatcccgggtcggacttaccggttctacgacggtccagttgtttacccgttcgggcatggtttgagttacacgcgctttactcacaacatagccgacgcgccaaaagtgattcctatagctgttcgtggaagaaacggcaccgtttcagggaaatcaatccgtgtgacgcacgctaggtgtgatcgtctctctctcggagtccacgtggaagttactaacgttggctcgagagatgggacgcacacaatgcttgtgttctcggctccgccgggtggagaatgggctccgaagaaacagctggttgcttttgagagagtacacgtggcggttggggagaagaagcgtgtgcaggtgaatatacacgtgtgtaagtatttaagtgtagtggaccgagccgggaaccgaaggattccgatcggtgatcatgggattcatattggagatgagagtcatacggtgtcgcttcaagcttctactcttggagtcatcaagtcttgactctgtttttttcttttcacttttcttgttgttcccaaaatatttttaagagattttaatgtttctaacgaaacgaatttgaaaaaggaaatacaaaactagaagaaaatctgtttcttataattcaaaagatgtatttaaaattgaattgtatggcctcggattttttaaaataaaggttgttttcgg

SEQ ID NO: 7 - DNA, 2430 bp, Arabidopsis thaliana
acaaaccacaacaaaaaatctcgagacaaagatacaatggcgagccgaaacagagcactcttctctgtttccactcttttcctctgtttcatcgtctgcatctccgagcaatcgaacaatcagtcttctccagtcttcgcctgtgacgtcaccggaaacccttctcttgccgggcttagattctgcaacgcggggttgagtatcaaagcccgagtcaccgatcttgtcggaagattgacgttggaggagaaaatcgggtttttgaccagcaaagctatcggcgttagccgccttgggattccgtcttacaaatggtggtcggaggcacttcatggcgtctctaacgtcggaggtggtagtcggttcaccggtcaagtccctggcgccactagcttcccacaagttatactcacggccgcttctttcaatgtgtctttgttccaagccattggcaaggttgtatcgacagaggcgagggcaatgtacaatgtgggatcagccggtttaacgttttggtcacctaatgtgaacatattccgggacccgagatggggaagaggacaagagactcccggtgaggacccaacactctcaagcaaatacgcagtggcctacgttaaaggtcttcaggagactgacggtggagatcctaaccgtctcaaagtcgctgcttgctgcaaacactacaccgcctatgatattgacaattggagaaatgtcaatcgtctcactttcaacgctgtggtaaaccaacaagatctggctgatacgtttcaaccaccgttcaagagctgtgtggttgatgggcatgttgctagtgtcatgtgttcttacaaccaagttaacggtaaaccaacatgtgccgatcctgatctgctttccggtgtgatccgcggacaatggcagctcaacggatacattgtttcggattgtgattcggtagatgtgttgttcagaaaacaacactatgctaagactccagaagaagctgtggccaaatctctattggcaggtttggatttgaattgtgatcatttcaatggtcaacacgcgatgggagcggtcaaggcgggtttggtaaacgaaacagctattgacaaagcgatttcaaacaatttcgcgactctgatgcgtttagggttcttcgatggagaccctaagaagcagctctacggtggtcttggtcctaaggatgtttgcaccgctgataaccaagaactcgcaagagatggcgcaagacaaggcattgtcttgcttaagaactctgctggttcgcttccgctctcaccttccgcaatcaaaacattagccgtgatcggaccaaacgccaatgccacagaaacaatgatcggaaactaccacggtgtaccatgcaagtacacaacgccgcttcagggattggcagaaacggtgtcgtctacctatcaactgggatgtaacgtggcttgcgtagatgcggatataggctcagccgtggatctggctgcttctgcggatgctgttgtgctcgtggtgggcgcagaccaatcaattgagagggagggccatgaccgagtcgacctgtatcttcctggaaagcagcaagagcttgtgactcgagttgctatggcagcaagaggaccggtggtgctagttatcatgtccggtggaggatttgacattacattcgccaagaatgataaaaagatcacaagcataatgtgggtcggataccctggtgaagccggtggtctcgccattgctgacgttatcttcggacgtcataatccgagtggaaatttgccgatgacgtggtatcctcaatcgtacgtggaaaaagttccgatgtcaaatatgaacatgagacccgacaaatcaaaggggtatccgggtcggagttacaggttttacaccggagaaaccgtatacgccttcgcagacgcgcttacctacactaaattcgaccatcagctaatcaaagcgccaagactcgtctctctcagtctcgacgagaaccacccttgccgatcatcggagtgccaatctttggacgcgatcggacctcactgcgagaacgccgttgagggaggatcggatttcgaggttcatttgaatgtaaagaacaccggagacagagcgggaagccacacggtgtttctgttcacgacgtcgccgcaagtacacggatctccgattaagcaactactaggatttgagaagattcgtctgggaaagagtgaagaagcggtggttaggtttaacgtcaatgtgtgtaaggatctgagtgtggttgatgagaccgggaagaggaaaatcgcgttaggtcatcatcttctccatgtaggaagcttgaaacactctttgaacattagtgtttgattcgacggctcgttttgtttttaacttaagatattaattatggtaataaaatgagatagcaattttaaaatc

SEQ ID NO: 8 - DNA, 1656 bp, Arabidopsis thaliana
agttatgtggttgatgggaatgtggcgagtgttatgtgttcttacaatcaagttaacggcaaaccgacatgcgctgatccagatctgctctctggtgttatccgcggtgaatggaaattaaatgggtacattgtttcagattgtgattcagtagatgtcttgtataagaaccaacactatacaaagactccagctgaagctgcagccatatctatattggcaggtttggatttaaactgtggttcattcttgggtcaacatacagaggaagcagttaagtcgggtttggtaaacgaggcagctatcgataaagcgatttcgaacaactttttgacccttatgcgtttaggattctttgatggaaacccaaagaaccaaatctatggcgggttaggtcctaccgacgtttgcacgtctgcgaatcaagagctagcagcagatgcagcaagacaaggcattgttctactcaagaatactggatgcttaccgctttctcctaaatcgatcaaaacactagccgtgattggaccaaacgcgaatgtcaccaaaacaatgattggaaactacgaaggcacgccgtgtaaatacacaacaccactacaaggactagccgggacggtatctacaacatatctaccaggctgctccaatgtagcttgtgctgtagcggatgtagccggcgccacgaaactagcagccactgcagatgtgtctgtgcttgtgatcggtgccgatcaatcaatcgaggcagagagccgagacagagtcgacctgcgtcttcctggacagcaacaagagctggtgatccaagtggctaaagcagcaaaaggaccggtcttgctcgtcattatgtccggtggaggtttcgatattacattcgctaagaatgacccaaagatcgccggaattttgtgggttggttatcccggagaagccggtggtatcgccattgctgatatcatctttggccgttataatccaagtgggaaattaccgatgacgtggtatccacagtcgtatgtagagaaagttccgatgacaataatgaacatgagacccgataaagcaagcgggtatccgggtcggacttaccgattctacaccggagaaacagtatacgcattcggagatggactcagctacaccaaattcagtcacactttagtcaaagctccaagtctcgtttctctcggtctcgaagagaatcacgtttgccgatcatcggaatgtcaatcgctagacgcgatcggaccgcactgcgaaaacgctgtctccggcggtggatcggcgtttgaagttcatatcaaggtacgaaacggaggagatagagaagggattcacacggtgtttctattcacgacgccgccggcgattcacggatcgccgaggaagcatttggtaggattcgagaagattcgattggggaagagggaagaagcggtggttaggtttaaggtagagatatgtaaagatctgagtgtggttgatgagattgggaagaggaagattggtttgggaaagcatcttcttcatgtcggagatttaaaacattccttaagcattagaatctgattctatatttttatttgaggaagaaaaaaagaatattaatatgcttaagcttttgcaagttggaaaagaaaagtaataaaaaaa

SEQ ID NO: 9 - protein, 774 aa, Arabidopsis thaliana
MSCYNKALLIGNKVVVILVFLLCLVHSSESLRPLFACDPANGLTRTLRFCRANVPIHVRVQDLLGRLTLQEKIRNLVNNAAAVPRLGIGGYEWWSEALHGISDVGPGAKFGGAFPGATSFPQVITTAASFNQSLWEEIGRVVSDEARAMYNGGVAGLTYWSPNVNILRDPRWGRGQETPGEDPIVAAKYAASYVRGLQGTAAGNRLKVAACCKHYTAYDLDNWNGVDRFHFNAKVTQQDLEDTYNVPFKSCVYEGKVASVMCSYNQVNGKPTCADENLLKNTIRGQWRLNGYIVSDCDSVDVFFNQQHYTSTPEEAAARSIKAGLDLDCGPFLAIFTEGAVKKGLLTENDINLALANTLTVQMRLGMFDGNLGPYANLGPRDVCTPAHKHLALEAAHQGIVLLKNSARSLPLSPRRHRTVAVIGPNSDVTETMIGNYAGKACAYTSPLQGISRYARTLHQAGCAGVACKGNQGFGAAEAAAREADATVLVMGLDQSIEAETRDRTGLLLPGYQQDLVTRVAQASRGPVILVLMSGGPIDVTFAKNDPRVAAIIWAGYPGQAGGAAIANIIFGAANPGGKLPMTWYPQDYVAKVPMTVMAMRASGNYPGRTYRFYKGPVVFPFGFGLSYTTFTHSLAKSPLAQLSVSLSNLNSANTILNSSSHSIKVSHTNCNSFPKMPLHVEVSNTGEFDGTHTVFVFAEPPINGIKGLGVNKQLIAFEKVHVMAGAKQTVQVDVDACKHLGVVDEYGKRRIPMGEHKLHIGDLKHTILVQPQL

SEQ ID NO: 10 - protein, 768 aa, Arabidopsis thaliana
MILHKMAFLAVILFFLISSSSVCVHSRETFACDTKDAATATLRFCQLSVPIPERVRDLIGRLTLAEKVSLLGNTAAAIPRLGIKGYEWWSEALHGVSNVGPGTKFGGVYPAATSFPQVITTVASFNASLWESIGRVVSNEARAMYNGGVGGLTYWSPNVNILRDPRWGRGQETPGEDPVVAGKYAASYVRGLQGNDRSRLKVAACCKHFTAYDLDNWNGVDRFHFNAKVSKQDIEDTFDVPFRMCVKEGNVASIMCSYNQVNGVPTCADPNLLKKTIRNQWGLNGYIVSDCDSVGVLYDTQHYTGTPEEAAADSIKAGLDLDCGPFLGAHTIDAVKKNLLRESDVDNALINTLTVQMRLGMFDGDIAAQPYGHLGPAHVCTPVHKGLALEAAQQGIVLLKNHGSSLPLSSQRHRTVAVIGPNSDATVTMIGNYAGVACGYTSPVQGITGYARTIHQKGCVDVHCMDDRLFDAAVEAARGADATVLVMGLDQSIEAEFKDRNSLLLPGKQQELVSRVAKAAKGPVILVLMSGGPIDISFAEKDRKIPAIVWAGYPGQEGGTAIADILFGSANPGGKLPMTWYPQDYLTNLPMTEMSMRPVHSKRIPGRTYRFYDGPVVYPFGHGLSYTRFTHNIADAPKVIPIAVRGRNGTVSGKSIRVTHARCDRLSLGVHVEVTNVGSRDGTHTMLVFSAPPGGEWAPKKQLVAFERVHVAVGEKKRVQVNIHVCKYLSVVDRAGNRRIPIGDHGIHIGDESHTVSLQASTLGVIKS

SEQ ID NO: 11 - protein, 773 aa, Arabidopsis thaliana
MASRNRALFSVSTLFLCFIVCISEQSNNQSSPVFACDVTGNPSLAGLRFCNAGLSIKARVTDLVGRLTLEEKIGFLTSKAIGVSRLGIPSYKWWSEALHGVSNVGGGSRFTGQVPGATSFPQVILTAASFNVSLFQAIGKVVSTEARAMYNVGSAGLTFWSPNVNIFRDPRWGRGQETPGEDPTLSSKYAVAYVKGLQETDGGDPNRLKVAACCKHYTAYDIDNWRNVNRLTFNAVVNQQDLADTFQPPFKSCVVDGHVASVMCSYNQVNGKPTCADPDLLSGVIRGQWQLNGYIVSDCDSVDVLFRKQHYAKTPEEAVAKSLLAGLDLNCDHFNGQHAMGAVKAGLVNETAIDKAISNNFATLMRLGFFDGDPKKQLYGGLGPKDVCTADNQELARDGARQGIVLLKNSAGSLPLSPSAIKTLAVIGPNANATETMIGNYHGVPCKYTTPLQGLAETVSSTYQLGCNVACVDADIGSAVDLAASADAVVLVVGADQSIEREGHDRVDLYLPGKQQELVTRVAMAARGPVVLVIMSGGGFDITFAKNDKKITSIMWVGYPGEAGGLAIADVIFGRHNPSGNLPMTWYPQSYVEKVPMSNMNMRPDKSKGYPGRSYRFYTGETVYAFADALTYTKFDHQLIKAPRLVSLSLDENHPCRSSECQSLDAIGPHCENAVEGGSDFEVHLNVKNTGDRAGSHTVFLFTTSPQVHGSPIKQLLGFEKIRLGKSEEAVVRFNVNVCKDLSVVDETGKRKIALGHHLLHVGSLKHSLNISV

SEQ ID NO: 12 - protein, 784 aa, Arabidopsis thaliana
MGSSSPLTRRNRAPPSSVSSVYLIFLCFFLYFLNFSNAQSSPVFACDVAANPSLAAYGFCNTVLKIEYRVADLVARLTLQEKIGFLVSKANGVTRLGIPTYEWWSEALHGVSYIGPGTHFSSQVPGATSFPQVILTAASFNVSLFQAIGKVVSTEARAMYNVGLAGLTYWSPNVNIFRDPRWGRGQETPGEDPLLASKYASGYVKGLQETDGGDSNRLKVAACCKHYTAYDVDNWKGVERYSFNAVVTQQDMDDTYQPPFKSCVVDGNVASVMCSYNQVNGKPTCADPDLLSGVIRGEWKLNGYIVSDCDSVDVLYKNQHYTKTPAEAAAISILAGLDLNCGSFLGQHTEEAVKSGLVNEAAIDKAISNNFLTLMRLGFFDGNPKNQIYGGLGPTDVCTSANQELAADAARQGIVLLKNTGCLPLSPKSIKTLAVIGPNANVTKTMIGNYEGTPCKYTTPLQGLAGTVSTTYLPGCSNVACAVADVAGATKLAATADVSVLVIGADQSIEAESRDRVDLHLPGQQQELVIQVAKAAKGPVLLVIMSGGGFDITFAKNDPKIAGILWVGYPGEAGGIAIADIIFGRYNPSGKLPMTWYPQSYVEKVPMTIMNMRPDKASGYPGRTYRFYTGETVYAFGDGLSYTKFSHTLVKAPSLVSLGLEENHVCRSSECQSLDAIGPHCENAVSGGGSAFEVHIKVRNGGDREGIHTVFLFTTPPAIHGSPRKHLVGFEKIRLGKREEAVVRFKVEICKDLSVVDEIGKRKIGLGKHLLHVGDLKHSLSIRI